(19) United States
(12) Patent Application Publication
(10) Pub. No.: US 2021/0038829 A1
(43) Pub. Date: Feb. 11, 2021

(54) MEDICAMENT DEVICE FOR USE WITH A CONVENTIONAL SYRINGE

(71) Applicants: Steven M. Montgomery, Los Angeles, CA (US); Gregory E. Mote, Big Bear Lake, CA (US)

(72) Inventors: Steven M. Montgomery, Los Angeles, CA (US); Gregory E. Mote, Big Bear Lake, CA (US)

(21) Appl. No.: 17/083,129

(22) Filed: Oct. 28, 2020

Related U.S. Application Data
(63) Continuation-in-part of application No. 16/241,884, filed on Jan. 7, 2019.
Application No. 16/241,884 is a continuation-in-part of application No. 15/981,721, filed on May 16, 2018.
Application No. 15/981,721 is a continuation-in-part of application No. 15/707,111, filed on Sep. 18, 2017, now Pat. No. 10,463,804.
(60) Provisional application No. 62/507,077, filed on May 16, 2017.

(51) Int. Cl. A61M 5/32; A61M 5/20; A61M 5/315

(52) U.S. Cl. CPC A61M 5/3204; A61M 5/31501; A61M 5/2033

(57) ABSTRACT

A novel medicament device is disclosed having a body assembly for receiving a conventional syringe. The body assembly preferably includes an upper housing, an inner housing adapted to matingly engage the upper housing, a lower housing receiving the inner housing, and a sheath slideably contained within the lower housing. The device preferably further includes a sheath lock mechanism and a plunger lock mechanism. A sheath cap, which may include a needle cover stripper and stripper cap, may be disposed about the lower housing. With any of the disclosed embodiments, both trained and untrained users may administer an injection safely and conveniently to themselves or others. As a common syringe may be utilized, engineering and production issues of conventional automatic injectors are avoided. Moreover, with the disclosed devices, the syringe needle is not directly exposed and automatically locks out post injection, avoiding accidental needle sticks and unsuitable syringe disposal.

### CROSS-REFERENCE TO RELATED APPLICATIONS

The present application claims benefit of and priority to U.S. application Ser. No. 16/241,884 filed on Jan. 7, 2019; U.S. application Ser. No. 15/981,721 filed on May 16, 2018; U.S. application Ser. No. 15/707,111 filed on Sep. 18, 2017, now U.S. Pat. No. 10,463,804; U.S. Application No. 62/507,077 filed on May 16, 2017; and U.S. Application No. 62/398,515 filed on Sep. 22, 2016. Each application is incorporated by reference herein.

### BACKGROUND

The present disclosure relates generally to a device for injecting or otherwise administrating a medication.

### SUMMARY

One exemplary embodiment of the disclosed subject matter is a device for administering medicament using a conventional syringe. The device has a body assembly for receiving the syringe. The body assembly includes an upper housing, an inner housing adapted to matingly engage the upper housing, a lower housing receiving the inner housing, and a sheath slideably contained within the lower housing. An inner cap is disposed about the upper housing. The device may include a sheath lock mechanism including a sheath lock disposed within the lower housing, guide rods disposed about the sheath, and springs encircling the guide rods. The sheath lock has sheath lock legs adapted to engage the sheath to prohibit movement of the sheath after use of the device.

The medicament device may further include a plunger lock mechanism for maintaining the syringe inside of the body assembly. The plunger lock mechanism has bayonet lock arms and a bayonet lock channel. The bayonet lock arms extend from the upper housing. The bayonet lock channel is cut into the inner housing and is adapted to receive the bayonet lock arms. During operation of the medicament device, the sheath is adapted to engage the inner housing once the syringe has been fully inserted into an injection site. Doing so disengages the upper housing and inner cap to permit medication inside the syringe to be dispensed.

The medicament device may include a sheath cap disposed about the lower housing. The sheath cap has a needle cover stripper and a stripper cap for receiving the needle cover stripper. The needle cover stripper includes ramps on the interior surface thereof, wherein the stripper cap includes cap fingers adapted to engage the ramps.

The inner housing of the medicament device may include guide rod tunnels, each of which respectively receives one end of a guide rod. An optional outer cap is adapted to receive the inner cap. An optional wipe pad may be disposed about the stripper cap. A foil seal covers the wipe pad and may be peeled off to permit use of the wipe pad for cleaning of the injection site pre and post injection.

Another exemplary embodiment of the disclosed subject matter is a device for administering medicament using a conventional syringe, wherein the device includes a housing, a sheath, and a sheath lock mechanism. The housing has a track cut into it. The sheath has a body and legs extending upward therefrom, wherein the sheath is configured to slide within the housing. The sheath lock mechanism includes a sheath lock and a spring. The sheath lock is configured to slide within the track. The spring is disposed between the sheath and the housing. The sheath lock has a body, first and second legs extending downward therefrom, and a third leg extending upward from the body. One of the legs of the sheath has a ledge, wherein the ledge is adapted to engage the sheath lock to prohibit movement of the sheath once medicament within a syringe has been fully administered by a user.

The first and second legs of the sheath lock are substantially parallel to one another, wherein the second leg of the sheath lock includes a sheath stop. The sheath stop is disposed near the ledge of the sheath during engagement of the sheath lock mechanism to prohibit movement of the sheath after use of the device. The device may also include a top cap configured to slide over the housing. The housing may include a stop hole for receiving a sprung tab extending from the top cap when the top cap has been slid over the housing. Each leg of the sheath includes a ramp configured to deflect the sprung tab away from the housing to cause the sprung tab to disengage from the stop hole during use of the device.

The device may include a plunger lock mechanism having a lock slot cut within the top cap, and a plunger lock adapted to fit within the lock slot. The housing may include lower and upper holes therewithin. The first leg of the sheath lock has a catch. The catch is adapted to be received in the lower hole during an initial stage of use of the device. The catch is also adapted to be received in the upper hole during a later stage of use of the device by way of the sheath lock sliding upward within the track of the housing.

The medicament device may include a sheath cap disposed about the sheath before use of the device. The sheath cap includes finger pads cut therewithin, and grippers in its interior. The grippers are adapted to engage a cover of a syringe to remove the cover.

Yet another exemplary embodiment of the disclosed subject matter is a medicament device having a housing with a track cut into a side and running between the top end and bottom end thereof, a syringe catch, retaining slots cut within the syringe catch, and a channel cut into an interior of the housing. A sheath is configured to slide within the housing. The sheath has a body and a traveler extending outward therefrom. The track is adapted to receive the traveler. After medication has been injection by a user with a conventional syringe, the channel is adapted to receive the traveler by rotating the body of the sheath downward away from the housing. A spring is disposed between the sheath and the housing.

The medicament device may also include a top cap configured to slide over the housing, wherein the housing includes a body and arms extending away from the body at one end thereof. The arms of the top cap are adapted to engage the syringe catch.

A sheath cap may be disposed about the sheath before use of the device, wherein the sheath cap includes finger pads cut therewithin and grippers in the interior thereof. The grippers are adapted to engage a cover of a syringe.

### BRIEF DESCRIPTION OF THE DRAWINGS

Some non-limiting exemplary embodiments of the disclosed subject matter are illustrated in the following drawings. Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described. Dimensions of components and features shown in the figures are chosen for convenience or clarity of presentation. For convenience or clarity, some elements or structures are not shown or shown only partially and/or with different perspective or from different point of views.

FIG. 1 illustrates an embodiment of a medicament device disclosed herein in use;

FIG. 2 is a partially exploded view of the medicament device seen in FIG. 1 with sheath cap attached at the bottom end of the device;

FIG. 3A is a perspective view of the medicament device seen in FIG. 2;

FIG. 3B is a fully exploded view of the medicament device seen in FIG. 2;

FIG. 4A is a front view of the medicament device seen in FIG. 2;

FIG. 4B is a sectional view of the medicament device taken along line 4B-4B seen in FIG. 4A;

FIG. 5A is a front view of the medicament device seen in FIG. 2 with the sheath cap removed;

FIG. 5B is a sectional view of the medicament device taken along line 5B-5B seen in

FIG. 5A;

FIG. 6A is a front view of the medicament device seen in FIG. 1 disposed above the site to be injected;

FIG. 6B is a sectional view of the medicament device and site taken along line 6B-6B seen in FIG. 6A;

FIG. 7A is a front view of the medicament device seen in FIG. 1, wherein the sheath is touching the skin of the site to be injected;

FIG. 7B is a sectional view of the medicament device and site taken along line 7B-7B seen in FIG. 7A;

FIG. 8A is a front view of the medicament device seen in FIG. 1, wherein the needle of the syringe has been inserted into the skin;

FIG. 8B is a sectional view of the medicament device and site taken along line 8B-8B seen in FIG. 8A;

FIG. 9A is a front view of the medicament device seen in FIG. 1, wherein medicament contained in the syringe is injected into the site;

FIG. 9B is a sectional view of the medicament device and site taken along line 9B-9B seen in FIG. 9A;

FIG. 10A is a front view of the medicament device seen in FIG. 1, wherein the device is in a partially released state after the medicament contained in the syringe has been injected into the site;

FIG. 10B is a sectional view of the medicament device and site taken along line 10B-10B seen in FIG. 10A;

FIG. 11A is a front view of the medicament device seen in FIG. 1, wherein the device is in a fully released state after the medicament contained in the syringe has been injected into the site;

FIG. 11B is a sectional view of the medicament device and site taken along line 11B-11B seen in FIG. 11A;

FIG. 12A is a front view of the medicament device seen in FIG. 1 disposed directly above the site after injection;

FIG. 12B is a sectional view of the medicament device and site taken along line 12B-12B seen in FIG. 12A;

FIG. 13 illustrates a partially exploded view of another embodiment of a medicament device disclosed herein;

FIG. 14A is a perspective view of the medicament device seen in FIG. 13;

FIG. 14B is a fully exploded view of the medicament device seen in FIG. 13;

FIG. 15A is a front view of the medicament device seen in FIG. 13;

FIG. 15B is a side view of the medicament device seen in FIG. 13;

FIG. 15C is a sectional view of the medicament device taken along line 15C-15C seen in FIG. 15A;

FIG. 15D is a sectional view of the medicament device taken along line 15D-15D seen in FIG. 15B;

FIG. 16A is a front view of the medicament device seen in FIG. 13 with the sheath cap removed;

FIG. 16B is a side view of the medicament device seen in FIG. 13 with the sheath cap removed;

FIG. 16C is a sectional view of the medicament device taken along line 16C-16C seen in FIG. 16A;

FIG. 16D is a sectional view of the medicament device taken along line 16D-16D seen in FIG. 16B;

FIG. 17A is a front view of the medicament device seen in FIG. 16A with the needle deployed;

FIG. 17B is a side view of the medicament device seen in FIG. 16A with the needle deployed;

FIG. 17C is a sectional view of the medicament device taken along line 17C-17C seen in FIG. 17A;

FIG. 17D is a sectional view of the medicament device taken along line 17D-17D seen in FIG. 17B;

FIG. 18A is a front view of the medicament device seen in FIG. 16A with medicament in the process of being injected;

FIG. 18B is a side view of the medicament device seen in FIG. 16A with medicament in the process of being injected;

FIG. 18C is a sectional view of the medicament device taken along line 18C-18C seen in FIG. 18A;

FIG. 18D is a sectional view of the medicament device taken along line 18D-18D seen in FIG. 18B;

FIG. 19A is a front view of the medicament device seen in FIG. 16A, wherein the device is in a fully released state after the medicament contained in the syringe has been injected into the site;

FIG. 19B is a side view of the medicament device seen in FIG. 16A, wherein the device is in a fully released state after the medicament contained in the syringe has been injected into the site;

FIG. 19C is a sectional view of the medicament device taken along line 19C-19C seen in FIG. 19A;

FIG. 19D is a sectional view of the medicament device taken along line 19D-19D seen in FIG. 19B;

FIG. 20A is a front view of the medicament device seen in FIG. 16A, wherein the device is in a fully released state after the medicament contained in the syringe has been injected into the site and after the device has been recapped for disposal;

FIG. 20B is a side view of the medicament device seen in FIG. 16A, wherein the device is in a fully released state after the medicament contained in the syringe has been injected into the site and after the device has been recapped for disposal;

FIG. 20C is a sectional view of the medicament device taken along line 20C-20C seen in FIG. 20A;

FIG. 20D is a sectional view of the medicament device taken along line 20D-20D seen in FIG. 20B;

FIG. 21A is a perspective view of the sleeve of the medicament device of FIG. 13;

FIG. 21B is a perspective view of the sheath of the medicament device of FIG. 13;

FIG. 21C is a front view of the sheath seen in FIG. 21B;

FIG. 21D is a sectional view of the sheath taken along line 21D-21D seen in FIG. 21C;

FIG. 22 illustrates a partially exploded view of yet another embodiment of a medicament device disclosed herein;

FIG. 23A is a perspective view of the medicament device seen in FIG. 22;

FIG. 23B is a fully exploded view of the medicament device seen in FIG. 22;

FIG. 24A is a front view of the medicament device seen in FIG. 22;

FIG. 24B is a side view of the medicament device seen in FIG. 22;

FIG. 24C is a sectional view of the medicament device taken along line 24C-24C seen in FIG. 24B;

FIG. 25A is a front view of the medicament device seen in FIG. 22 with the sheath cap removed;

FIG. 25B is a side view of the medicament device seen in FIG. 22 with the sheath cap removed;

FIG. 25C is a sectional view of the medicament device taken along line 25C-25C seen in FIG. 25B;

FIG. 26A is a front view of the medicament device seen in FIG. 25A with the needle deployed;

FIG. 26B is a side view of the medicament device seen in FIG. 25A with the needle deployed;

FIG. 26C is a sectional view of the medicament device taken along line 26C-26C seen in FIG. 26B;

FIG. 27A is a front view of the medicament device seen in FIG. 25A with medicament in the process of being injected;

FIG. 27B is a side view of the medicament device seen in FIG. 27A with medicament in the process of being injected;

FIG. 27C is a sectional view of the medicament device taken along line 27C-27C seen in FIG. 27B;

FIG. 28A is a front view of the medicament device seen in FIG. 25A, wherein the device is in a fully released state after the medicament contained in the syringe has been injected into the site;

FIG. 28B is a side view of the medicament device seen in FIG. 25A, wherein the device is in a fully released state after the medicament contained in the syringe has been injected into the site;

FIG. 28C is a sectional view of the medicament device taken along line 28C-28C seen in FIG. 28B;

FIG. 29A is a front view of the medicament device seen in FIG. 25A, wherein the device is in a fully released state after the medicament contained in the syringe has been injected into the site and after the device has been recapped for disposal;

FIG. 29B is a side view of the medicament device seen in FIG. 25A, wherein the device is in a fully released state after the medicament contained in the syringe has been injected into the site and after the device has been recapped for disposal;

FIG. 29C is a sectional view of the medicament device taken along line 29C-29C seen in FIG. 29B;

FIG. 30A is a perspective view of the sleeve of the medicament device of FIG. 22;

FIG. 30B is a perspective view of the sheath of the medicament device of FIG. 22;

FIG. 30C is a top view of the sheath seen in FIG. 30B;

FIG. 30D is a sectional view of the medicament device taken along line 30D-30D seen in FIG. 30C;

FIG. 30E is a perspective view of the pusher of the medicament device of FIG. 22;

FIG. 31 illustrates a partially exploded view of yet another embodiment of a medicament device disclosed herein;

FIG. 32A is a perspective view of the medicament device seen in FIG. 31;

FIG. 32B is a fully exploded view of the medicament device seen in FIG. 31;

FIG. 33A is a front view of the medicament device seen in FIG. 31;

FIG. 33B is a side view of the medicament device seen in FIG. 31;

FIG. 33C is a sectional view of the medicament device taken along line 33C-33C seen in FIG. 33B;

FIG. 34A is a front view of the medicament device seen in FIG. 32 with the sheath cap removed;

FIG. 34B is a side view of the medicament device seen in FIG. 32 with the sheath cap removed;

FIG. 34C is a sectional view of the medicament device taken along line 34C-34C seen in FIG. 34B;

FIG. 35A is a front view of the medicament device seen in FIG. 33A with the needle deployed;

FIG. 35B is a side view of the medicament device seen in FIG. 33A with the needle deployed;

FIG. 35C is a sectional view of the medicament device taken along line 35C-35C seen in FIG. 35B;

FIG. 36A is a front view of the medicament device seen in FIG. 34A with medicament in the process of being injected;

FIG. 36B is a side view of the medicament device seen in FIG. 34A with medicament in the process of being injected;

FIG. 36C is a sectional view of the medicament device taken along line 36C-38C seen in FIG. 36B;

FIG. 37A is a front view of the medicament device seen in FIG. 36A, wherein medicament contained in the syringe has been injected into the site;

FIG. 37B is a side view of the medicament device seen in FIG. 36A, wherein medicament contained in the syringe has been injected into the site;

FIG. 37C is a sectional view of the medicament device taken along line 37C-37C seen in FIG. 37B;

FIG. 38A is a front view of the medicament device seen in FIG. 37A, wherein the device is in a fully released state after the medicament contained in the syringe has been injected into the site;

FIG. 38B is a side view of the medicament device seen in FIG. 37A, wherein the device is in a fully released state after the medicament contained in the syringe has been injected into the site;

FIG. 38C is a sectional view of the medicament device taken along line 38C-38C seen in FIG. 38B;

FIG. 39A is a front view of the medicament device seen in FIG. 38A, wherein the device is in a fully released state after the medicament contained in the syringe has been injected into the site and after the device has been recapped for disposal;

FIG. 39B is a side view of the medicament device seen in FIG. 38A, wherein the device is in a fully released state after the medicament contained in the syringe has been injected into the site and after the device has been recapped for disposal;

FIG. 39C is a sectional view of the medicament device taken along line 39C-39C seen in FIG. 39B;

FIG. 40A is a perspective view of the sleeve of the medicament device of FIG. 31;

FIG. 40B is a perspective view of the sheath of the medicament device of FIG. 31;

FIG. 40C is a side view of the sheath of the medicament device of FIG. 31;

FIG. 40D is a sectional view of the top taken along line 40D-40D seen in FIG. 40C;

FIG. 41A is a perspective view of the pusher of the medicament device of FIG. 31;

FIG. 41B is a side view of the top of the device of FIG. 31;

FIG. 41C is a perspective view of the top taken along line 41C-41C seen in FIG. 41B;

FIG. 42 illustrates a partially exploded view of yet another embodiment of a medicament device disclosed herein;

FIG. 43A is a perspective view of the sheath cap of the medicament device seen in FIG. 42;

FIG. 43B is a top view of the sheath cap seen in FIG. 43A;

FIG. 43C is a front view of the sheath cap seen in FIG. 43A;

FIG. 43D is a bottom view of the sheath cap seen in FIG. 43A;

FIG. 43E is a side view of the sheath cap seen in FIG. 43A;

FIG. 43F is a sectional view of the sheath cap taken along line 43F-43F seen in FIG. 43E;

FIG. 44A is a sectional view of the medicament device seen in FIG. 42 with the housing cap merely covering a portion of the syringe plunger of the syringe having its needle cover in place;

FIG. 44B is a sectional view of the medicament device seen in FIG. 42 with the housing cap in place against the body assembly wherein the syringe cover is seated within the cover stripper;

FIG. 44C is a sectional view of the medicament device seen in FIG. 42 with the sheath cap removed and holding the syringe cover after it has been seated as seen in FIG. 44B;

FIG. 45A illustrates another embodiment of a medicament device disclosed herein;

FIG. 45B illustrates a partially exploded view of the medicament device seen in FIG. 45A;

FIG. 46 illustrates a perspective view of the housing cap of the medicament device seen in FIGS. 45A-45B, wherein the housing cap is inverted from the position seen in FIGS. 45A-45B and the view is looking at the underside of the housing cap;

FIG. 46B illustrates a sectional view of the housing cap of the medicament device seen in FIG. 46A;

FIG. 47 illustrates a perspective view of the sheath with its sheath arms extending from the body of the sheath;

FIGS. 48A-48D respectively illustrate a sectional view of the medicament device seen in FIGS. 45A-45B in different stages of operation;

FIG. 49 illustrates a perspective view of another embodiment of a medicament device disclosed herein;

FIG. 50 is a fully exploded view of the medicament device seen in FIG. 49 with optional outer cap, wherein the device may use a conventional syringe;

FIG. 51A is a front, partially exploded view of the medicament device seen in FIG. 50 showing a conventional syringe with its cover disposed about its needle and medication in its barrel;

FIG. 51B is a partial sectional view of the medicament device seen in FIG. 51A;

FIG. 51C is a partial sectional side view of the medicament device seen in FIG. 51A;

FIG. 52A is a front view of the medicament device seen in FIG. 51A without the upper housing, inner cap, and outer cap, wherein the syringe is disposed about the inner housing;

FIG. 52B is a sectional view of the medicament device seen in FIG. 52A;

FIG. 52C is a sectional side view of the medicament device seen in FIG. 52A;

FIG. 53A is a front view of the medicament device seen in FIG. 50 wherein the syringe is now locked inside the device via the plunger lock mechanism;

FIG. 53B is a sectional view of the medicament device seen in FIG. 53A;

FIG. 53C is a sectional side view of the medicament device seen in FIG. 53A;

FIG. 54A is a front view of the medicament device seen in FIG. 53A with outer cap;

FIG. 54B is a sectional view of the medicament device seen in FIG. 54A;

FIG. 54C is a sectional side view of the medicament device seen in FIG. 54A;

FIG. 55A is a front view of the medicament device seen in FIG. 54A wherein the cover of the syringe has now been stripped via sheath cap and retained within needle cover stripper, which is in turn retained within stripper cap;

FIG. 55B is a sectional view of the medicament device seen in FIG. 55A;

FIG. 55C is a sectional side view of the medicament device seen in FIG. 55A;

FIG. 56A is a front view of the medicament device seen in FIG. 55A without the sheath cap wherein the device is in in the process of its administering an injection by a user against an injection site (not shown), illustrating how the sheath is pushed upward within lower housing to leave the syringe needle exposed;

FIG. 56B is a sectional view of the medicament device seen in FIG. 56A;

FIG. 56C is a sectional side view of the medicament device seen in FIG. 56A;

FIG. 57A is a front view of the medicament device seen in FIG. 56A wherein the plunger has been released and the medication in the barrel of the syringe injected into the injection site;

FIG. 57B is a sectional view of the medicament device seen in FIG. 57A;

FIG. 57C is a sectional side view of the medicament device seen in FIG. 57A;

FIG. 58A is a front view of the medicament device seen in FIG. 57A wherein the medication has been fully injected and the sheath lock mechanism engaged to lock the sheath safely in place around the syringe needle;

FIG. 58B is a sectional view of the medicament device seen in FIG. 58A;

FIG. 58C is a sectional side view of the medicament device seen in FIG. 58A;

FIG. 59A is a front view of the medicament device seen in FIG. 58A with the sheath cap containing the syringe cover has been placed back onto the device;

FIG. 59B is a sectional view of the medicament device seen in FIG. 59A;

FIG. 59C is a sectional side view of the medicament device seen in FIG. 59A;

FIG. 60 illustrates a perspective view of another embodiment of a medicament device disclosed herein;

FIG. 61 is a fully exploded view of the medicament device seen in FIG. 60, wherein the device may use a conventional syringe;

FIG. 62A is a front view of the medicament device seen in FIG. 60 with the syringe locked in place;

FIG. 62B is a sectional view of the medicament device seen in FIG. 62A;

FIG. 62C is a sectional side view of the medicament device seen in FIG. 62A;

FIG. 63A is a front, partially exploded view of the medicament device seen in FIG. 60 but without its top cap, showing how a conventional syringe with its cover disposed about its needle and medication in its barrel is to be dropped into the housing of the device;

FIG. 63B is a partial sectional view of the medicament device seen in FIG. 63A;

FIG. 63C is a partial sectional side view of the medicament device seen in FIG. 63A;

FIG. 64A is a front view of the medicament device seen in FIG. 63A wherein the syringe has now been dropped into the housing;

FIG. 64B is a sectional view of the medicament device seen in FIG. 64A;

FIG. 64C is a sectional side view of the medicament device seen in FIG. 64A;

FIG. 65A is a front view of the medicament device seen in FIG. 64A wherein the syringe is now locked inside the housing via a plunger lock mechanism;

FIG. 65B is a sectional view of the medicament device seen in FIG. 65A;

FIG. 65C is a sectional side view of the medicament device seen in FIG. 65A;

FIG. 66A is a front view of the medicament device seen in FIG. 65A with top cap added and plunger lock in its open position;

FIG. 66B is a sectional view of the medicament device seen in FIG. 66A;

FIG. 66C is a sectional side view of the medicament device seen in FIG. 66A;

FIG. 67A is a front view of the medicament device seen in FIG. 66A wherein the plunger lock is now in its closed position to secure the plunger in place;

FIG. 67B is a sectional view of the medicament device seen in FIG. 67A;

FIG. 67C is a sectional side view of the medicament device seen in FIG. 67A;

FIG. 68A is a front view of the medicament device seen in FIG. 67A wherein the sheath cap has been removed with the syringe cover contained within the sheath cap;

FIG. 68B is a sectional view of the medicament device seen in FIG. 68A;

FIG. 68C is a sectional side view of the medicament device seen in FIG. 68A;

FIG. 69A is a front view of the medicament device seen in FIG. 68A wherein the device is in in the process of its administering an injection by a user against an injection site (not shown), illustrating how the sheath is pushed upward within the housing to leave the syringe needle exposed;

FIG. 69B is a sectional view of the medicament device seen in FIG. 69A;

FIG. 69C is a sectional side view of the medicament device seen in FIG. 69A;

FIG. 70A is a front view of the medicament device seen in FIG. 69A wherein the plunger has been released and the medication in the barrel of the syringe injected into the injection site;

FIG. 70B is a sectional view of the medicament device seen in FIG. 70A;

FIG. 70C is a sectional side view of the medicament device seen in FIG. 70A;

FIG. 71A is a front view of the medicament device seen in FIG. 70A wherein the medication has been fully injected and the sheath lock mechanism engaged to lock the sheath safely in place around the syringe needle;

FIG. 71B is a sectional view of the medicament device seen in FIG. 71A;

FIG. 71C is a sectional side view of the medicament device seen in FIG. 71A;

FIG. 72 illustrates a perspective view of another embodiment of a medicament device disclosed herein;

FIG. 73 is a fully exploded view of the medicament device seen in FIG. 72, wherein the device may use a conventional syringe;

FIG. 74A is a front view of the medicament device seen in FIG. 72 with the syringe locked in place;

FIG. 74B is a sectional view of the medicament device seen in FIG. 74A;

FIG. 74C is a sectional side view of the medicament device seen in FIG. 74A;

FIG. 75A is a front, partially exploded view of the medicament device seen in FIG. 72 but without its top cap, showing how a conventional syringe with its cover disposed about its needle and medication in its barrel is to be dropped into the housing of the device;

FIG. 75B is a partial sectional view of the medicament device seen in FIG. 75A;

FIG. 75C is a partial sectional side view of the medicament device seen in FIG. 75A;

FIG. 76A is a front view of the medicament device seen in FIG. 75A wherein the syringe has now been dropped into the housing;

FIG. 76B is a sectional view of the medicament device seen in FIG. 76A;

FIG. 76C is a sectional side view of the medicament device seen in FIG. 76A;

FIG. 77A is a front view of the medicament device seen in FIG. 64A wherein the syringe is now locked inside the housing;

FIG. 77B is a sectional view of the medicament device seen in FIG. 77A;

FIG. 77C is a sectional side view of the medicament device seen in FIG. 77A;

FIG. 78A is a front view of the medicament device seen in FIG. 65A with top cap added;

FIG. 78B is a sectional view of the medicament device seen in FIG. 78A;

FIG. 78C is a sectional side view of the medicament device seen in FIG. 78A;

FIG. 79A is a front view of the medicament device seen in FIG. 78A wherein the sheath cap has been removed with the syringe cover contained within the sheath cap;

FIG. 79B is a sectional view of the medicament device seen in FIG. 79A;

FIG. 79C is a sectional side view of the medicament device seen in FIG. 79A;

FIG. 80A is a front view of the medicament device seen in FIG. 78A with both the sheath cap and top cap removed;

FIG. 80B is a sectional view of the medicament device seen in FIG. 80A;

FIG. 80C is a sectional side view of the medicament device seen in FIG. 80A;

FIG. 81A is a front view of the medicament device seen in FIG. 80A wherein the device is in in the process of its administering an injection by a user against an injection site (not shown), illustrating how the sheath is pushed upward within the housing to leave the syringe needle exposed;

FIG. 81B is a sectional view of the medicament device seen in FIG. 80A;

FIG. 81C is a sectional side view of the medicament device seen in FIG. 80A;

FIG. 82A is a front view of the medicament device seen in FIG. 81A wherein the plunger has been pushed by the user and the medication in the barrel of the syringe injected into the injection site;

FIG. 82B is a sectional view of the medicament device seen in FIG. 82A;

FIG. 82C is a sectional side view of the medicament device seen in FIG. 82A;

FIG. 83A is a front view of the medicament device seen in FIG. 82A wherein the medication has been fully injected and the sheath actuated to fall back in place around the syringe needle;

FIG. 83B is a sectional view of the medicament device seen in FIG. 83A;

FIG. 83C is a sectional side view of the medicament device seen in FIG. 83A;

FIG. 84A is a front view of the medicament device seen in FIG. 83A wherein the sheath has been rotated to lock the sheath securely in place around the syringe needle;

FIG. 84B is a sectional view of the medicament device seen in FIG. 84A; and

FIG. 84C is a sectional side view of the medicament device seen in FIG. 84A.

### DETAILED DESCRIPTION

For safety and accuracy, injection by syringe requires skilled technique with formal training. Steps include uncapping the needle by removing the protective needle cover, piercing the membrane of a vial to draw medicament there within, aligning the plunger tip with respective indices to measure a required dose, correctly inserting the needle into a proper injection site, squeezing the syringe plunger while keeping the syringe stable, and recapping the bio-hazardous needle. Issues with injection by syringe include aspiring incorrect dosage, lack of safe portability, tricky syringe manipulation in administering shot, accidental needle sticks, and unsuitable syringe disposal.

Related devices that automate injection include what is commonly referred to as an auto-injector. A conventional auto-injector contains a custom, spring-loaded and pre-filled syringe, which, when triggered by pressured contact, releases a spring-loaded needle into the injection site, squirting in the dose of medicament. Issues with auto-injectors include confusing labeling, unlocking of any safety mechanism, determining where on the device the needle will exit, and accidentally firing the device into a finger by touching the actuator tip.

Accordingly, a medicament device solving these and other problems is desired.

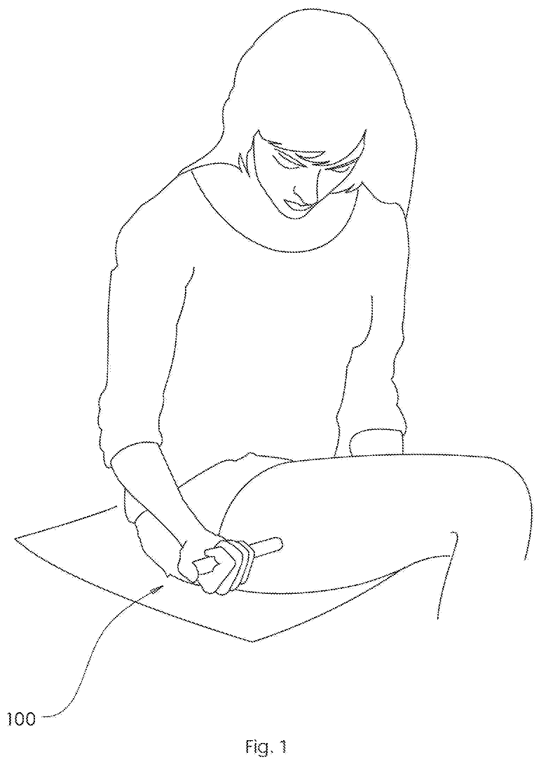

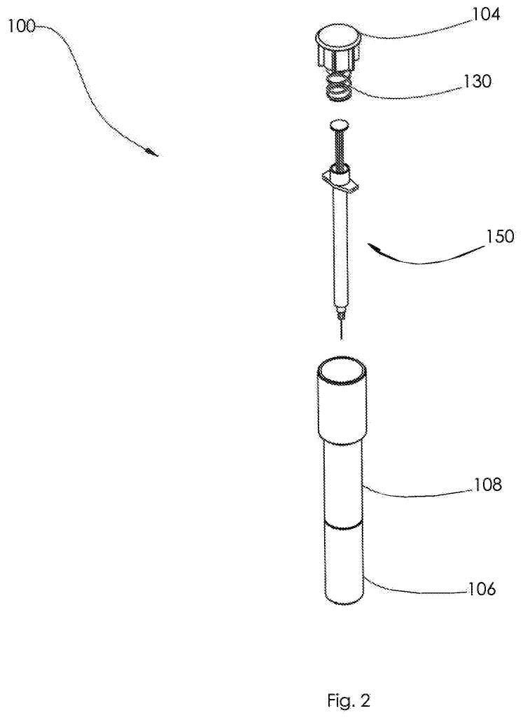

FIGS. 1-12B illustrate an embodiment of a device 100 for administering medicament using a conventional pre-filled syringe 150 such as 1 ml Becton Dickinson tuberculin syringe filled with epinephrine, or such as a common safety syringe. Syringe 150 may comprise barrel 152, plunger 156 that slides within barrel 152, and needle 158. The barrel 152 typically has flanges 154 at one end that aid in pushing the plunger 156 through the barrel 152 containing medicament (not shown). Depending on the intended use of device 100, the medicament may be injected into a site 180 such as a deltoid, triceps, quadriceps or the like of a user or patient.

Medicament device 100 preferably comprises a body assembly 102, a sheath cap 108 at one end of the assembly 102, and a plunger spring 130 and housing cap 104 at the other end of the assembly 102.

Body assembly 102 may be comprised of a housing 108 containing sheath 110, locking mechanism 112, sheath clip 118, and sheath spring 114. The locking mechanism 112 preferably comprises a sheath lock 116. The sheath clip 118 disposed in an annular groove within housing 108. The sheath clip 118 retains the needle sheath 110 during handling of the device 100 until needle 158 insertion.

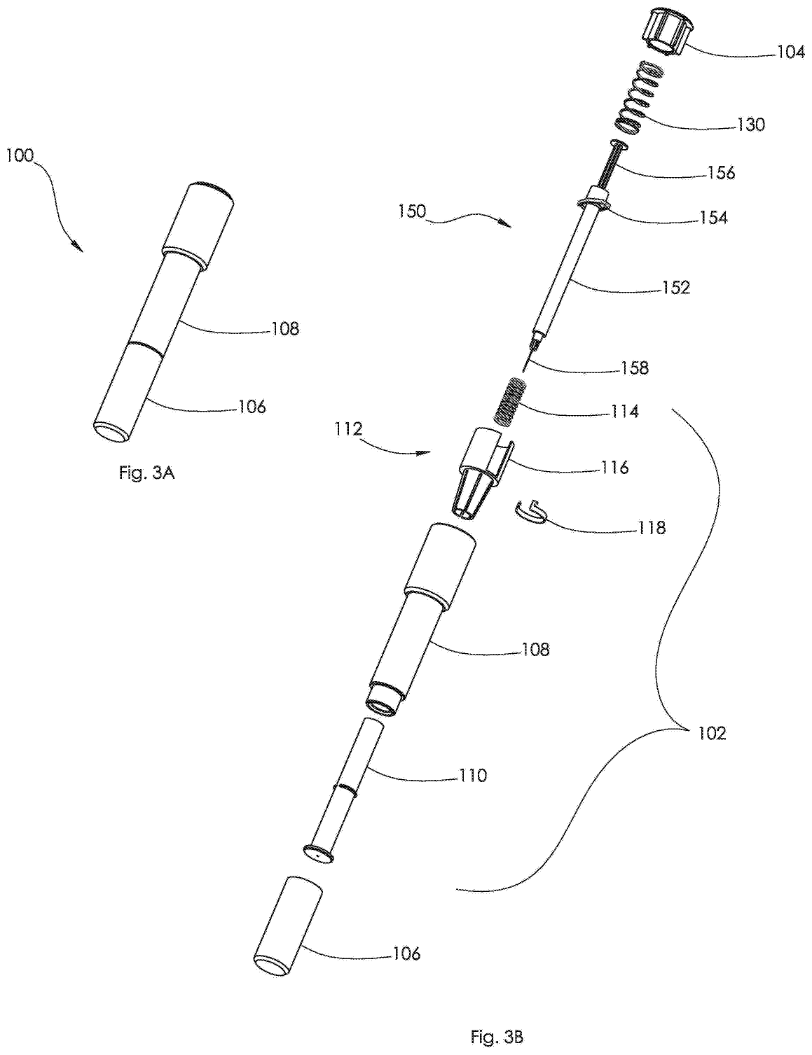

As best seen in FIG. 3B, housing 108 is generally tubular in shape with a top and an opposing bottom. The housing 108 may have a generally wider opening at the top compared to the bottom to be able to receive a conventional syringe 150 placed into the housing 108 from the top. The overall length of the housing 108 is about the same length of barrel 152 of syringe 150 158. Similarly, the overall length of sheath 110 is also about the same length of barrel 152 of syringe 150.

The top of housing 108 is also configured to receive the sheath lock 116. The sheath lock 116 itself has an opening at one end with a slot therein configured to receive and frictionally engage the flanges 154 of syringe 150.

The bottom of the housing 108 is configured to receive the sheath 110 wherein the sheath 110 may freely slide within the housing 108. Sheath lock 116 is not permitted to slide within housing 108 but instead is held firmly in place within housing 108.

When assembled, the entire syringe 150 with its needle 158 is encased within the device 100. Sheath cap 106 and sheath 110 surround the needle 158 when the device 100 is not in use. Housing cap 104 seals the top of the housing 108, wherein one end of plunger spring 130 abuts cap 104 while the other end of spring 130 abuts the flanges 154 of the syringe 150.

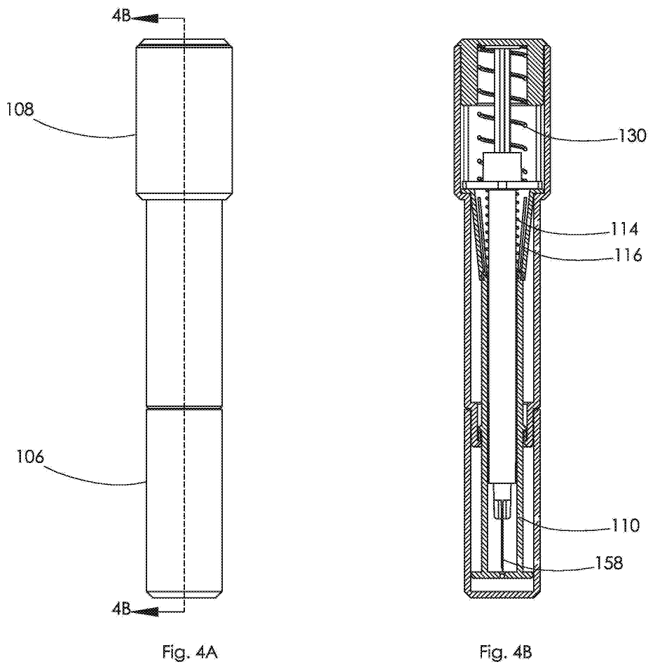

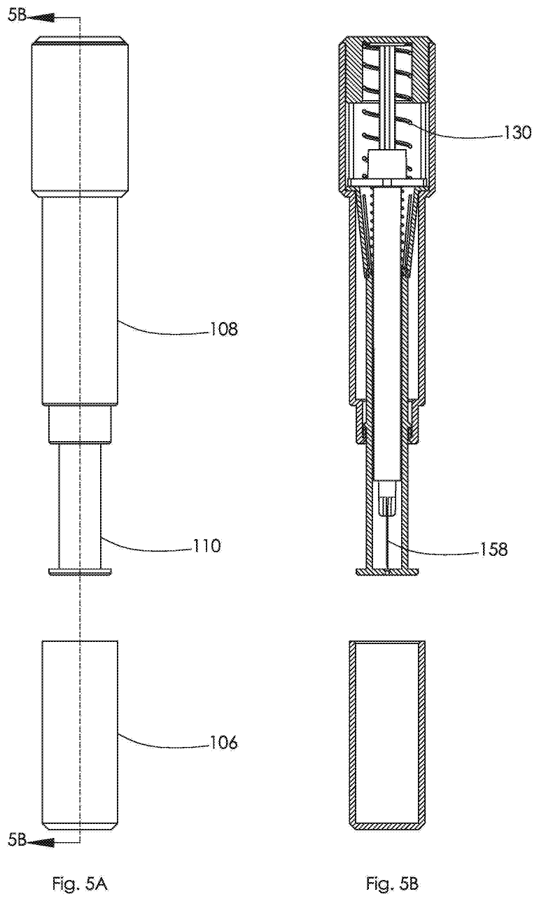

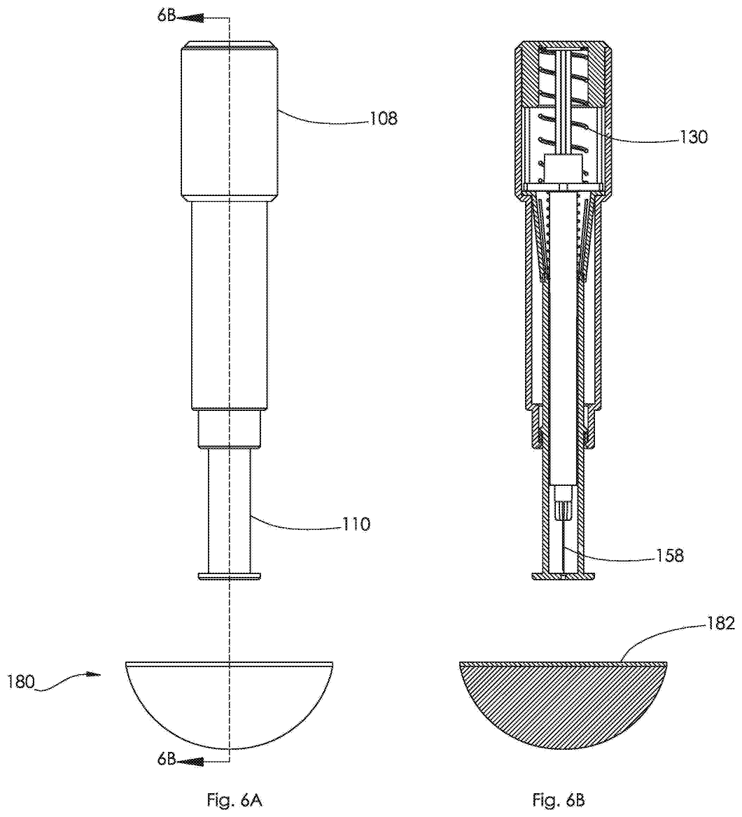

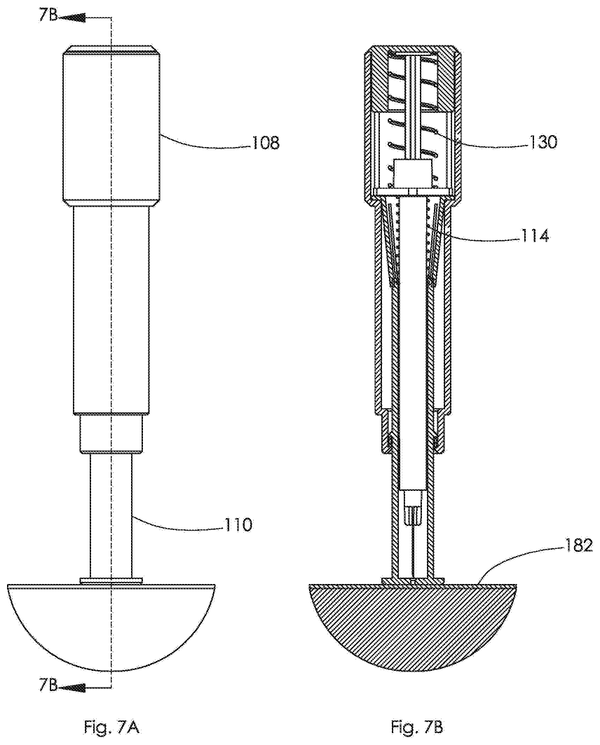

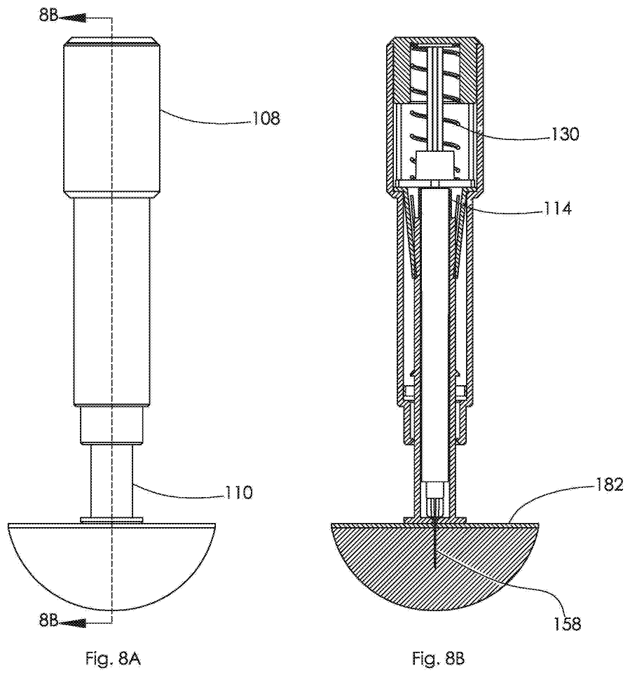

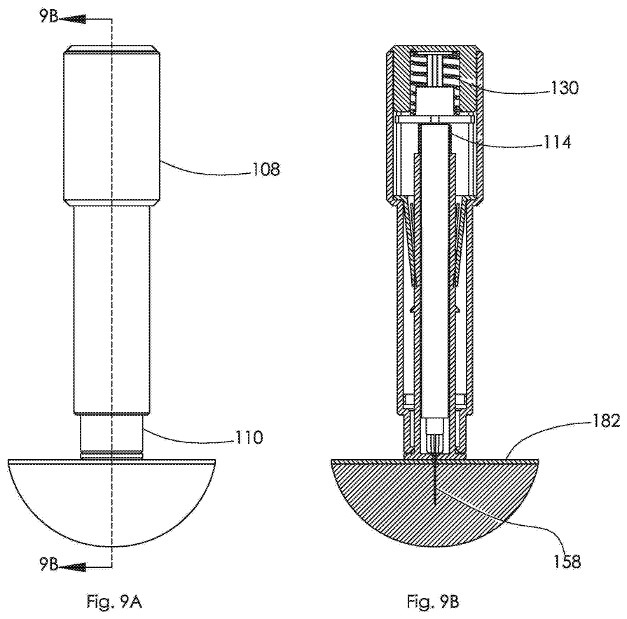

FIGS. 4A-12B show the medicament device 100 in various stages of use. FIGS. 4A and 4B respectively show a front and sectional view of the device 100 fully assembled and capped by cap 106 for safe storage and transportation. FIGS. 5A and 5B respectively show a front and sectional view of the device 100 with cap 106 removed. FIGS. 6A and 6B respectively show a front and sectional view of the device 100 with cap 106 removed, wherein the sheath 110 is disposed directly above a site 180 to be injected once the needle 158 passes through skin 182. FIGS. 7A and 7B respectively show a front and sectional view of the device 100 with cap 106 removed, wherein sheath 110 is touching site 180 to be injected. FIGS. 8A and 8B respectively show a front and sectional view of the device 100 with cap 106 removed, wherein the needle 158 is deployed and passing through skin 182 and sheath spring 114 can be seen compressed. FIGS. 9A and 9B respectively show a front and sectional view of the device 100 with cap 106 removed to deliver medicament to the site 180 and produce an audible clip from the sheath lock 112 snapping past the end of the needle sheath 110. Both sheath spring 114 and plunger spring 130 can be seen compressed in FIG. 9.

FIGS. 10A and 10B respectively show a front view and sectional view of the device 100 with cap removed, wherein the device 100 is in a partially released state after the medicament contained in the syringe 150 has been injected into the site 180. FIGS. 11A and 11B respectively show a front view and sectional view of the device 100 with cap removed, wherein the device 100 is in a fully released state after the medicament contained in the syringe 150 has been injected into the site 180. FIGS. 12A and 12B respectively show a front view and sectional view of the device 100 with cap removed, wherein the device 100 is disposed above the site 180 after injection.

In typical operation, the cap assembly 106 is removed and the sheath 110 is placed against an intended injection site 180. The user then pushes down on the device 100. Continued pushing causes spring-loaded sheath 110 to retract into the housing 108, progressively exposing the syringe needle 158 until completely inserted into the injection site 180. As the stroke is continued beyond the full insertion of the needle 158, the proximal edge of the spring-loaded sheath 110 contacts the underside of the flanges 154 of the syringe barrel 152. Because the movement of the barrel 152 is constrained by the housing 108, the plunger 156 moves within its stationary barrel 152, expelling medicament through the needle 158. The sheath spring 114 requires less compression force than the syringe plunger spring 130, the effect being that the primary stroke lets the needle 158 pierce the skin 182 while the needle sheath 110 retracts. The secondary and culminating stroke actuates the plunger 156, effectively dispensing the bolus of medicament only when the needle 158 is fully inserted into the skin 182. In addition to feeling the device 100 bottom out, the user feels and hears a click confirming stroke completion. The shot having been administered, the user stops pushing down on the device 100 whereby the needle 158 is withdrawn as the spring-loaded sheath 110 extends and locks with a second click upon full extension preventing further use of the device 110. Thus, the sheath lock 116 retains the needle sheath 110 in an extended position that covers the needle 158 of the syringe 150 after injection is complete.

FIGS. 13-21 illustrate another embodiment of a device 200 for administering a medicament such as by an injection using a conventional pre-filled syringe 250. Such a syringe 250 may comprise a barrel 252, plunger 256 that slides within barrel 252, and needle 258. The barrel 252 typically has flanges 254 at one end that aid in pushing the plunger 256 through the barrel 252 containing medicament (not shown) to be administered by a user or patient.

Medicament device 200 preferably comprises a body assembly 202, a sheath cap 206 at end of the assembly 202, and a housing cap 204 with its optional label 224 at the other end of the assembly 202.

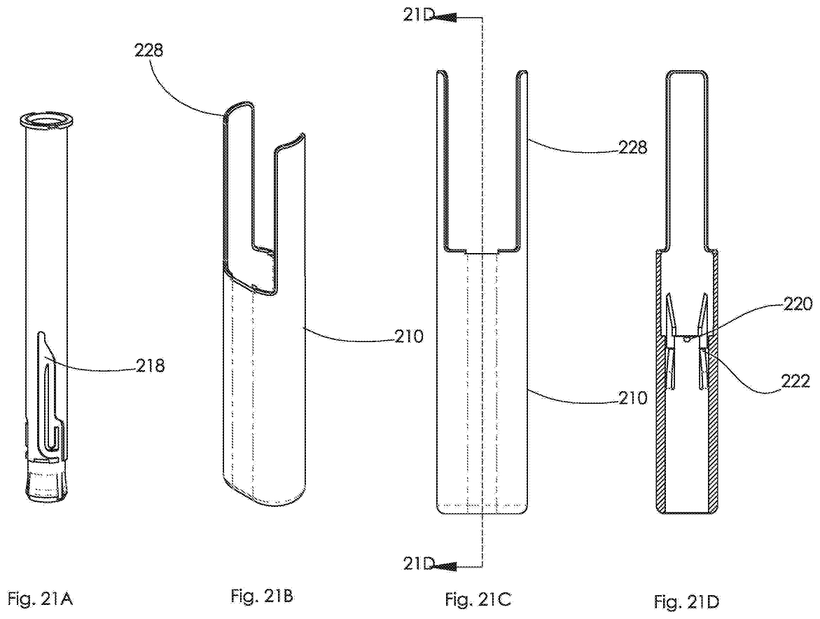

Body assembly 202 may be comprised of a housing 208 containing sheath 210 with its optional sheath seal 226, locking mechanism 212, and sheath spring 214. The locking assembly 212 preferably comprises a sleeve 216 having sleeve tracks 218 configured to receive one or more sheath pins 220 protruding from the interior of sheath 210, as well as sheath snaps 222 disposed about the interior of sheath 210 and protruding therefrom, as illustrated in FIGS. 21A-21D. Sheath seal 226 may serve to seal the chamber of syringe 250 until pierced by the needle 258.

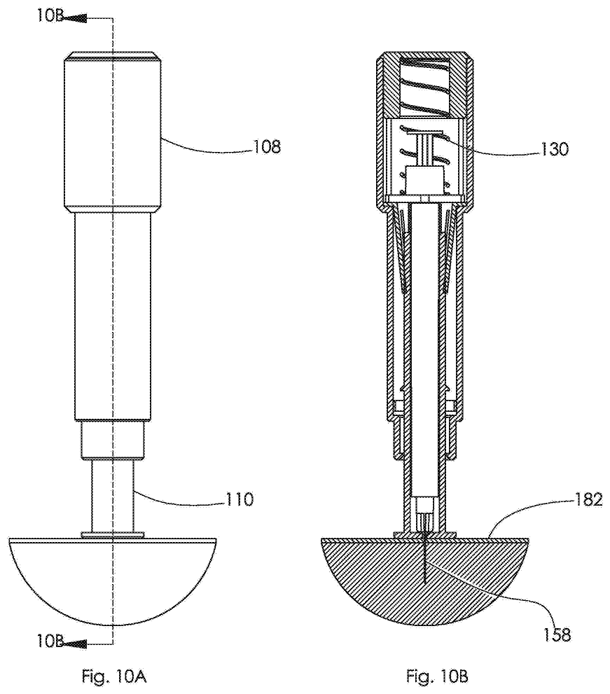

As best seen in FIG. 14B, housing 208 is generally tubular in shape with a top and an opposing bottom. The top of housing 208 is configured to receive and frictionally engage a conventional syringe 250 placed into the housing 208 from the top. The top of housing 208 is also configured to receive the sleeve 216 having an opening at one end configured to receive syringe 250. The bottom of the housing 208 is configured to receive the sheath 210 wherein the sheath 210 may freely slide within the housing 208.

The overall length of the housing 208 is about the same length of the barrel 252 of conventional syringe 250. Similarly, the overall length of sheath 210 with its sheath fingers 228 is also about the same length of the barrel 252.

When assembled, the entire syringe 250 with its needle 258 is encased within the device 200. Sheath cap 206 and sheath 210 surround the needle 258 when the device 200 is not in use. Housing cap 204 seals the top of the housing 208.

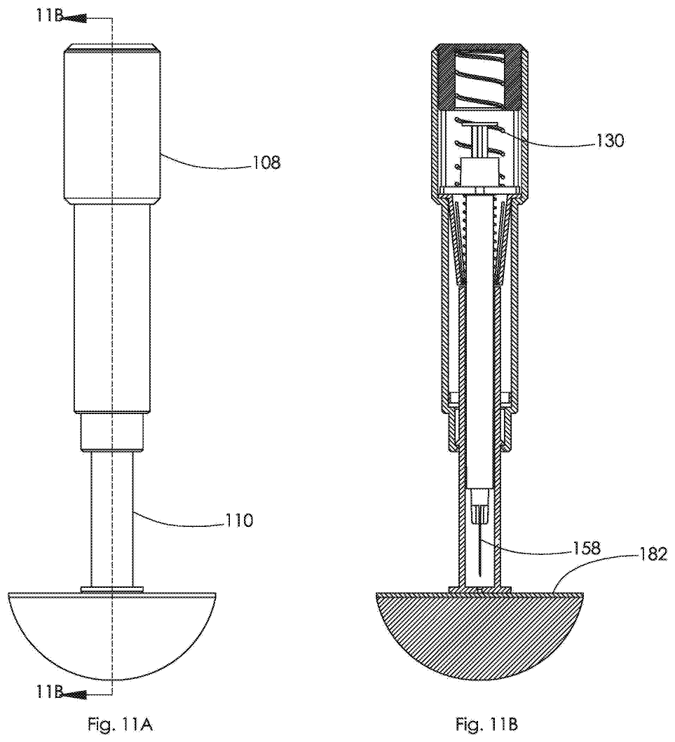

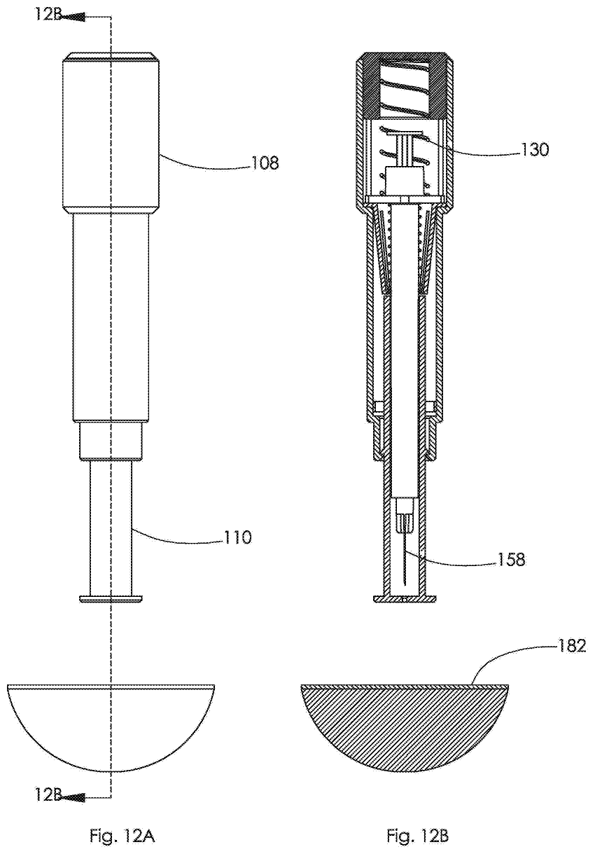

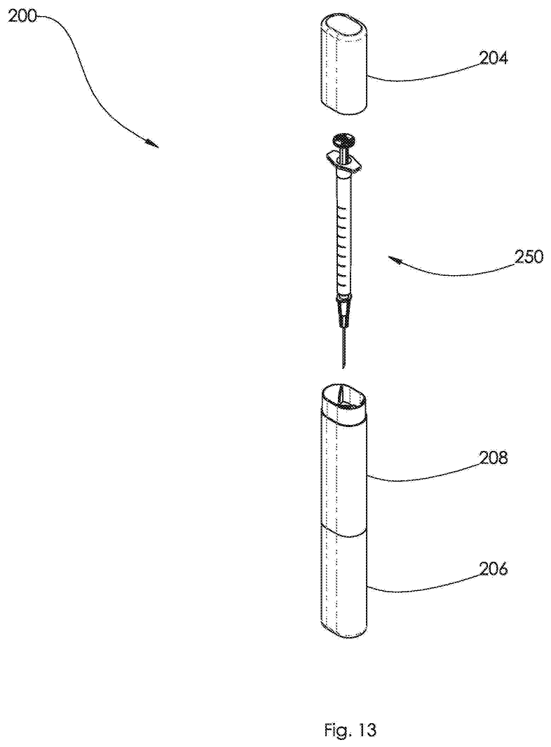

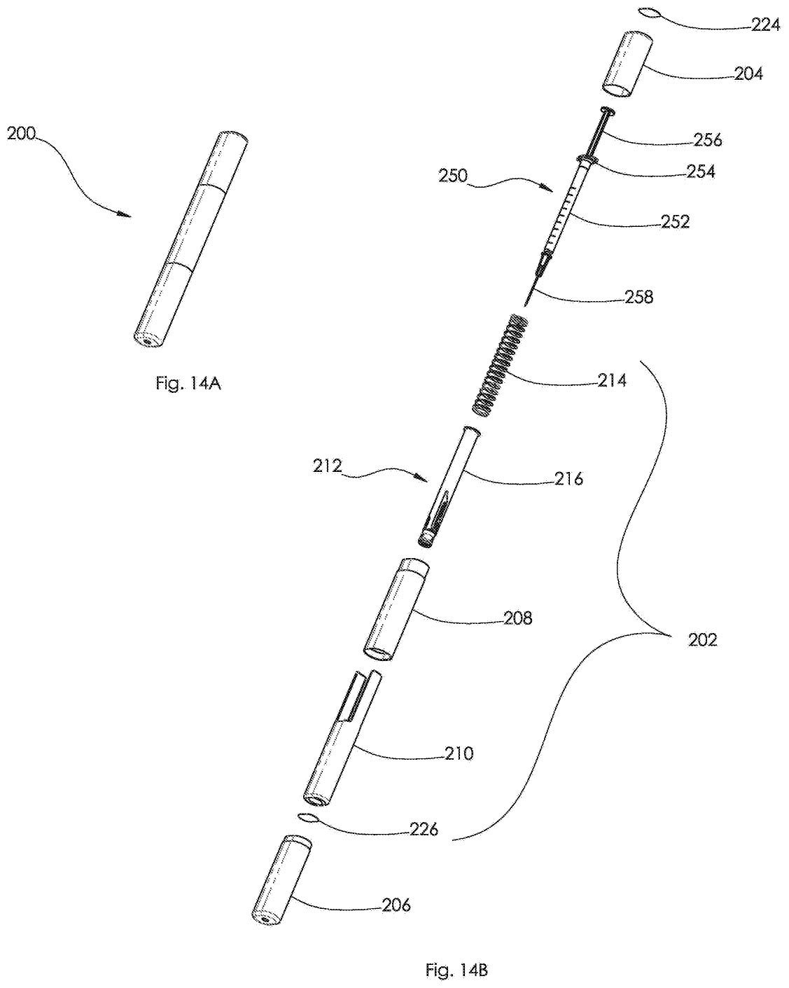

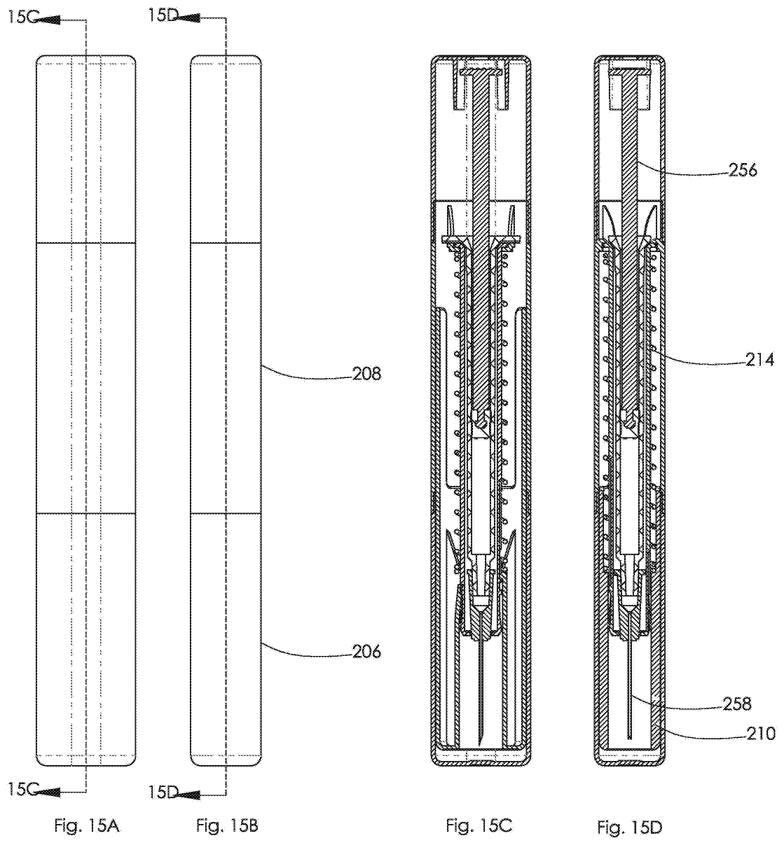

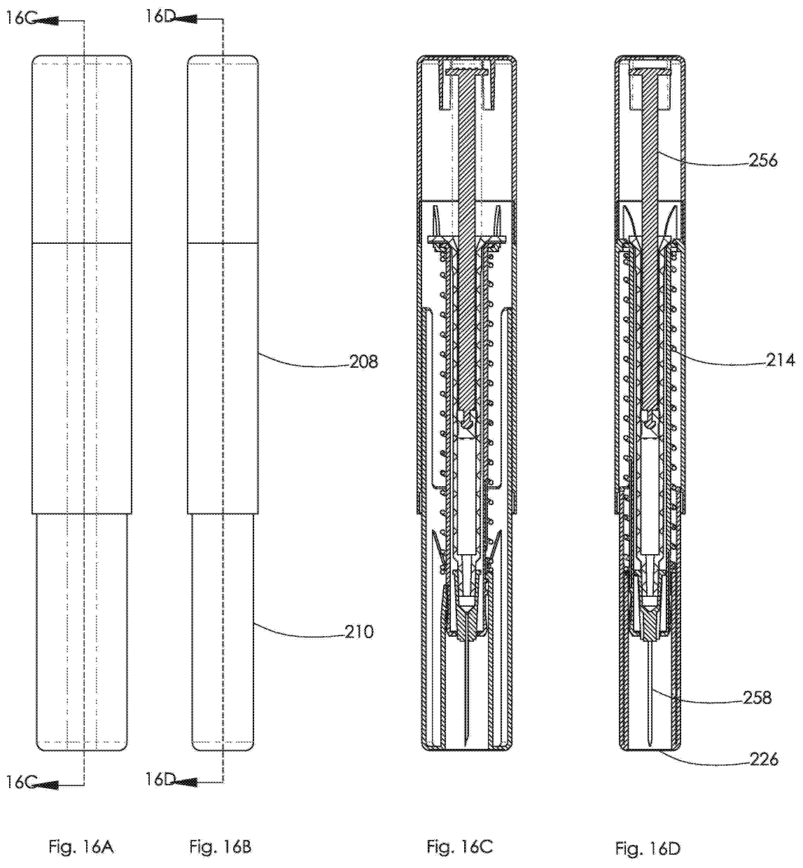

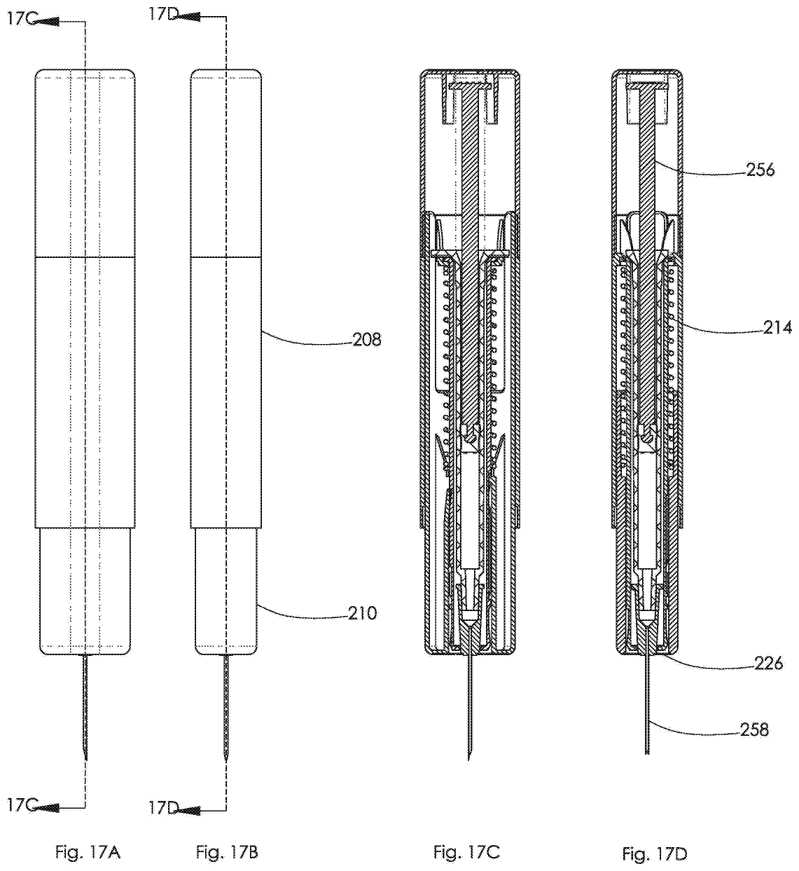

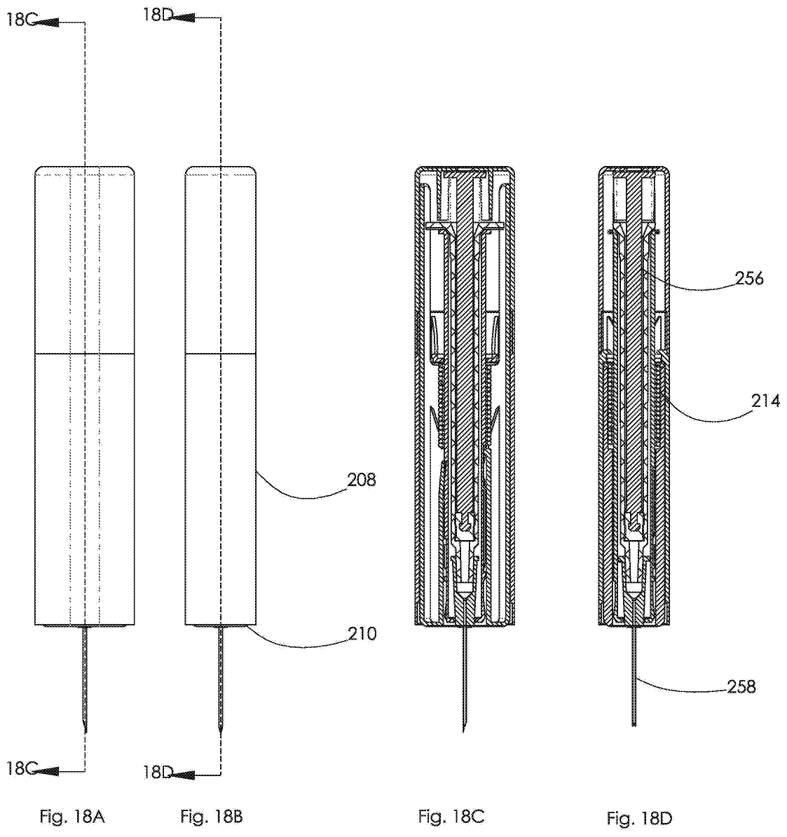

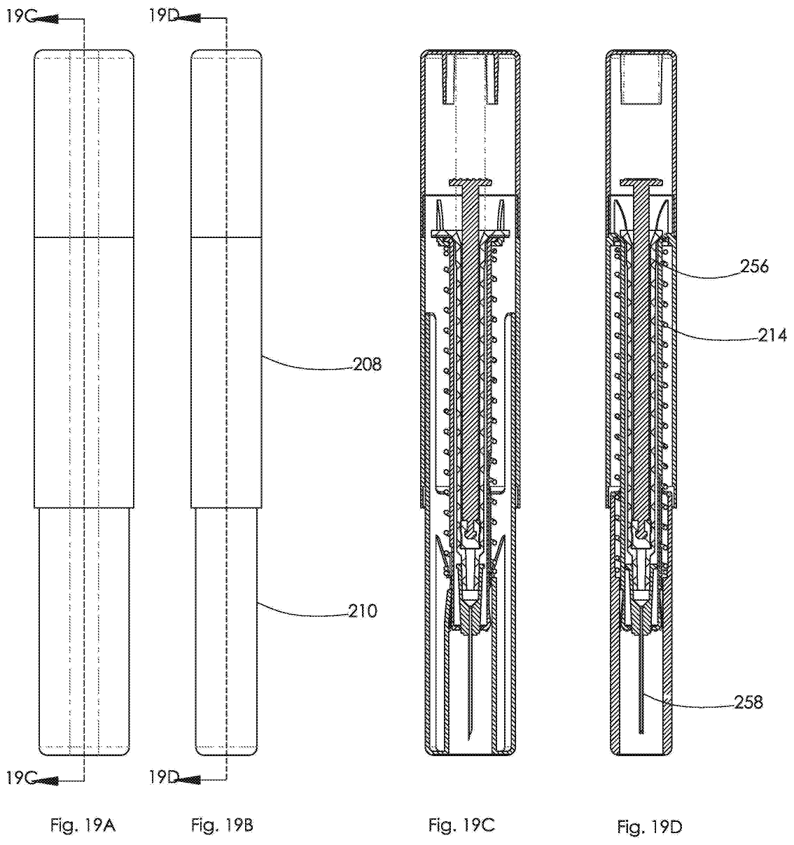

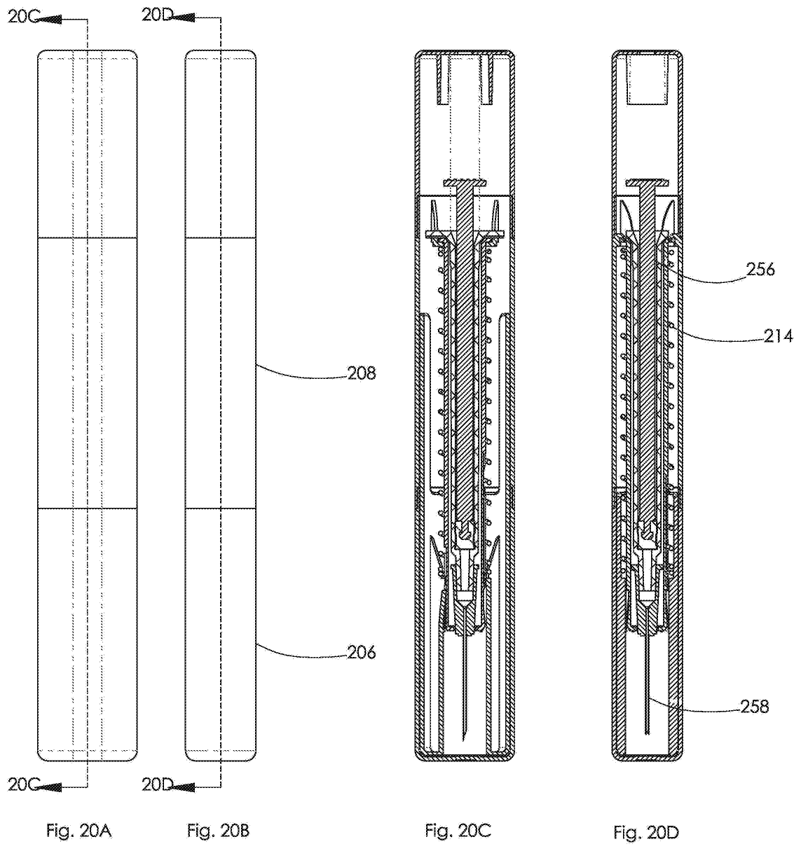

FIGS. 15A-20D show the medicament device 200 in various stages of use. FIGS. 15A-15D show the device 200 fully assembled and capped by cap 206 for safe storage and transportation. FIGS. 16A-16D show the device 200 with cap 206 removed. FIGS. 17A-17D show the device 200 in a stage whereby the needle 258 is deployed to deliver medicament. FIGS. 18A-18D show the device 200 with spring 214 in a compressed state wherein medicament has been delivered or is being delivered. FIGS. 19A-19D show the device 200 in a fully released state after the medicament has been delivered. FIGS. 20A-20D show the device 200 in a fully released and locked state with the device being recapped for disposal.

In typical operation, the cap assembly 206 is removed and the sheath 210 is then placed against an intended injection site. The user then pushes down on the device 200. Continued pushing causes spring-loaded sheath 210 to retract into the housing 208, progressively exposing the syringe needle 258 until it is completely inserted into the injection site, fully compressing sheath 210 wherein sheath fingers 228 slide within housing body 208. Concurrently, sheath pins 220 found on the internal surface of sliding sheath 210 track in sleeve tracks 218, effectively guiding and rotating the sleeve 216 concentrically as the sheath 210 draws in and into bifurcated sleeve tracks 218.

When sheath 210 is fully compressed into body 208, sheath fingers 228 contact and spread snaps 222 to permit the plunger 256 to slide within barrel 252 and thereby expel medicament through syringe needle 258.

The shot having been administered, the device 200 is then removed from the injection site, withdrawing syringe needle 258 and allowing spring-loaded sheath 210 to re-extend from the body 208 due to the biasing force of sheath spring 214. Sheath pins 220 track in the straight and slightly longer bifurcation found in the now-rotated sleeve tracks 218. At full extension, sheath 210 bottoms out at a position slightly more extended than its initial position, thus allowing sheath snaps 222 to engage and lock out sheath 210 for the purpose of protecting syringe needle 258, thereby preventing further use of the device 200.

The disclosed inventions advantageously may use a conventional syringe pre-loaded by the user for pre-filled, portable injections (e.g., insulin, epinephrine). The device 100, 200 may be produced in varying lengths and sizes to accommodate different syringe sizes (e.g., 0.3 mL, 1 mL, 3 mL). The device 100, 200 may also be produced in a length that precludes over-filling the syringe beyond a set dosage, and thus avoid a potential overdose.

The disclosed inventions may also function with any prescribed quantity of medicament equal to or less than full capacity. For example, the device 100, 200 may be adjusted to dispense varying doses of medicament by means of a coarsely threaded cap, which may be adjusted to vary the travel length of syringe plunger, with indices showing the relative amount of medicament to be dispensed.

The housing of the device 100, 200 may also include a window to provide a direct view of the medication. Such a window may be circumscribed with a color that, when matched with the medicament color as viewed through said window, indicates the medicament has expired. In such a situation, the syringe may advantageously be changed out for a new syringe with non-expired medicament.

The sheath may be marked with color or graphics to let the user know the syringe needle is fully inserted (triggering medicament injection). In the alternative, a battery-powered light emitting diode or the like may illuminate to indicate the syringe needle is fully inserted.

The housing may be made of a transparent material that reveals the inner functioning of the device, including revealing the amount of medicament in syringe. The housing may also incorporate instructional graphics.

The device 100, 200 may include a peel-off-cover antiseptic pad for the purpose of cleaning the injection site. Such a pad may be adhered to the housing cap, for example.

The housing cap may permanently lock when inserted into housing, sealing in the syringe to assure single-use only. In the alternative, the housing cap may be removable to change out the syringe as desired so the device may be further used. If desired to limit reuse of device 100, 200, then the housing cap may be removable by means of a special tool.

The device 100, 200 may be made of disposable, single-use materials, or alternatively, made of reusable materials that could withstand repeated sterilization. Regardless of the materials used, the device 100, 200 may incorporate a peel tab or breakaway part that renders the device non-functional after a single use, insuring single-use only.

The medical device 100, 200 may also include a split thrust washer located between the syringe flanges and the housing cap that deforms when squeezed to provide a tactile and audible click when the plunger is fully depressed, letting the user know that the stroke is complete, all medicament is dispensed, and the shot has been successfully administered.

The device 100, 200 may integrate a voice-chip to provide audible instructions and status such as when a medicament has expired or is nearing the expiration date. Similarly, the device 100, 200 may integrate a chemical or mechanical timer to indicate when a determined number of days have passed since syringe filling, indicating expiration of contents. Such a timer may be activated when the housing cap is closed to begin the countdown to medicament expiration.

The device 100, 200 may include a shrink-wrap thermoplastic with graphics wherein the wrap is applied to seal and retain the protective cap and then peeled away for use. Similarly, the housing body may be sheathed in shrink-wrap thermoplastic with graphics showing instructional information, expiration date, custom branding, or the like.

The device 100, 200 may include a flat surface on an otherwise cylindrical body to keep the device from rolling. Such a flat surface may be used to print indicia via a felt-tip marker or the like.

The device 100, 200 may include a rotating collar on an otherwise cylindrical body that is positioned by the assembler or user to show the loaded medicament dose. The rotating collar may then be locked into place by virtue of device assembly.

The device 100, 200 may include a metal sleeve within housing, which when pre-chilled with ice or refrigeration, provides cooling for heat-sensitive medicament. In the alternative or in addition thereto, the device 100, 200 may be shaped to fit into a refrigerated chiller to pre-cool heat-sensitive medicament during transportation.

The housing may be configured to contain a plurality of syringes in the event that administration of initial injection is inadequate to treat the present condition (e.g., epinephrine administration for anaphylaxis typically calls for a second dose if symptoms do not abate in a timely manner) or for convenience of having more than one injection at hand (e.g., fertility injections are typically timed hourly).

The device 100, 200 may also be used for oral medication dispensation, concealing a standard syringe and its association with needles.

It should now be apparent that the disclosed inventions have many advantages over conventional devices. Conventional auto-injectors, upon trigger-release, automatically inject needle and medicament, presumably into an intended injected site. Unlike auto-injectors, the disclosed inventions do not automatically insert the needle, an advantage in avoiding accidental actuation that happens with conventional auto-injectors.

Moreover, devices 100, 200 rely on manual action to insert the needle at the injection site, like a standard syringe, making needle sticks less likely than when using an auto-injector, which fires its needle upon contact with any surface, including hands, thumbs, and fingers. The disclosed inventions 100, 200 also inject medicament only after the needle is fully inserted, unlike conventional auto-injectors, which inject medicament immediately upon trigger release. Safety issues, such as a child accidentally firing an adult-dosage auto-injector and thereby receiving twice the recommended amount of medicament for their size, are thus avoided. Also avoided is the dispensing of medicament along the needle tract during insertion, where medicament may not get to its intended target area as may occur with conventional auto-injectors.

The disclosed inventions 100, 200 also facilitate hypodermic injection using an evolved, standard ubiquitous syringe rather than a proprietary or custom syringe that may fail. Moreover, the disclosed inventions 100, 200 may be filled to a prescribed dose of medicament rather than “one-dose-fits-all” as found with auto-injectors and factory-filled syringes. Such pre-loading of medicament may conveniently be done in a domestic setting such as the user's home, in a clinical setting such as a doctor's office, or in an institutional setting such as a school. Indeed, as the contained syringe is pre-filled to a prescribed dosage, the disclosed inventions 100, 200 are ready for use and may be rapidly deployed and used in emergency or military situations.

The sheath cap of the disclosed inventions also acts as both protector and safety cover against accidental activation. Moreover, after use, the locking mechanism of the device 100, 200 protects the needle from being extended from the sheath when the sheath cap is not present, resulting in another advantageous and novel safety feature.

The disclosed inventions 100, 200 are also simpler and safer than administering shots with a standard syringe aspirating from a standard vial. Fortunately, the use of the disclosed inventions is not dissimilar to the customary administering of a typical shot. Such action is in contrast to the violent firing of a needle into skin as is done with a conventional auto-injector. Moreover, because the contained syringe is pre-filled to a prescribed dosage, an unskilled user may quickly and safely administer a shot.

Finally, the disclosed injectors 100, 200 have only two actions to administer a shot: removing the housing cap and pushing down the device on an intended injection site. Conventional auto-injectors require removal from a carrier tube, removal of a locking key, and then pushing onto the intended injection site.

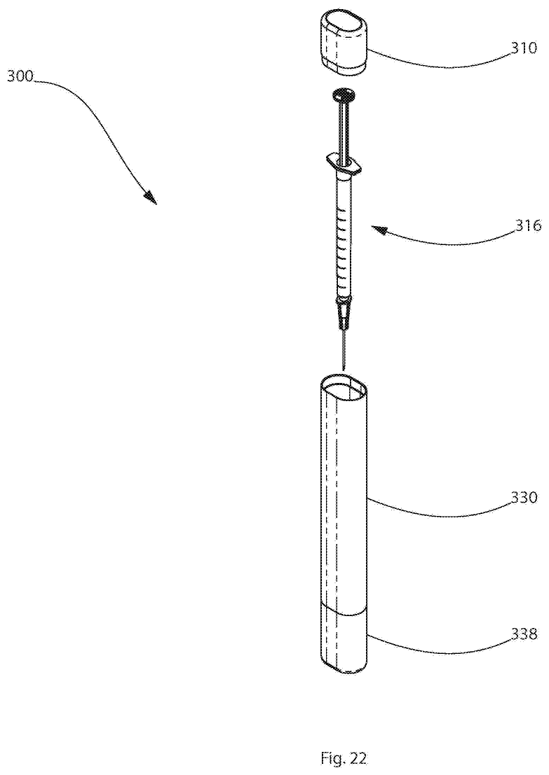

FIGS. 22-30 illustrate another embodiment of a device 300 for administering a shot from a standard pre-filled syringe 316. Such a syringe 316 may comprise a barrel 320, plunger 318 that slides within barrel 320, and needle 324. The barrel 320 typically has flanges 321 at one end that aid in pushing the plunger 318 through the barrel 320 containing medicament (not shown) to be administered by a user or patient.

Medicament device 300 preferably comprises a body assembly 302, a cap assembly 304 at one end of the body assembly 302, and a top assembly 306 at the other end of the body assembly 302 with its optional label 308.

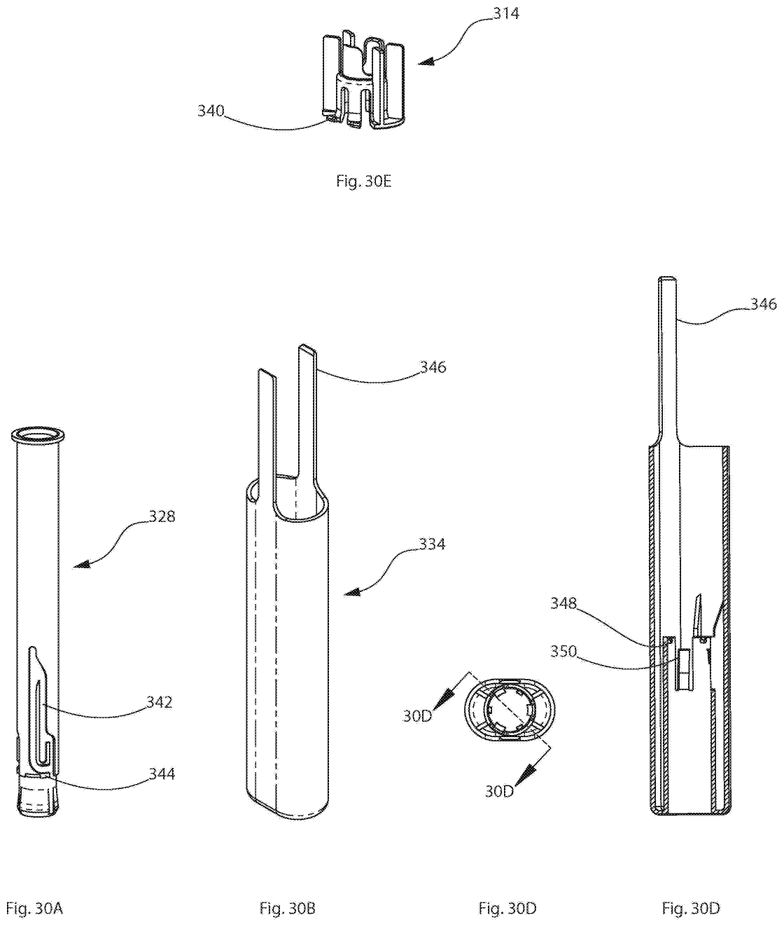

Body assembly 302 may be comprised of a body 330 containing sheath 332 with its optional sheath seal 336, locking mechanism 326, and sheath spring 332. The locking assembly 326 preferably comprises a sleeve 328 having a sleeve ledge 344 and sleeve tracks 342 configured to receive one or more sheath pins 348 protruding from the interior of sheath 334, as well as sheath snaps 350 disposed about the interior of sheath 334 and protruding therefrom, as illustrated in FIGS. 30A-30D. Sheath seal 336 may serve to seal the chamber of syringe 316 until pierced by the needle 324.

Cap assembly 304 may include cap 338. Top assembly 306 may include top 310, pusher spring 312, pusher 314, and top label 308. Sheath spring 332 requires less compression force than the pusher spring 312.

As best seen in FIG. 22, body 330 is generally tubular in shape with a top and an opposing bottom. The top of body 330 is configured to receive and frictionally engage conventional syringe 316 placed into body 330 from the top. The top of body 330 is also configured to receive the sleeve 326 having an opening at one end configured to receive syringe 316. The bottom of the body 330 is configured to receive the sheath 334 wherein the sheath 334 may freely slide within body 330.

The overall length of body 330 is about the same length of the barrel 320 of conventional syringe 316. Similarly, the overall length of sheath 334 with its sheath fingers 340 is also about the same length of the barrel 320.

When assembled, the entire syringe 316 with its needle 324 is encased within the device 300. Sheath cap 338 and sheath 334 surround the needle 316 when the device 300 is not in use. Cap 338 then seals body 330.

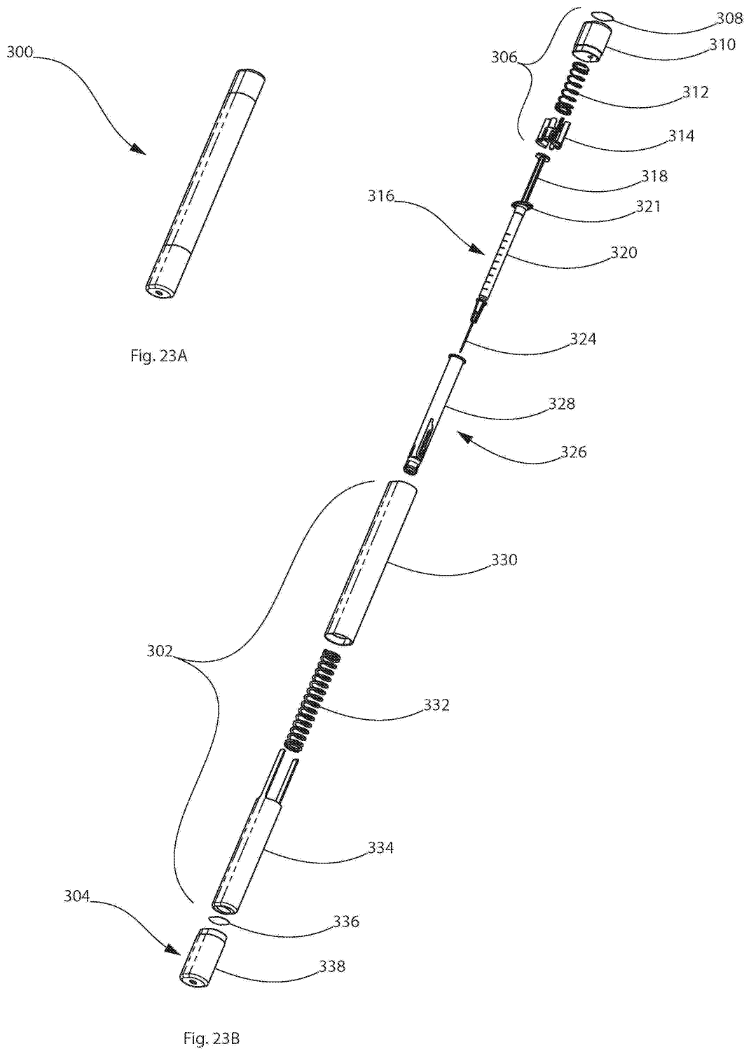

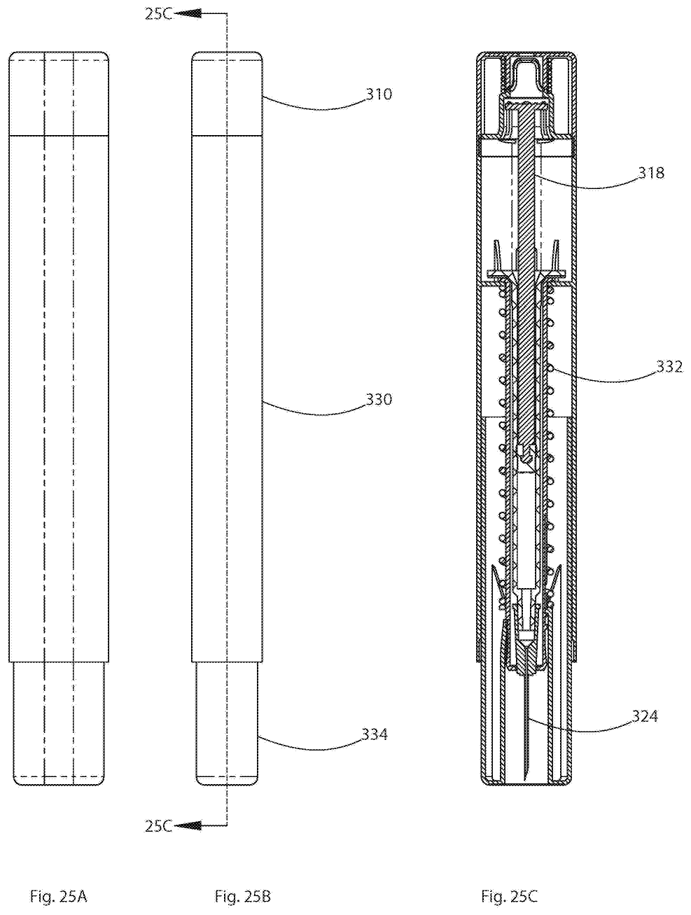

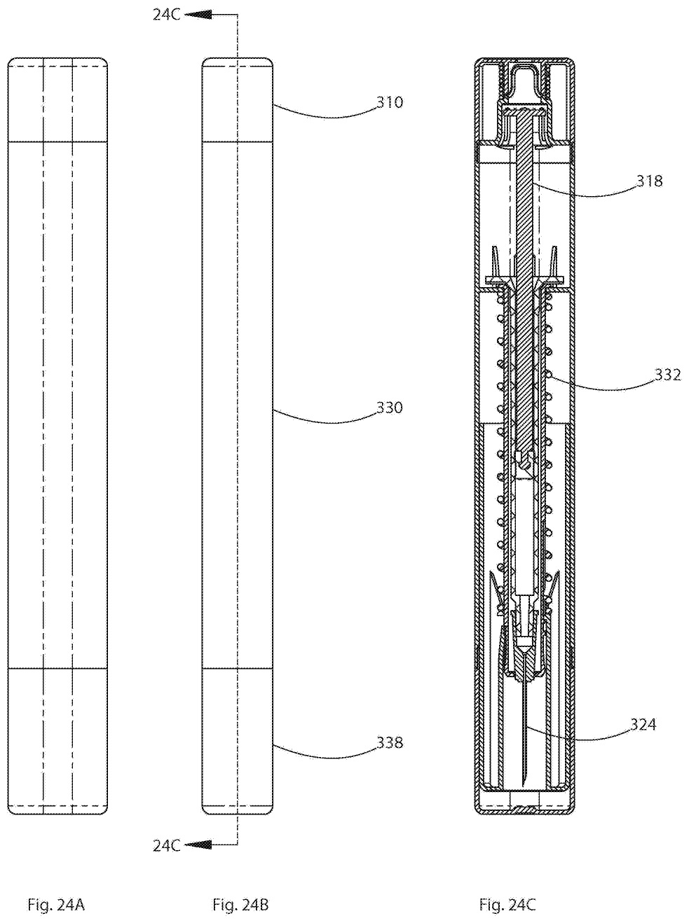

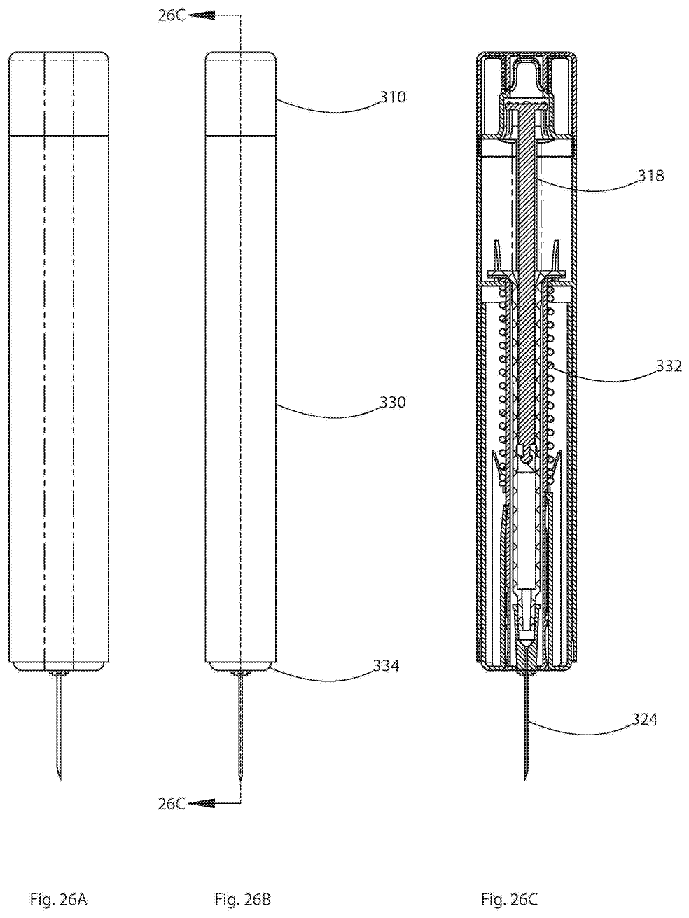

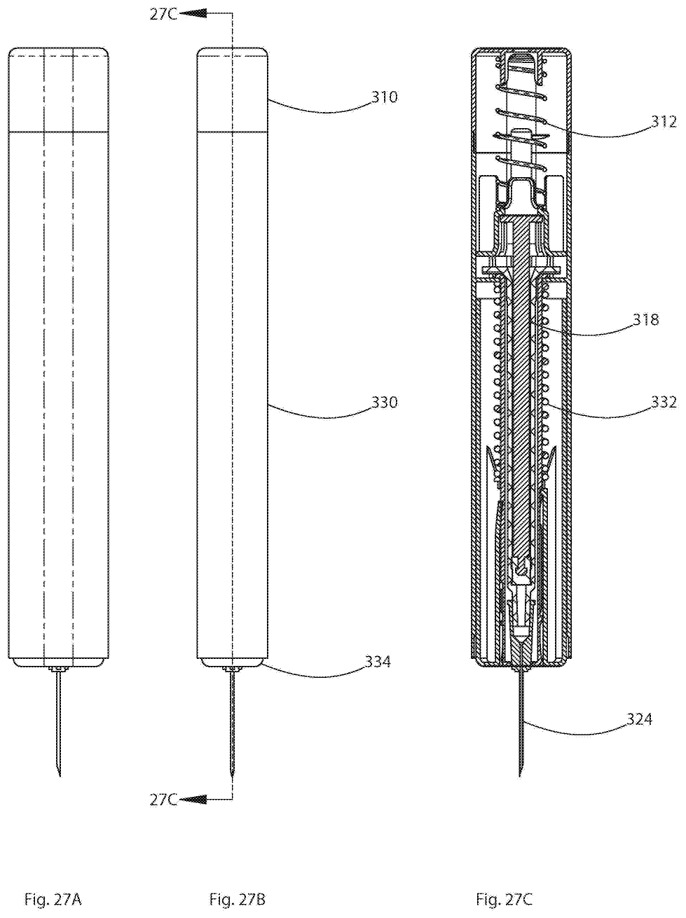

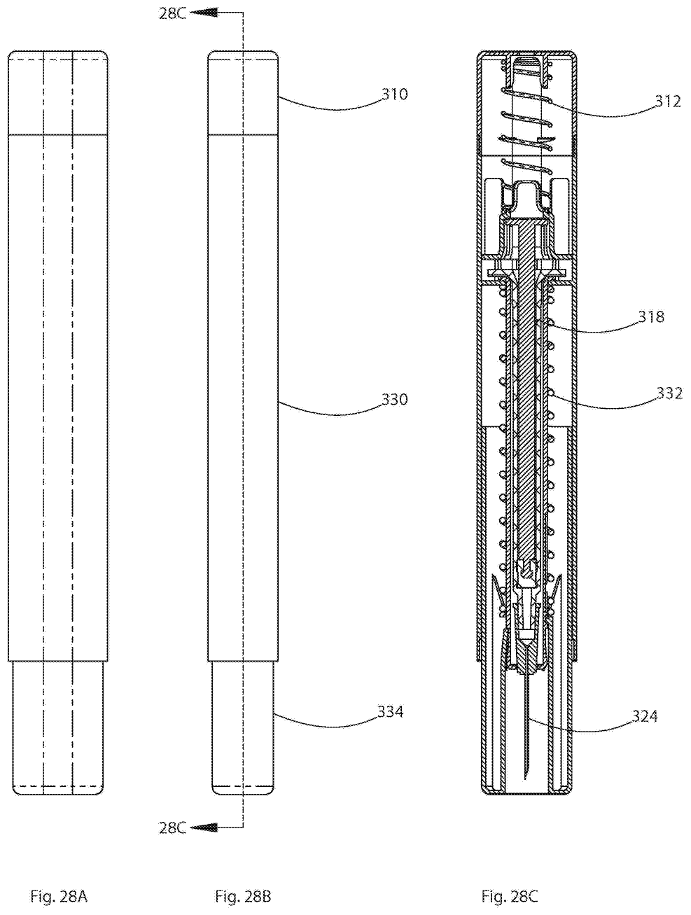

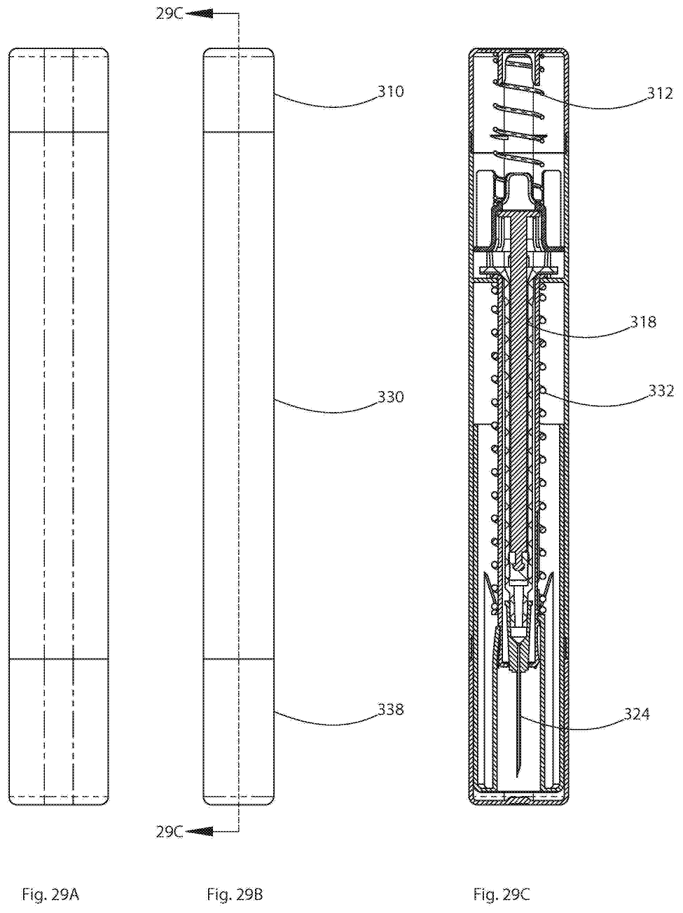

FIGS. 24A-29C show the medicament device 300 in various stages of use. FIGS. 24A-24C show the device 300 fully assembled and capped by cap 338 for safe storage and transportation. FIGS. 25A-25C show the device 300 with cap 338 removed. FIGS. 26A-26C show the device 300 in a stage whereby the needle 324 is deployed to deliver medicament. FIGS. 27A-27C show the device 300 with spring 332 in a compressed state wherein medicament has been delivered or is being delivered. FIGS. 28A-28C show the device 300 in a fully released state after the medicament has been delivered. FIGS. 29A-29C show the device 300 in a fully released and locked state with the device 300 being recapped for disposal.

In typical operation, there are three stages of use: start, insert, and plunge, wherein sheath fingers 346 push snaps 350 releasing pusher 314 that depresses syringe plunger 318 dispensing syringe 316 contents. The user begins by removing the cap assembly 304. The sheath 334 is then placed against an intended injection site (e.g., deltoid, triceps, quadriceps), with the user then pushing down on the device 300. Continued pushing causes spring-loaded sheath 334 to retract into the body 330, progressively exposing the syringe needle 324 until it is completely inserted into the injection site, fully compressing sheath 334 wherein sheath fingers 346 slide within body 330. Concurrently, sheath pins 348 found on the internal surface of sliding sheath 334 track in sleeve tracks 342 are effectively guided by the sliding sleeve 328 concentrically as the sheath 334 draws in, guided by bifurcated sleeve tracks 334, as best seen in FIG. 30A.

When sheath 334 is fully compressed into body 330, sheath fingers 340 contact and spread snaps 350, releasing pusher 314 to slide within the body 330 as driven by the compressed pusher spring 312. Pusher 314 then depresses syringe plunger 318 fully into the syringe barrel 320, expelling the medicament through syringe needle 324.

The shot having been administered, the device 300 is then removed from the injection site, withdrawing syringe needle 324 and allowing spring-loaded sheath 334 to re-extend from the body 330 due to the biasing force of sheath spring 332. Sheath pins 348 track in the straight and slightly longer bifurcation found in the now-rotated sleeve tracks 342. At full extension, sheath 334 bottoms out at a position slightly more extended than its initial position, thus allowing sheath snaps 350 to engage and lock out sheath 334 for the purpose of protecting syringe needle 324, thereby preventing further use of the device 300.

The syringe 316 of the device 300 may be pre-loaded by the user for pre-filled, portable injections (e.g., insulin, epinephrine). The device 300 may be produced in varying lengths and sizes to accommodate different syringe sizes (e.g., 0.3 mL, 1 mL, 3 mL). The device 300 may also be adjusted to dispense varying doses of medicament by means of a coarsely threaded cap assembly 304, which may be adjusted to vary the travel length of syringe plunger 318, with indices showing the relative amount of medicament to be dispensed. The body 330 may also include a window to provide a direct view of the medication. Such a window may be circumscribed with a color that, when matched with the medicament color as viewed through said window, indicates the medicament has expired. In such a situation, the syringe 316 may advantageously be changed out for a new syringe with non-expired medicament.

The sheath 334 may be marked with color or graphics to let the user know the syringe needle 324 is fully inserted (triggering medicament injection). In the alternative, a battery-powered light emitting diode or the like may illuminate to indicate the syringe needle 324 is fully inserted. The sleeve 328 may also be marked with color or graphics visible through a window in the body 330 to let the user know injection is complete.

The body 330 may be made of a transparent material that reveals the inner functioning of the device 300, including revealing the amount of medicament in syringe 316. The body 330 may alternatively be made of transparent or translucent material and wrapped with an opaque label in a manner that reveals the medicament level in syringe 316 through a label window. The body 330 may similarly incorporate instructional graphics.

The device 300 may include a peel-off-cover antiseptic pad for the purpose of cleaning the injection site. Such a pad may be adhered to the top or cap assemblies, 306, 304, for example.

The top assembly 306 may permanently lock when inserted into body 330, sealing in syringe 316 to assure single-use only. In the alternative, the top assembly 306 may be removable to change out syringe 316 as desired so the device 300 may be further used. If desired to limit device 300 reuse, the top assembly 306 may be removable by means of a special tool.

The cap 338 may be press-fit onto body 330 for removal by twisting off. Cap 338 may be press-fit onto body 330 with a cam feature to facilitate removal as it is twisted. The cap 338 may also include an attachment point for a lanyard. The cap 338 may also be integrated with a label that covers the whole device 300, with scoring marks to tear off the portion of the label that is acting as a cap. Similarly, the amount and/or type of medicament may be printed graphically on the proximal body tip.

Sheath 346 may lock upon extension after the needle 324 is withdrawn and then unlocked by a tool such as a pick, paper clip, or small screwdriver.

The device 300 may be made of disposable, single-use materials, or alternatively, made of reusable materials that could withstand repeated sterilization. Regardless of the materials used, device 300 may incorporate a peel tab or breakaway part that renders the device 300 non-functional after a single use, insuring single-use only.

The device 300 may also include a split thrust washer located between the syringe flanges 321 and the body cap 338 that deforms when squeezed to provide a tactile and audible click when the plunger 318 is fully-depressed, letting the user know that the stroke is complete, all medicament is dispensed, and the shot has been successfully administered.

The device 300 may integrate a voice-chip to provide audible instructions and status such as when a medicament has expired or is nearing the expiration date. Similarly, the device 300 may integrate a chemical or mechanical timer to indicate when a determined number of days have passed since syringe filling, indicating expiration of contents. Such a timer may be activated when top assembly 306 is closed to begin the countdown to medicament expiration.

The device 300 may include a shrink-wrap thermoplastic with graphics wherein the wrap is applied to seal and retain the protective cap 338 and then peeled away for use. Similarly, the body 330 may be sheathed in shrink-wrap thermoplastic with graphics showing instructional information, expiration date, custom branding, or the like.

The device 300 may include a flat surface on an otherwise cylindrical body 330 to keep the device 300 from rolling. Such a flat surface may be used to print indicia via a felt-tip marker or the like.

The device 300 may further include a rotating collar on an otherwise cylindrical body 330 that is positioned by the assembler or user to show the loaded medicament dose. The rotating collar may then be locked into place by virtue of device assembly.

The device 300 may include a metal sleeve 328 within body 330, which when pre-chilled with ice or refrigeration, provides cooling for heat-sensitive medicament. In the alternative or in addition thereto, the device 300 may be shaped to fit into a refrigerated chiller to pre-cool heat-sensitive medicament during transportation.

The body 330 may be configured to contain a plurality of syringes in the event that administration of initial injection is inadequate to treat the present condition (e.g., epinephrine administration for anaphylaxis typically calls for a second dose if symptoms do not abate in a timely manner) or for convenience of having more than one injection at hand (e.g., fertility injections are typically timed hourly).

The device 300 may also be used for oral medication dispensation, concealing a standard syringe and its association with needles.

The device 300 may incorporate a microchip, such as a radio-frequency identification or near-field communication chip, for geolocation, product information, expiration information, or other such status of the device 300 and its contents.

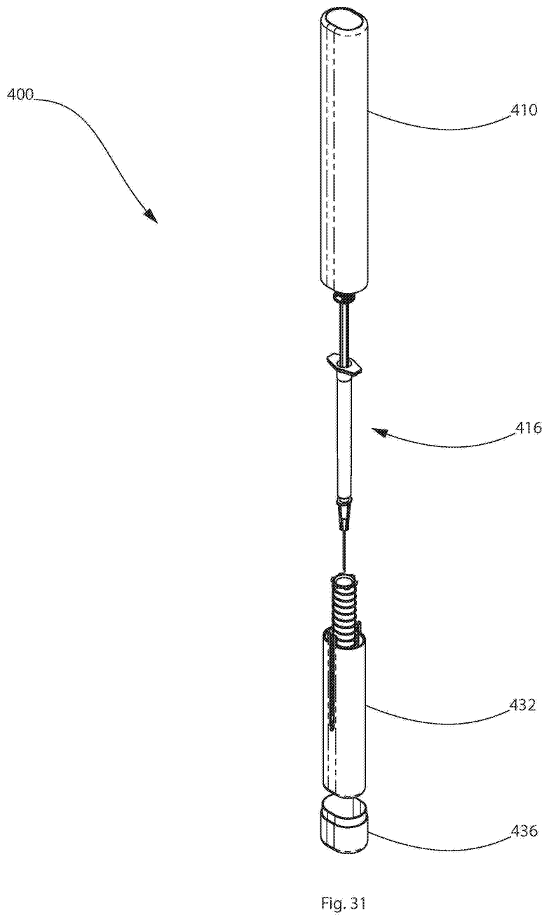

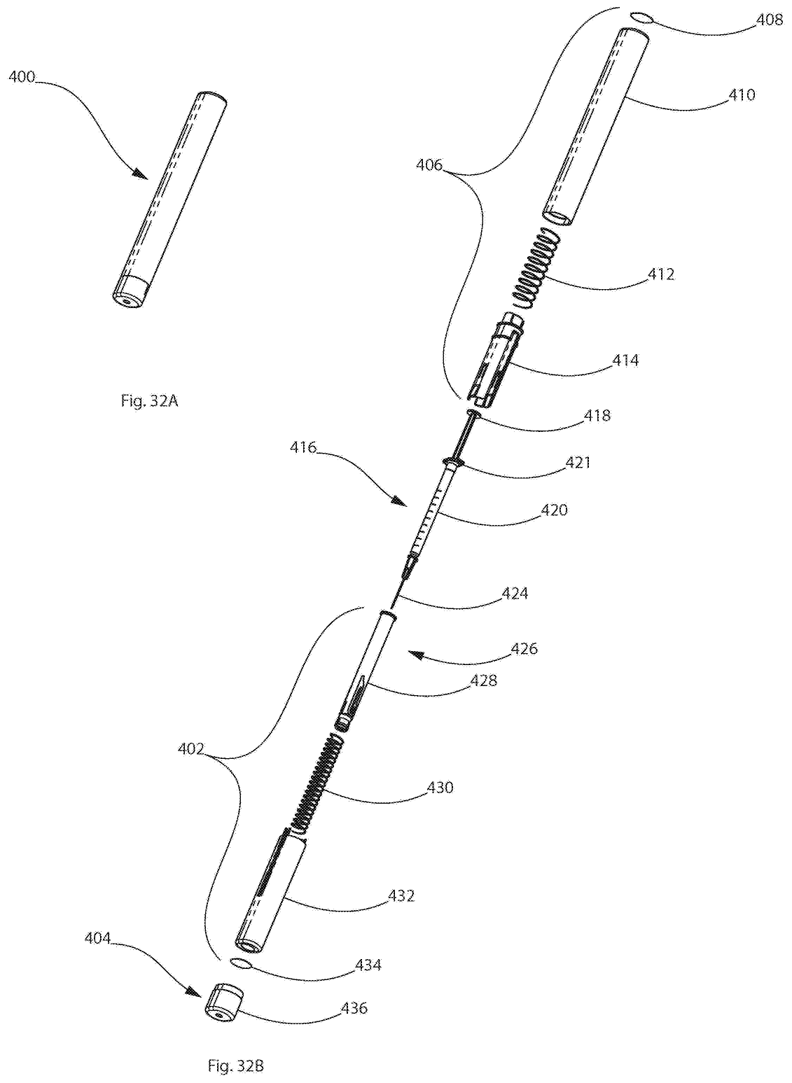

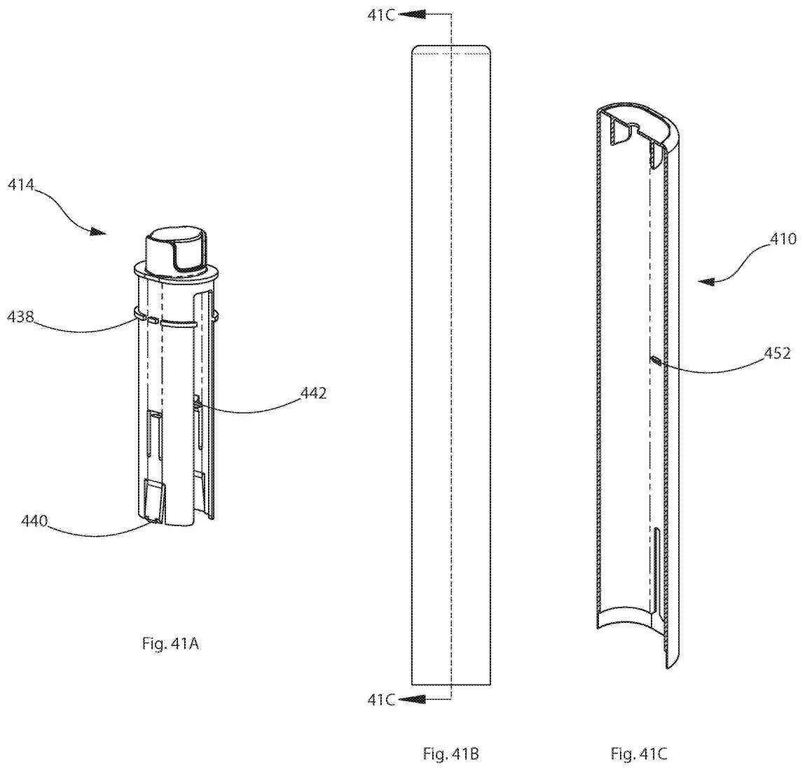

FIGS. 31-41 illustrate another embodiment of a device 400 for administering a shot from a standard pre-filled syringe 416. Such a syringe 416 may comprise a barrel 420, plunger 418 that slides within barrel 420, and needle 424. The barrel 420 typically has flanges 421 at one end that aid in pushing the plunger 418 through the barrel 420 containing medicament (not shown) to be administered by a user or patient.

Medicament device 400 preferably comprises a body assembly 402, a cap assembly 404 at one end of the body assembly 402, and a top assembly 406 at the other end of the body assembly 402 with its optional label 408.

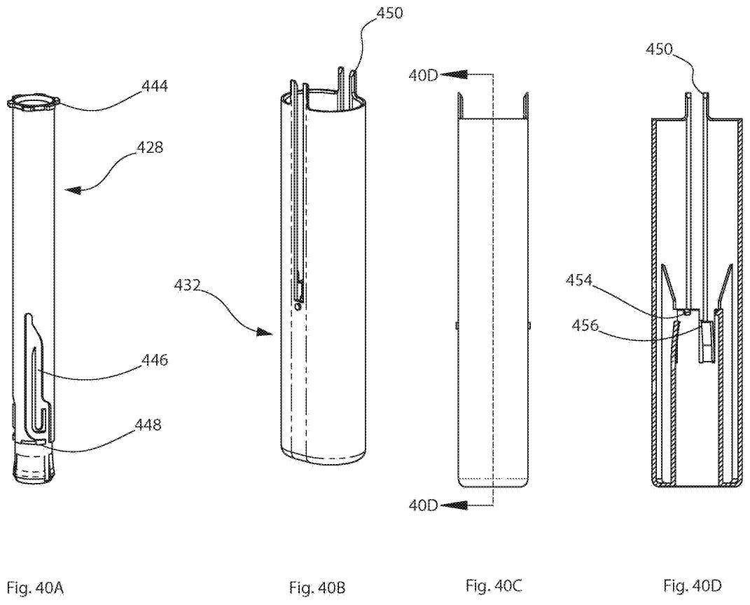

Body assembly 402 may be comprised of a sheath 432 with its optional sheath seal 434, locking mechanism 426, and sheath spring 430. The locking assembly 426 preferably comprises a sleeve 428 having a sleeve ledge 448, sheath catch 452, and sleeve tracks 446 configured to receive one or more sheath pins 454 protruding from the interior of sheath 432, as well as sheath snaps 456 disposed about the interior of sheath 432 and protruding therefrom, as illustrated in FIGS. 40A-40D. Sheath seal 434 may serve to seal the chamber of syringe 416 until pierced by the needle 424.

Cap assembly 404 may include cap 436. Top assembly 406 may include top 410, pusher spring 412, pusher 414 having pusher stops 438, pusher first snaps 440, pusher second snaps 442, and top label 408. Sheath spring 430 requires less compression force than the pusher spring 412.

As best seen in FIG. 31, top 410 is generally tubular in shape with a top and an opposing bottom. The top of top 410 is configured to receive and frictionally engage conventional syringe 416 placed into top 410 from the top. The top of top 410 is also configured to receive the sleeve 432 having an opening at one end configured to receive syringe 416. The bottom of top 410 is configured to receive the sheath 432 wherein the sheath 432 may freely slide within top 410.

The overall length of top 410 is about the same length of the barrel 420 of conventional syringe 416. Similarly, the overall length of sheath 432 with its sheath fingers 450 is also about the same length of the barrel 420.

When assembled, the entire syringe 416 with its needle 424 is encased within the device 400. Sheath cap 436 and sheath 432 surround the needle 416 when the device 400 is not in use.

Cap 436 then seals top 410.

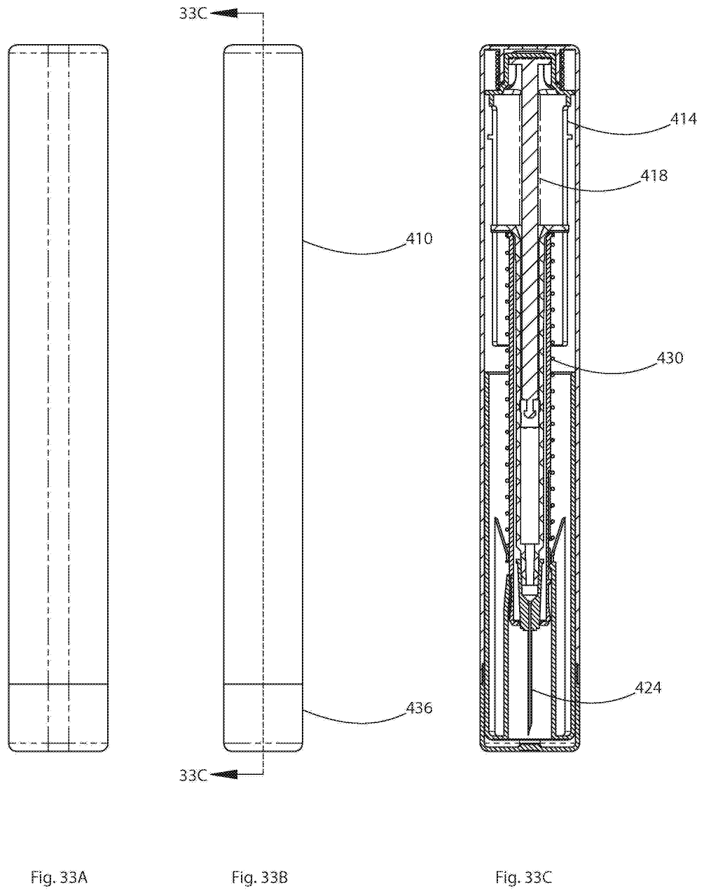

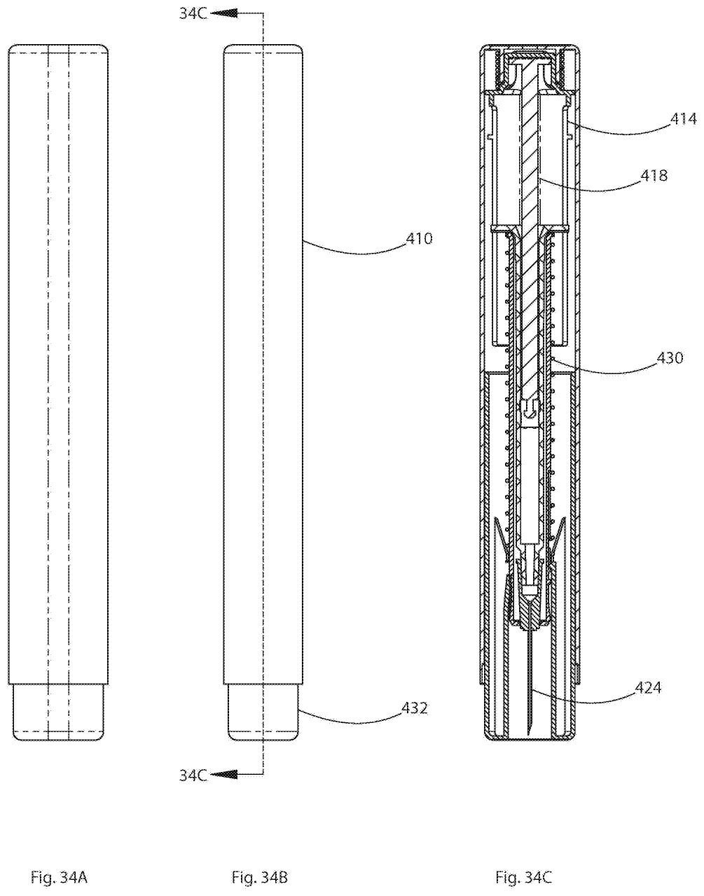

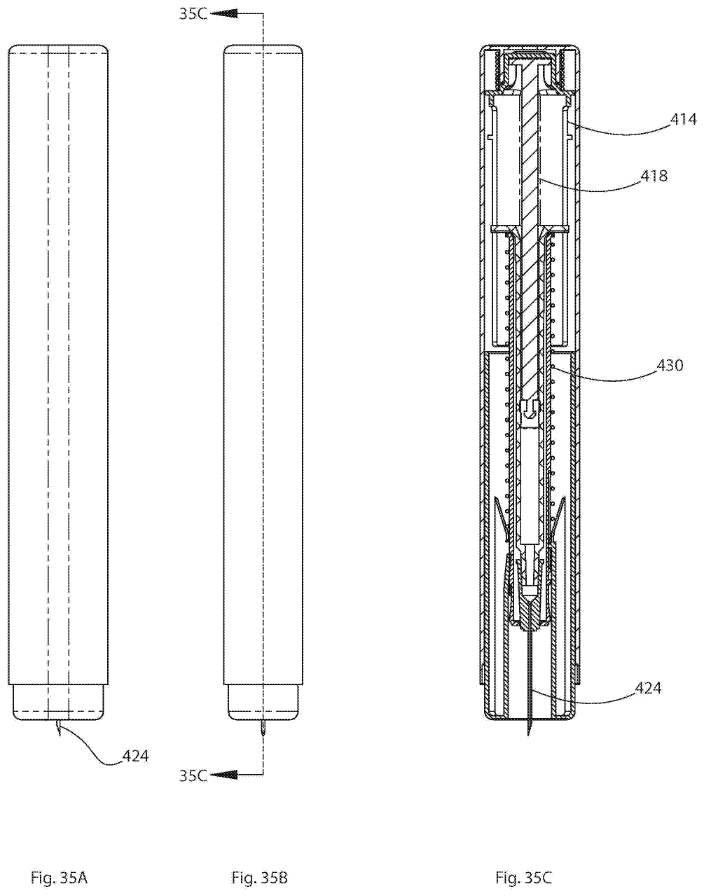

FIGS. 33A-39C show the medicament device 400 in various stages of use. FIGS. 33A-33C show the device 400 fully assembled and capped by cap 436 for safe storage and transportation. FIGS. 34A-34C show the device 400 with cap 436 removed. FIGS. 35A-35C show the device 400 in a stage whereby the needle 424 is deployed to deliver medicament.

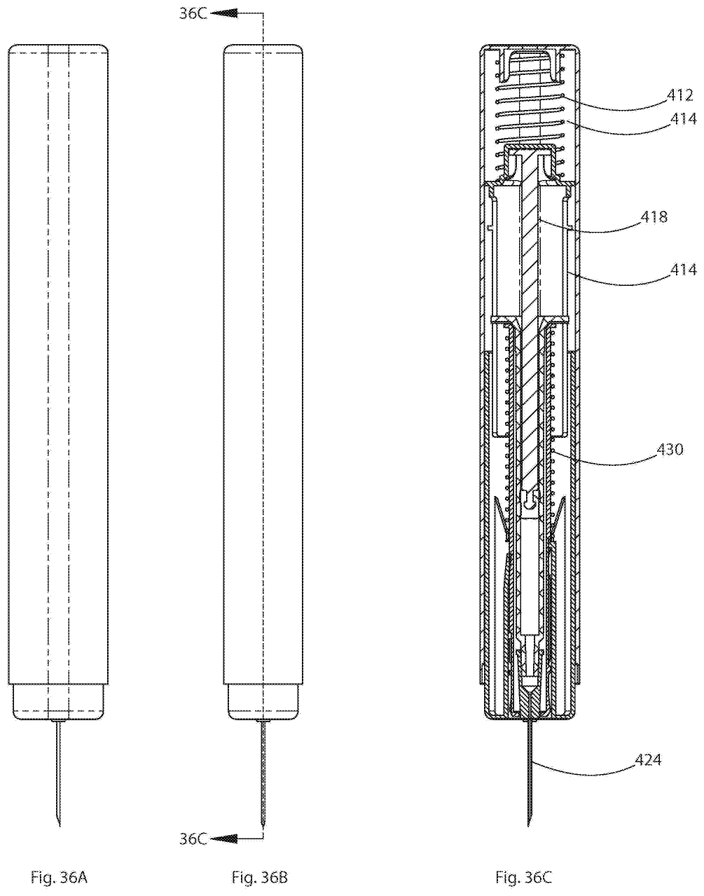

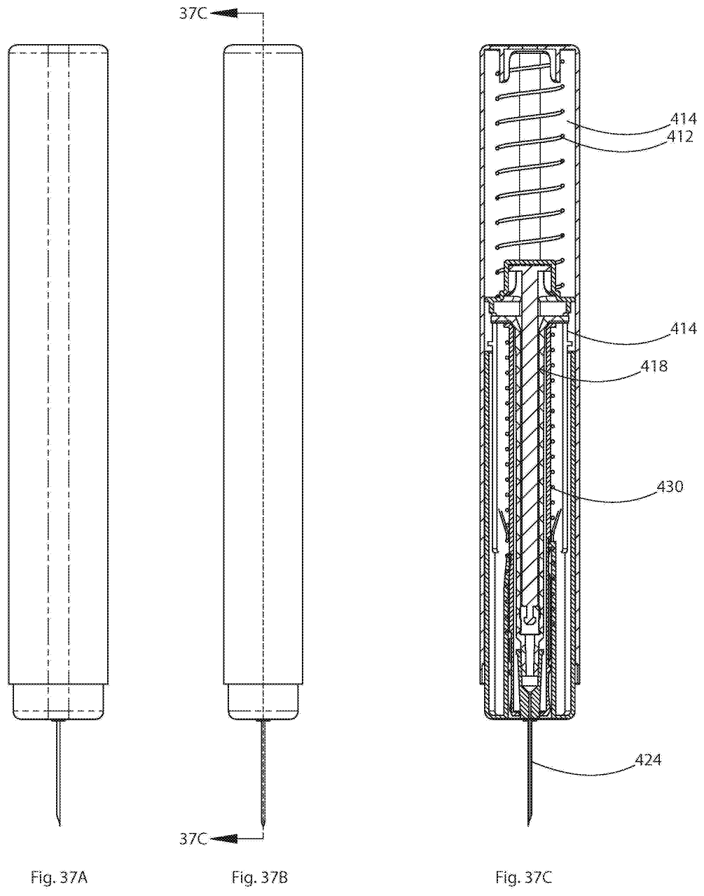

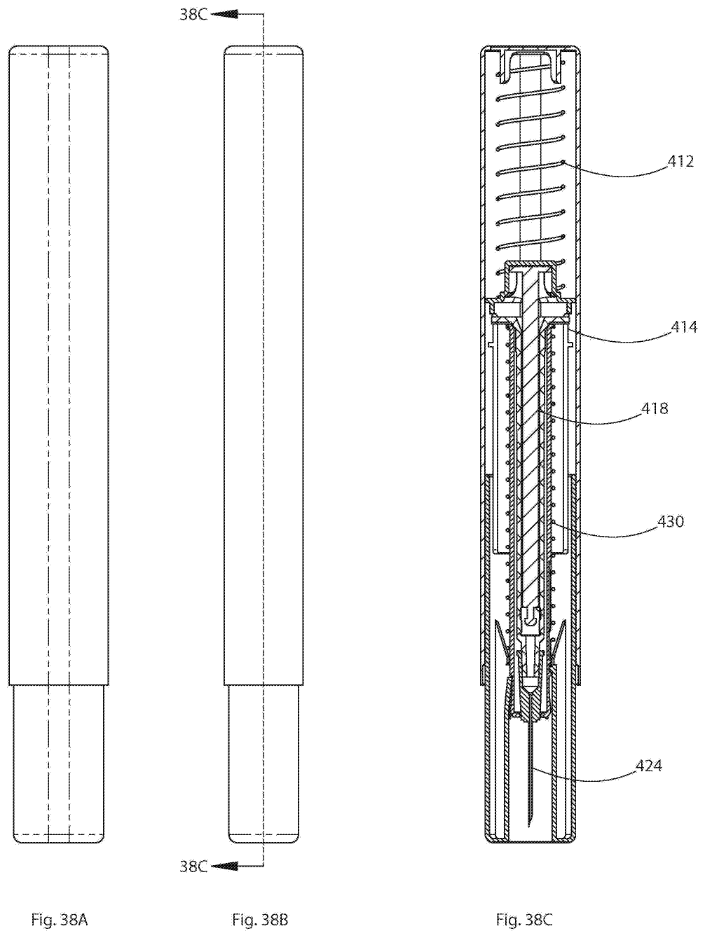

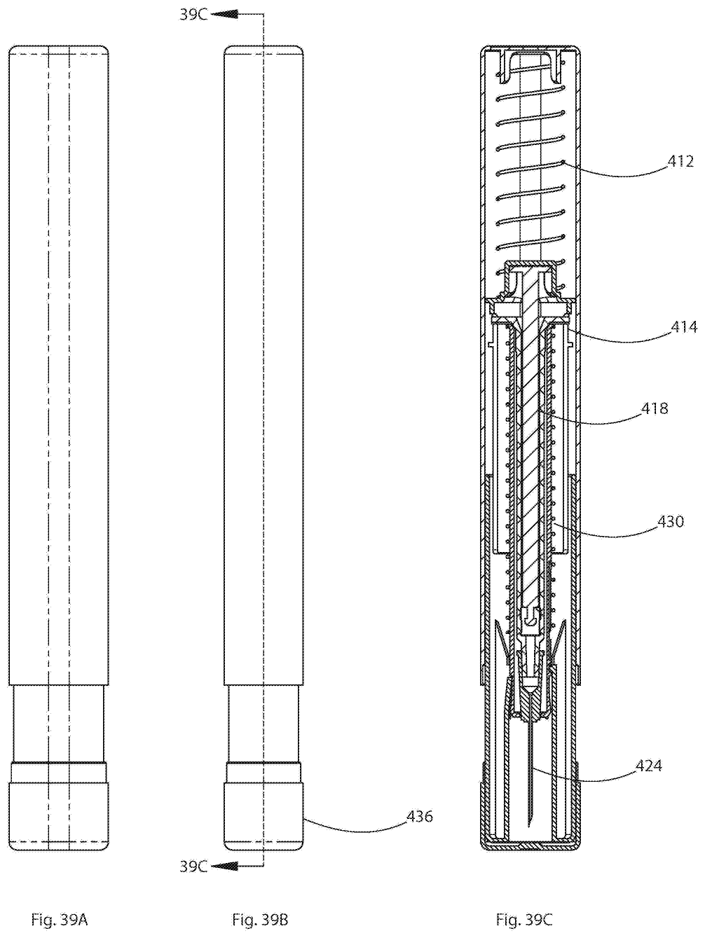

FIGS. 36A-36C show the device 400 with spring 430 in a compressed state. FIGS. 37A-37C show the device 400 with spring 412 in an uncompressed state wherein medicament has been delivered or is being delivered. FIGS. 38A-38C show the device 400 in a fully released state after the medicament has been delivered. FIGS. 39A-39C show the device 400 in a fully released and locked state with the device 400 being recapped for disposal.

In typical operation, there are three stages of use: start, insert, and plunge, wherein sheath fingers 450 push snaps 456 releasing pusher 414 that depresses syringe plunger 418 dispensing syringe 416 contents. The user begins by removing the cap assembly 404. The sheath 432 is then placed against an intended injection site (e.g., deltoid, triceps, quadriceps), with the user then pushing down on the device 400. Continued pushing causes spring-loaded sheath 432 to retract into top 410, triggering the syringe needle 424 until it is completely inserted into the injection site, fully compressing sheath 432 wherein sheath fingers 450 slide within top 410. Concurrently, sheath pins 454 found on the internal surface of sliding sheath 334 track in sleeve tracks 342, effectively guiding and rotating the sliding sleeve 432 concentrically as the sheath 432 draws in, guided by bifurcated sleeve tracks 447, as best seen in FIG. 40A.

When sheath 432 is fully compressed into top 410, sheath fingers 450 contact and spread snaps 456, releasing pusher 414 to slide within top 410 as driven by the compressed pusher spring 416. Pusher 414 then depresses syringe plunger 418 fully into the syringe barrel 420, expelling the medicament through syringe needle 424.

The shot having been administered, the device 400 is then removed from the injection site, withdrawing syringe needle 424 and allowing spring-loaded sheath 432 to re-extend from top 410 due to the biasing force of sheath spring 430. Sheath pins 454 track in the straight and slightly longer bifurcation found in the now-rotated sleeve tracks 446. At full extension, sheath 432 bottoms out at a position slightly more extended than its initial position, thus allowing sheath snaps 456 to engage and lock out sheath 432 for the purpose of protecting syringe needle 424, thereby preventing further use of the device 400.

The syringe 416 of the device 400 may be pre-loaded by the user for pre-filled, portable injections (e.g., insulin, epinephrine). The device 400 may be produced in varying lengths and sizes to accommodate different syringe sizes (e.g., 0.3 mL, 1 mL, 3 mL). The device 400 may also be adjusted to dispense varying doses of medicament by means of a coarsely threaded cap assembly 404, which may be adjusted to vary the travel length of syringe plunger 418, with indices showing the relative amount of medicament to be dispensed. The top 410 may also include a window to provide a direct view of the medication. Such a window may be circumscribed with a color that, when matched with the medicament color as viewed through said window, indicates the medicament has expired. In such a situation, the syringe 416 may advantageously be changed out for a new syringe with non-expired medicament.

The sheath 432 may be marked with color or graphics to let the user know the syringe needle 424 is fully inserted (triggering medicament injection). In the alternative, a battery-powered light emitting diode or the like may illuminate to indicate the syringe needle 424 is fully inserted. The sleeve 428 may also be marked with color or graphics visible through a window in the top 410 let the user know injection is complete.

The top 410 may be made of a transparent material that reveals the inner functioning of the device 400, including revealing the amount of medicament in syringe 416. The top 410 may alternatively be made of transparent or translucent material and wrapped with an opaque label in a manner that reveals the medicament level in syringe 416 through a label window. The top 410 may similarly incorporate instructional graphics.

The device 400 may include a peel-off-cover antiseptic pad for the purpose of cleaning the injection site. Such a pad may be adhered to the top or cap assemblies, 406, 404, for example.

The top assembly 406 may permanently lock when inserted into top 410, sealing in syringe 416 to assure single-use only. In the alternative, the top assembly 406 may be removable to change out syringe 416 as desired so the device 400 may be further used. If desired to limit device 400 reuse, the top assembly 406 may be removable by means of a special tool.

The cap 436 may be press-fit onto top 410 for removal by twisting off. Cap 436 may be press-fit onto top 410 with a cam feature to facilitate removal as it is twisted. The cap 436 may also include an attachment point for a lanyard. The cap 436 may also be integrated with a label that covers the whole device 400, with scoring marks to tear off the portion of the label that is acting as a cap. Similarly, the amount and/or type of medicament may be printed graphically on the proximal body tip.

Sheath 432 may lock upon extension after the needle 424 is withdrawn and then unlocked by a tool such as a pick, paper clip, or small screwdriver.

The device 400 may be made of disposable, single-use materials, or alternatively, made of reusable materials that could withstand repeated sterilization. Regardless of the materials used, device 400 may incorporate a peel tab or breakaway part that renders the device 400 non-functional after a single use, insuring single-use only.

The device 400 may also include a split thrust washer located between the syringe flanges 421 and the body cap 436 that deforms when squeezed to provide a tactile and audible click when the plunger 418 is fully-depressed, letting the user know that the stroke is complete, all medicament is dispensed, and the shot has been successfully administered.

The device 400 may integrate a voice-chip to provide audible instructions and status such as when a medicament has expired or is nearing the expiration date. Similarly, the device 400 may integrate a chemical or mechanical timer to indicate when a determined number of days have passed since syringe filling, indicating expiration of contents. Such a timer may be activated when top assembly 406 is closed to begin the countdown to medicament expiration.

The device 400 may include a shrink-wrap thermoplastic with graphics wherein the wrap is applied to seal and retain the protective cap 436 and then peeled away for use. Similarly, the top 410 may be sheathed in shrink-wrap thermoplastic with graphics showing instructional information, expiration date, custom branding, or the like.

The device 400 may include a flat surface on an otherwise cylindrical top 410 to keep the device 400 from rolling. Such a flat surface may be used to print indicia via a felt-tip marker or the like.

The device 400 may include a rotating collar on an otherwise cylindrical top 410 that is positioned by the assembler or user to show the loaded medicament dose. The rotating collar may then be locked into place by virtue of device assembly.

The device 400 may include a metal sleeve 428 within top 410, which when pre-chilled with ice or refrigeration, provides cooling for heat-sensitive medicament. In the alternative or in addition thereto, the device 400 may be shaped to fit into a refrigerated chiller to pre-cool heat-sensitive medicament during transportation.

The top 410 may be configured to contain a plurality of syringes in the event that administration of initial injection is inadequate to treat the present condition (e.g., epinephrine administration for anaphylaxis typically calls for a second dose if symptoms do not abate in a timely manner) or for convenience of having more than one injection at hand (e.g., fertility injections are typically timed hourly).

The device 400 may also be used for oral medication dispensation, concealing a standard syringe and its association with needles.

The device 400 may incorporate a microchip, such as a radio-frequency identification or near-field communication chip, for geolocation, product information, expiration information, or other such status of the device 400 and its contents.

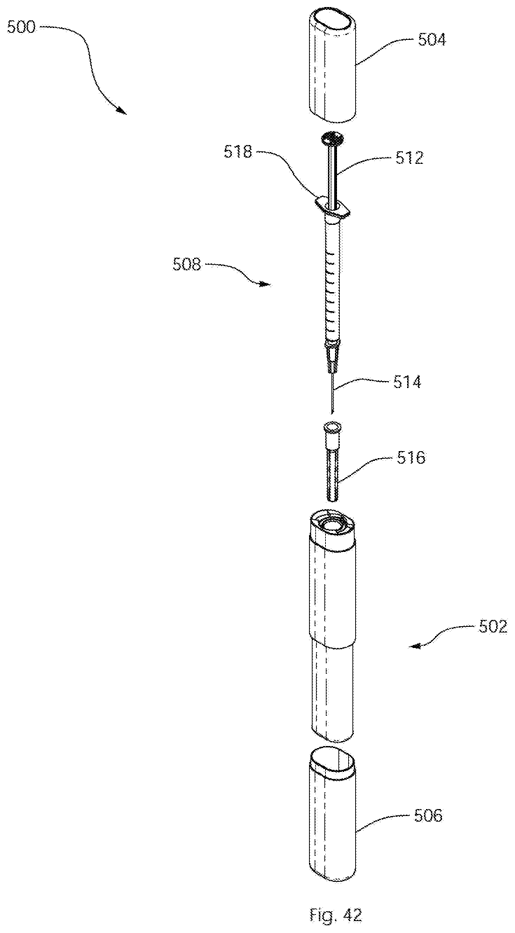

FIGS. 42-44C illustrate other embodiments of a medicament device 500 for administering a shot from a standard pre-filled syringe 508. Such a syringe 508 may comprise a barrel 510, plunger 512 that slides within barrel 510, needle 514, and its needle cover 516. The barrel 510 typically has flanges 518 at one end that aid in pushing the plunger 512 through the barrel 510 containing medicament (not shown) to be administered by a user or patient.

Medicament device 500 comprises a body assembly 502, a housing cap 504 at one end of the body assembly 502, and a sheath cap 506 at the other end of the body assembly 502.

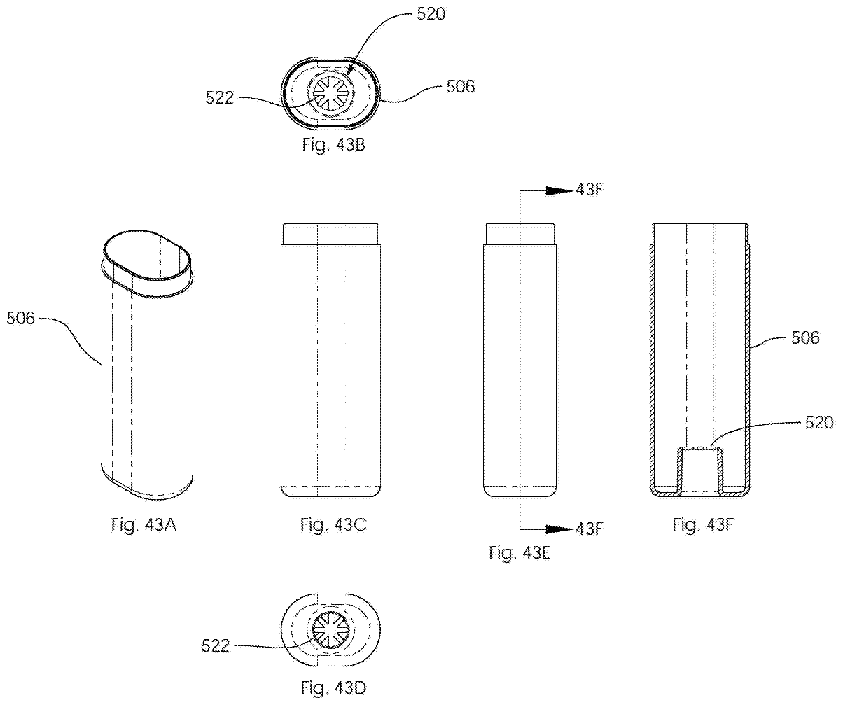

Medicament device 500 may include any aspects of the above-disclosed inventions 100, 200, 300, or 400, except for modifications to the sheath cap 506 and/or housing cap 504. As best seen in FIGS. 43A-43D, sheath cap 506 includes a cover stripper 520 disposed about one end of the sheath cap 506. Cover stripper 520 may be a ring with annular flexible stripper barbs 522 extending toward the center of the cover stripper 520. Cover stripper 520 may be integrated with sheath cap 506 creating a single, injection-moldable part. In the alternative, cover stripper 520 may be a separate molded or stamped plastic or metal ring, retained by a boss feature or the like within sheath cap 506.

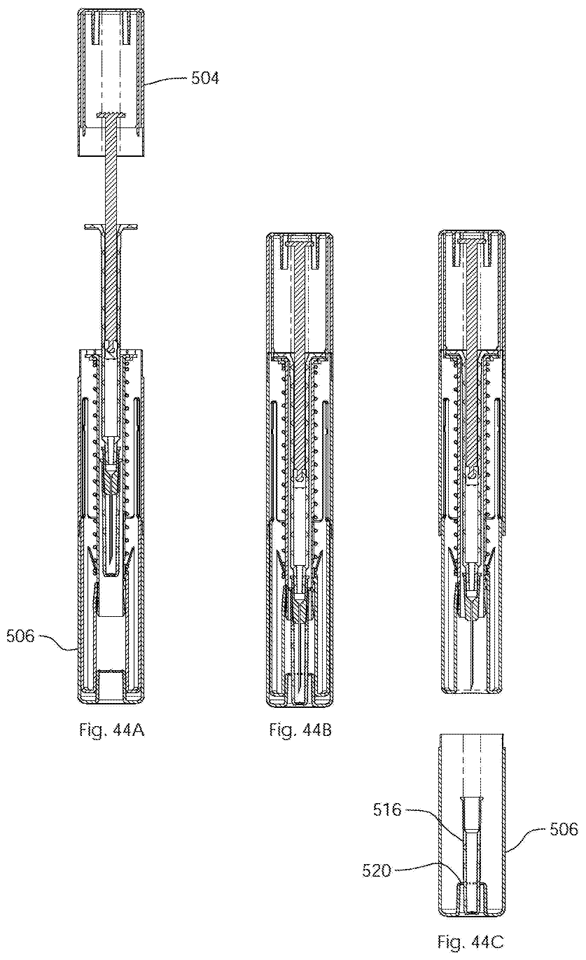

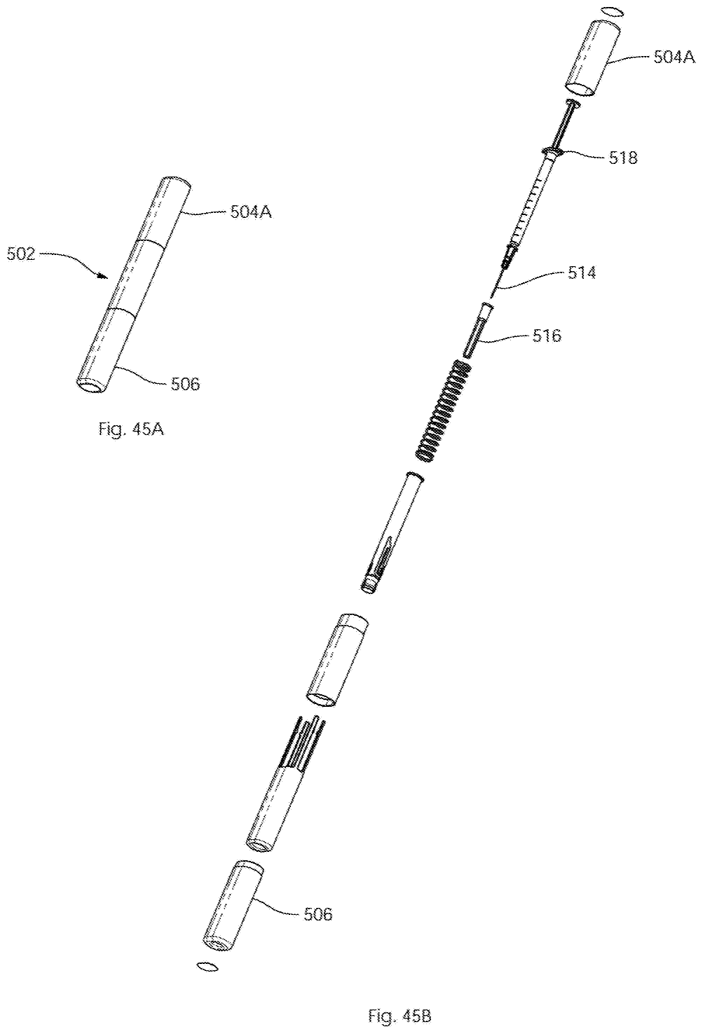

The operation of medicament device 500 may be seen with particular reference to FIGS. 44A-44C. Syringe 508 may be loaded into body assembly 502 in either a manual or automated fashion. As syringe 508 is loaded into body assembly 502, the tip of sheath cap 506 presses into and through cover stripper 520, resulting in cover stripper 520 circumscribing and capturing needle cover 516. Cover stripper 520 subsequently retains needle cover 516 as sheath cap 506 is removed for use of device 500, retaining needle cover 516 within sheath cap 506. After use of the device 500, body assembly 502 is recapped with sheath cap 506, effectively re-covering needle 508 for safe disposal of device 500.

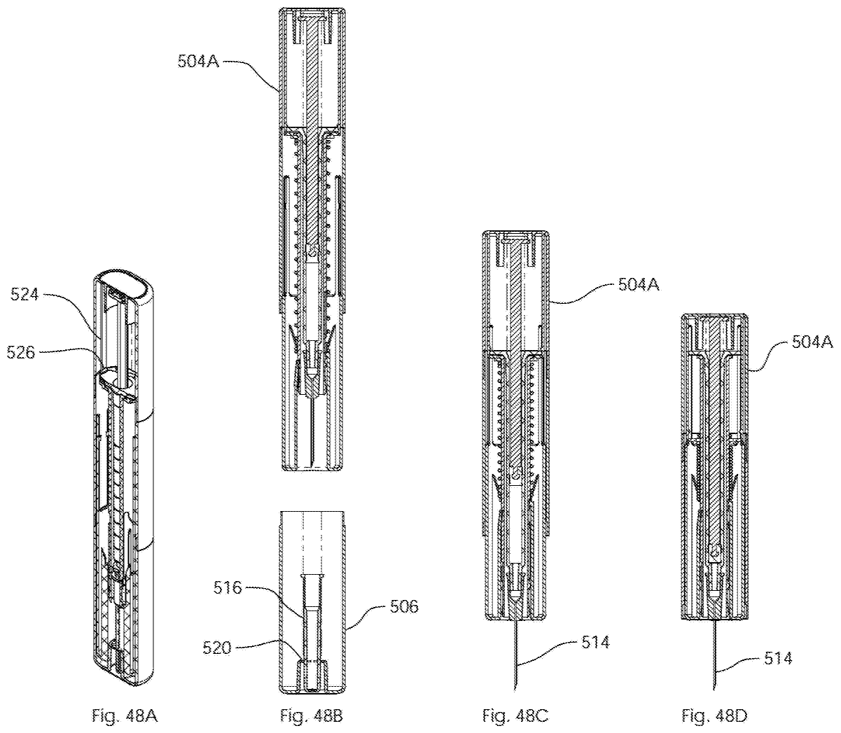

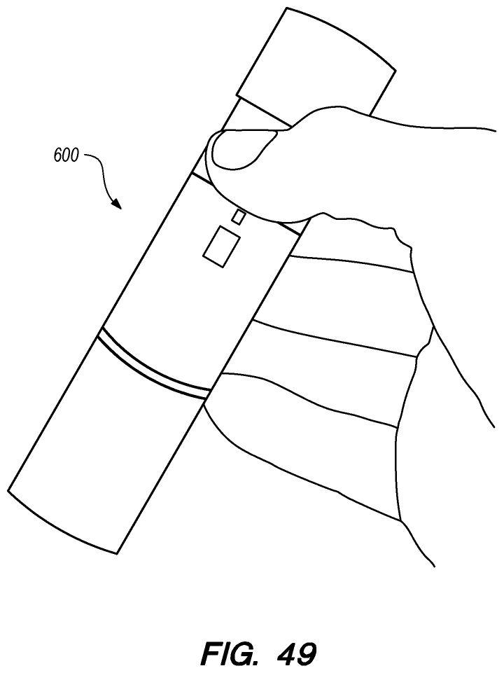

FIGS. 45A-48D illustrate another embodiment of a medicament device for administering a shot from a standard pre-filled syringe with needle 514, needle cover 516, and flanges 518. The medicament device may include any aspects of the above-disclosed inventions 100, 200, 300, 400, 500 except for the additional modification to the housing cap 504A to ensure positive syringe seating.

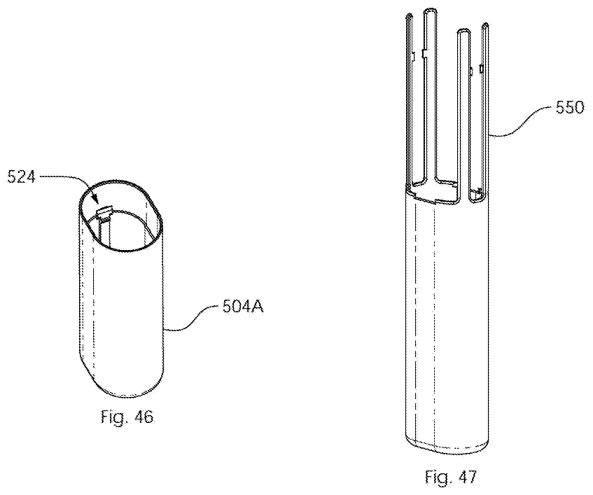

FIGS. 46A-46B particularly show a modification to housing cap 504A that may be used with cover stripper 520 as disclosed above. As seen in FIGS. 46-48D, positive syringe seating is assured automatically during manual or automated housing cap 504A placement by means of dual opposing seating legs 524 within housing cap 504A, positioned such that legs 524 push against the syringe flanges 518 as housing cap 504A is pressed onto a body assembly 502. Doing so pushes needle cover 516 through cover stripper 520, deforming its barbs to retain needle cover 516 in sheath 506. As seating legs 524 contact syringe flanges 518 and fully seat syringe within the body assembly 502, ramps 526 on seating legs 524 guide the sheath legs 550 outward and away from flanges 518. As a result, the syringe will rise within the body assembly 502 to complete the injection stroke of the medicament device.

These modifications advantageously allow a conventional syringe with sterile needle 514 to remain covered during loading into a body assembly 502, avoiding danger of user needle stick, or needle contamination such as the user contacting the exposed needle 514, or the exposed needle 514 touching an inner wall of a body assembly 502 as it is being loaded.

With needle cover 516 intact throughout loading, the medicament device need not be sterilized, as needle 514 remains covered during loading into a body assembly, and throughout storage and transport of the medicament device. When the device is used, sheath cap 506 is manually removed by user, stripping needle cover 516 with it, exposing and readying needle 514, yet needle 514 remains shielded from user inside sheath cap 506.

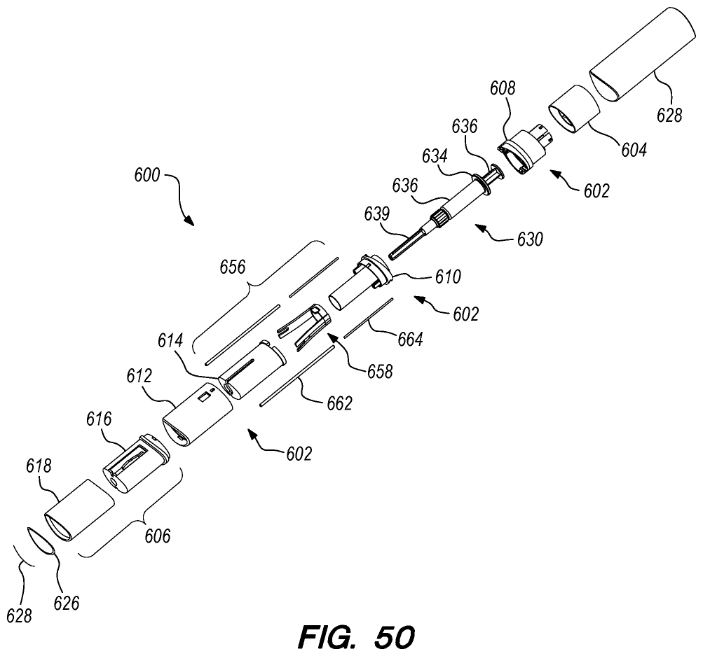

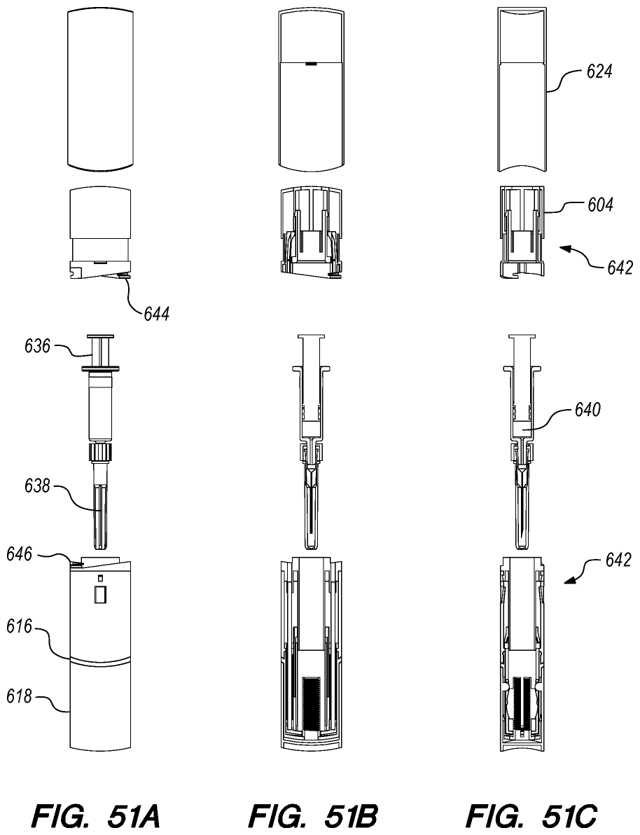

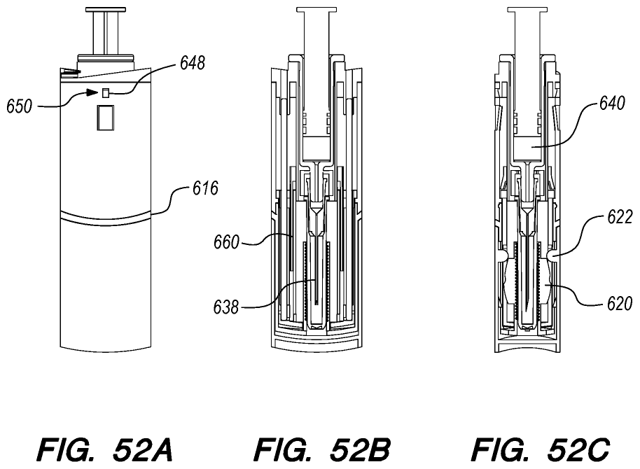

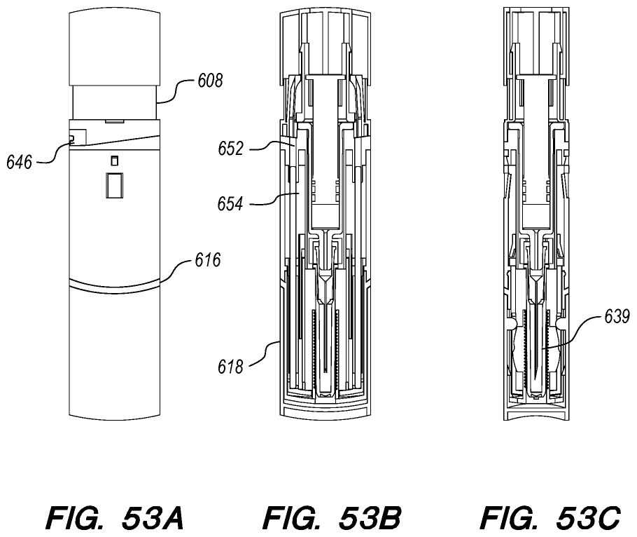

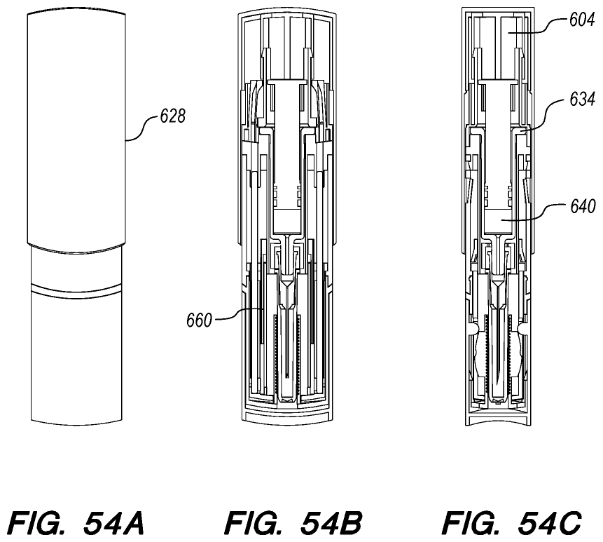

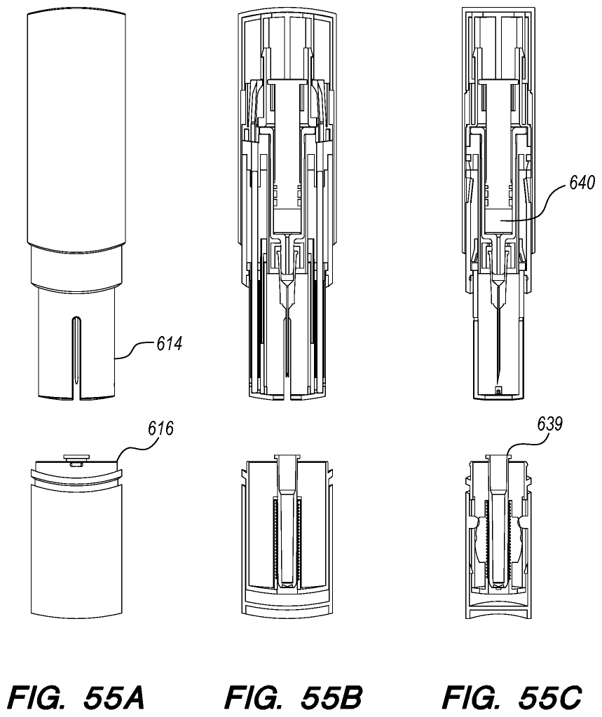

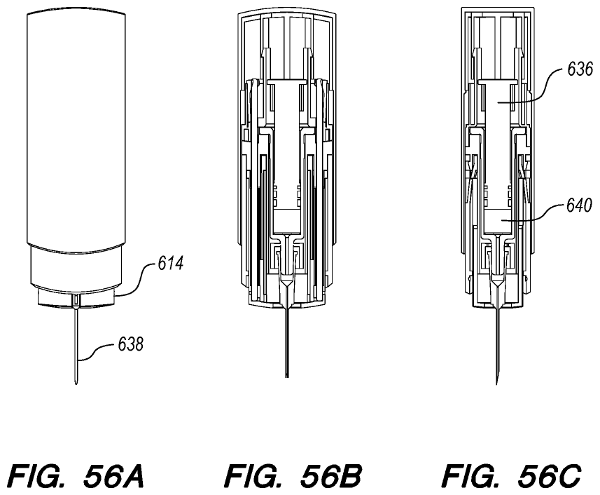

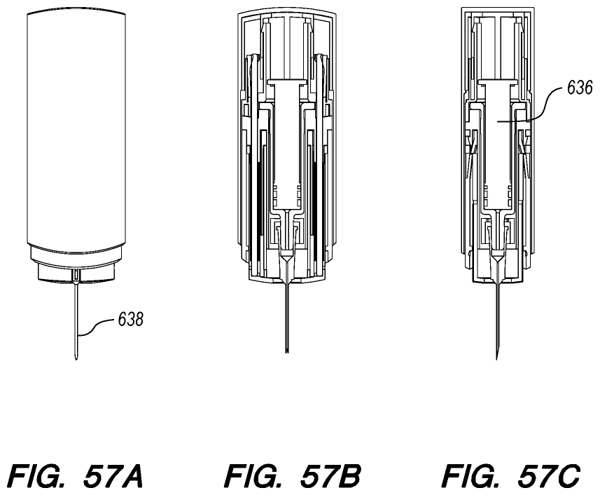

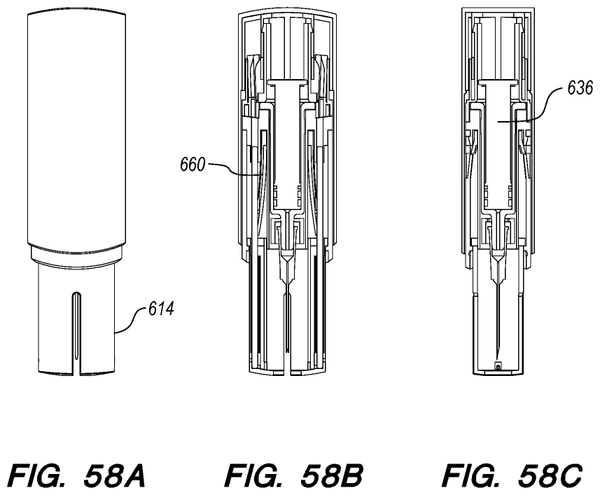

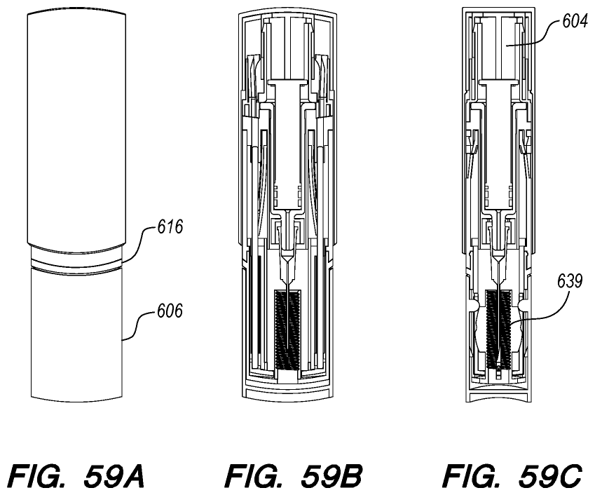

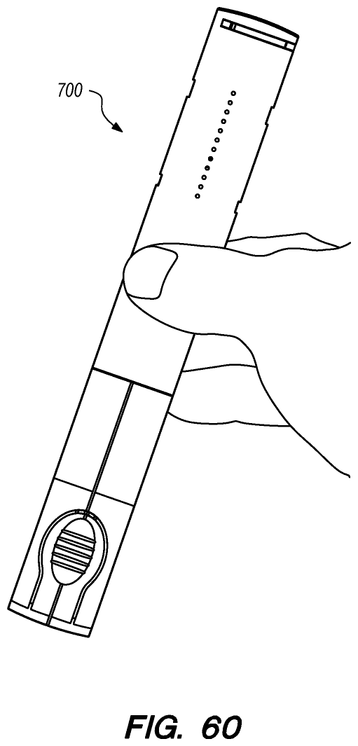

FIGS. 49-59C illustrate another embodiment of a medicament device 600 for administering a shot from a standard pre-filled syringe 630 such as 1 ml Becton Dickinson tuberculin syringe filled with any desired medication 640 such as a vaccine for COVID-19. Device 600 may include any aspects of the above-disclosed inventions except as noted herein.

Syringe 630 may comprise barrel 632, plunger 634 that slides within barrel 632, needle 638, and its needle cover 639. The barrel 632 has flanges 634 at one end. The plunger 636 moves within barrel 632 to dispense medication 640. Depending on the intended use of device 600, medication 640 may be injected into a site (not shown) such as a deltoid, triceps, quadriceps or the like of a user or patient.

Medicament device 600 preferably comprises a body assembly 602 having a top and an opposing bottom end, an inner cap 604 disposed about the top of the assembly 602, and a sheath cap 606 disposed about the bottom of assembly 602. Body assembly 102 may be comprised of upper housing 608, inner housing 610, and lower housing 612 containing sheath 614. The inner housing 610 snaps into lower housing 612 via a housing lock detent 648 protruding from the inner housing and housing lock channel 650 cut into lower housing 612. The inner cap 604 slides over and is retained by the upper housing 608 to actuate the syringe plunger 636 for dispensing medication 640. An optional outer cap 624 may be disposed about the inner cap 604. The outer cap 624 provides the user with a solid gripping surface and may contain labels for proper instruction and handling of device 600.

Sheath cap 606 preferably includes a needle cover stripper 616 and a stripper cap 618 receiving needle cover stripper 616. The needle cover stripper 616 is adapted to seat within lower housing 612, wherein flanges extending from the cover stripper 616 limit insertion of the cover stripper 616 within lower housing 612. A plurality of ramps 620 on the interior surface of stripper cap 618 are adapted to engage with respective flexible fingers 622 in cover stripper 616.

An optional wipe pad 626 may be disposed about one end of the stripper cap 618. The wipe pad 626 may comprise an antiseptic-impregnated gauze for the recommended cleaning of the injection site pre and post injection. An optional foil seal 628 may be adhered to the stripper cap 618 to cover the wipe pad 626.

A plunger lock mechanism 642 retains the syringe 630 in the body assembly 602. The plunger lock mechanism 642 preferably comprises a bayonet lock arrangement comprising bayonet lock arms 644 extending from the upper housing 608 and a respective bayonet lock channel 646 cut into the inner housing 610 for receiving the arms 644. Other means of locking the plunger 636 may be utilized such as a track and detent arrangement or the like.

A sheath lock mechanism 656 serves to lock the sheath 614 in place after injection. Sheath lock mechanism 656 preferably comprises sheath lock 658, guide rods 662, and springs 664. Sheath lock 658 includes legs 660 extending downward from the top of its body. The legs 660 are initially constrained within the sheath 614. When unconstrained upon completion of the injection stroke, the legs 660 expand outward to lock the sheath 614 in the extended position. The guide rods 662 are anchored on either side of the sheath 614 within blind holes 654 cut therein. The inner housing 610 has guide rod tunnels 652 with each tunnel 652 adapted to receive one end of a respective guide rod 662. The rods 662 move with the sheath 614 as it retracts, thus disengaging the inner cap 604 and upper housing 608 at a point only after the needle 638 is fully inserted. In this way, syringe plunger 636 can only depress after the sheath 614 is retracted and the needle 638 fully inserted. The springs 664 encircle the guide rods 662. Springs 664 are compressed as sheath 614 retracts into housing 612 upon needle 638 insertion, creating potential energy to return sheath 614 to the extended position upon stroke completion.

In operation, a filled syringe 630 is dropped into upper housing 608, its flanges 634 bottoming out of the inner housing 610. The needle cover 639 remains intact to seal the sterile needle 638 throughout loading, maintaining its sterility. With the plunger lock mechanism 642 as illustrated, once the syringe 630 is dropped in, the upper housing 608 is rotated, causing lock arms 644 to slide within channel 646 to maintain plunger 636 in place and unable to move within barrel 632.

The foil seal 628 is then peeled off and the wipe pad 626 utilized as desired.

The user then pulls off the sheath cap 606. Doing so causes the needle cover stripper 616 to grab and remove the needle cover 639 as the sheath cap 606 is removed. In particular, the plurality of ramps 620 on the interior surface of stripper cap 618 press to deform respective flexible fingers 622 in the cover stripper 616, effectively tightening upon the needle cover 639, actuated by the action of the user pulling off the sheath cap 606. The needle cover 639 is retained with the sheath cap 606. The sheath cap 606 may be returned to re-cover the sheath 614 after injection for safe disposal of device 600.

The user then depresses sheath 614 against the injection site. The sheath 614 retracts progressively against the springs 664. Doing so reveals once-concealed needle 638. As the needle 638 is inserted into the injection site, the syringe 630 is stabilized by guide rods 662 contained in blind holes 654 running the length of the sheath 614. The sheath 614 is limited in travel by flanges on the housing to limit its extension. Only when the needle 638 is fully inserted into the injection site does the sheath 614 disengage the inner cap 604 and upper housing 608, thus compressing plunger 636 to dispense medication 640.

Once fully dispensed, the user stops pushing the device 600 against the injection site, causing the springs 664 to uncompress. The sheath 614 then returns to its fully extended position, wherein the legs 660 of the sheath lock 658 now abut and are locked against the top of the sheath 614. In this manner, the sheath lock mechanism 656 serves to keep the needle safely and firmly concealed within sheath 614 after use of the device 600.

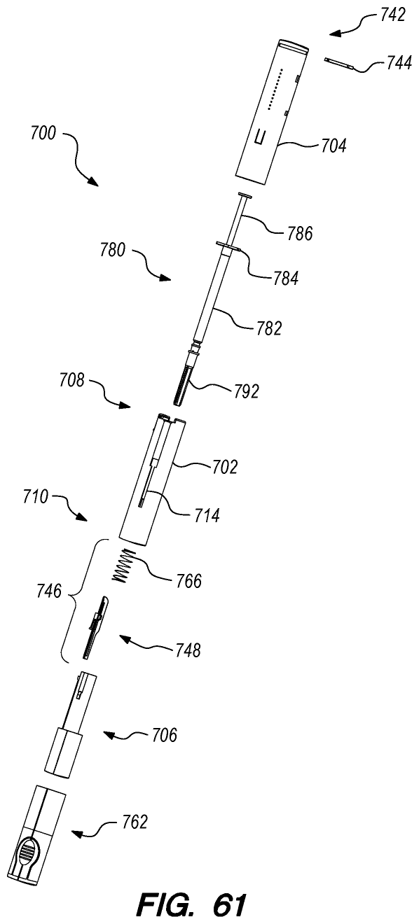

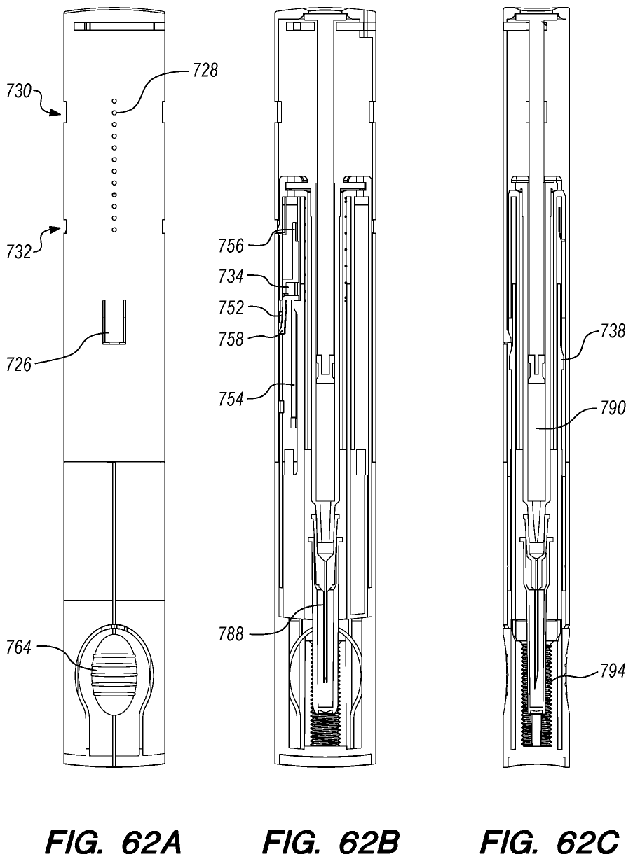

FIGS. 60-71C illustrate another embodiment of a medicament device 700 for administering a shot from a standard pre-filled syringe 780 such as 1 ml Becton Dickinson tuberculin syringe filled with medication 790 such as a vaccine for COVID-19. The medicament device 700 may include any aspects of the above-disclosed inventions except where noted.

Syringe 780 may comprise barrel 782, plunger 786 that slides within barrel 782, needle 788, and its needle cover 792. The barrel 782 has flanges 784 at one end. The plunger 786 moves within barrel 782 to dispense medication 790. Depending on the intended use of device 700, medication 790 may be injected into a site (not shown) such as a deltoid, triceps, quadriceps or the like of a user or patient.

Medicament device 700 preferably comprises a housing 702 having a top end 708 and an opposing bottom end 710, a top cap 704 disposed about the top end 708, and a sheath 706 disposed about the bottom end 710. Housing 702 includes retaining slots 712 at the top end 708 for retaining the flanges 784 of syringe 780. A track 714 runs along the body of housing 702. A locking tab 716 extends away from the body of housing 702 near the top end 708. Housing 702 also has an upper hole 718, lower hole 720, and stop hole 722, each cut therewithin.

The top cap 704 includes a lock slot 724 disposed within its top end, and a sprung tab 726 extending away from near a mid-point of the top cap 704. A window 728, which is comprised of holes, is cut into top cap 704. The top cap 704 also has an upper hole 730 and a lower hole 732, each cut therewithin.

The sheath 706 comprises a body 734 with legs 736 extending upward therefrom. Ramps 738 protrude from the sides of legs 736. A ledge 740 extends outward from one of the legs 736. The device 700 preferably includes a sheath lock mechanism 746. The sheath lock mechanism 746 comprises a sheath lock 748 and spring 706. The spring 766 is disposed between the housing 702 and sheath 706. The sheath lock 748 includes a body 750, a first leg 752 extending downward from the body 750, a second leg 754 extending downward from the body 750 substantially parallel to the first leg 752, and a third leg 756 extending upward from the body 750. The first leg 752 includes catch 758 disposed about an end furthest away from body 750. The second leg 756 includes sheath stop 760 disposed about an end furthest away from the body 750.

A sheath cap 762 is disposed about sheath 706. The sheath cap 762 includes finger pads 764 cut therewithin and grippers 794 in the interior thereof.

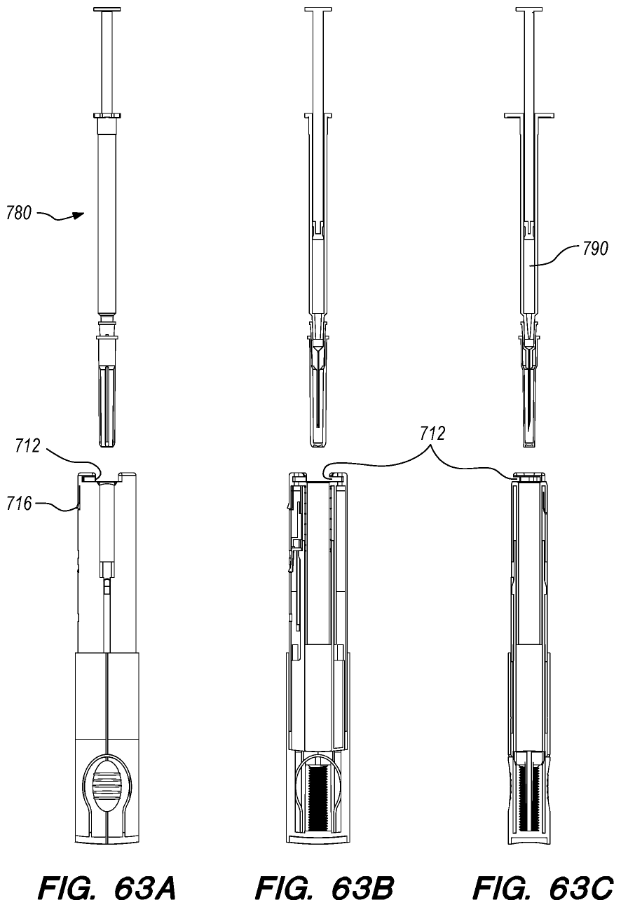

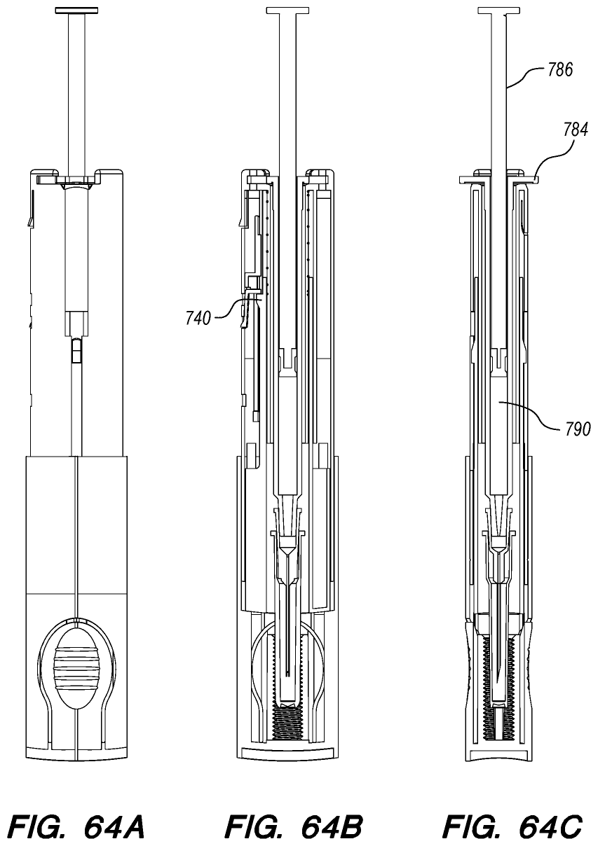

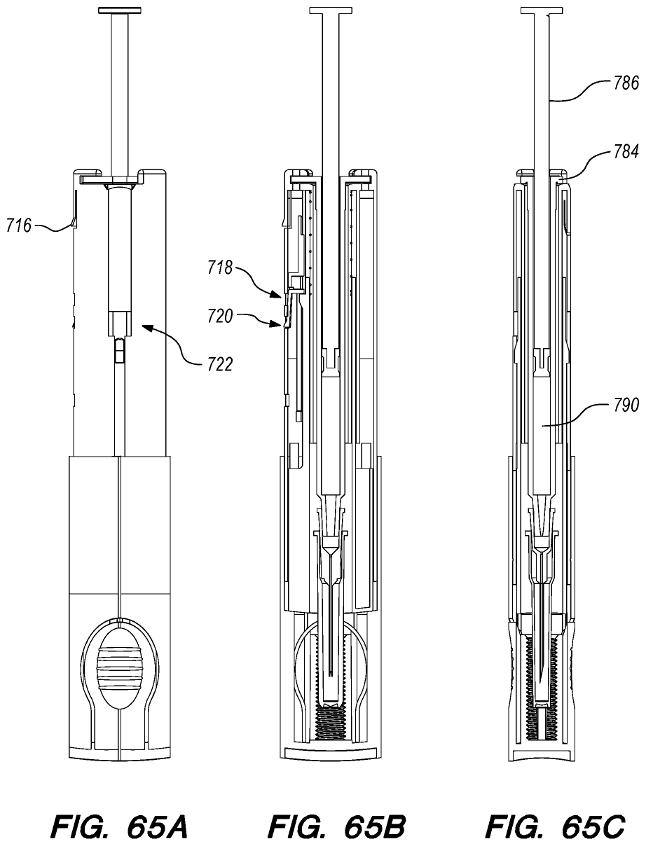

In operation, the user holds device 700 in their hand as seen in FIG. 60. The device 700 in its fully assembled state may come already loaded with syringe 780 and applicable medication 790. Should the device 700 not come with a pre-loaded syringe 780, the user may pull off top cap 704 to ready the device for a syringe 780 with medication 790 to be inserted within housing 702, as illustrated in FIG. 63A. Next, the user drops the filled syringe 780 into the housing 702, with the flanges 784 bottoming out against the top end 708 as seen in FIG. 64A. The user then twists the syringe 780 wherein its flanges 784 seat with retaining slots 712 to keep the syringe 780 in place as seen in FIG. 65A. In this state, the sheath lock mechanism 746 has its catch 758 seated within lower hole 720 of housing 702.

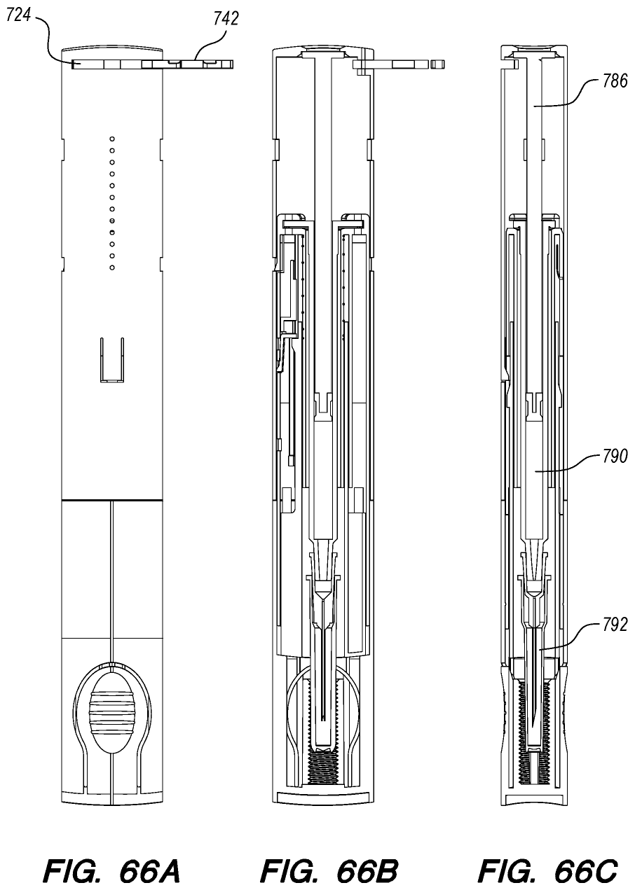

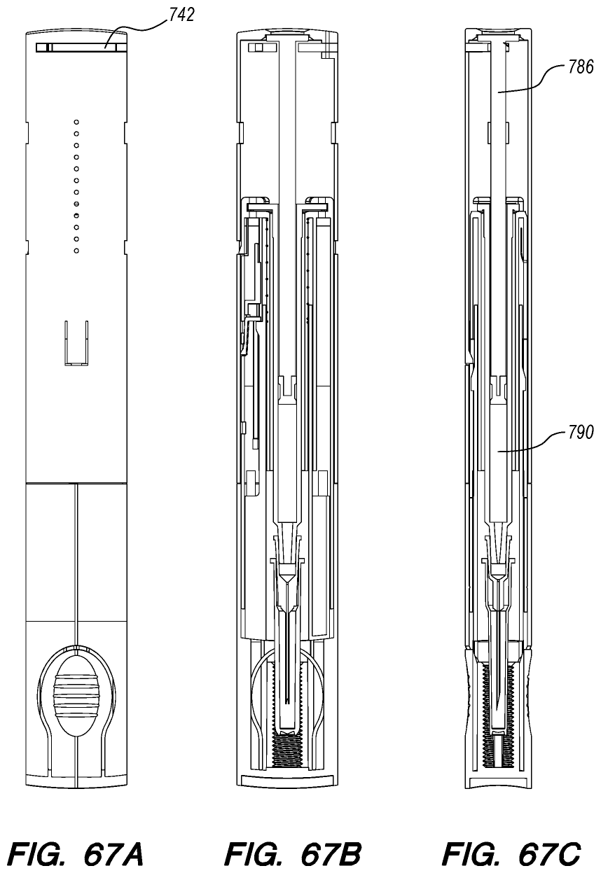

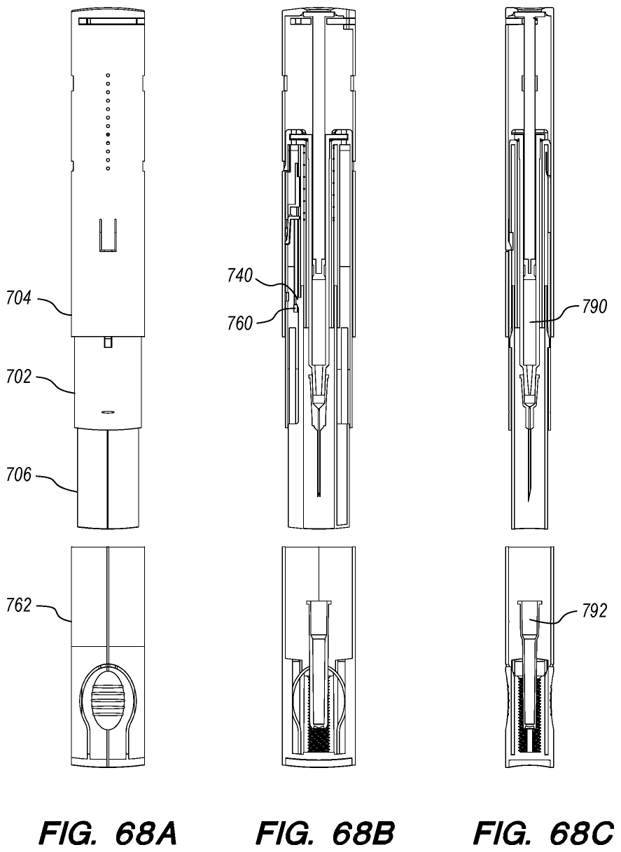

Next, the user then places the top cap 704 onto housing 702 until sprung tab 726 engages stop hole 722 of housing 702 to keep the cap 704 snug around the housing 702 as seen in FIG. 66A. The user then begins to engage the plunger lock mechanism 742 wherein plunger lock 744 is partially fitted within lock slot 724 of top cap 704. Next, the user fully engages the plunger lock mechanism 742 by completely fitting plunger lock 744 within lock slot 724 to maintain the plunger 786 in place as seen in FIG. 67A. The user then pulls downward on the sheath cap 762 and preferably does so by grabbing the finger pads 764 cut within the sheath cap 762. Doing so causes grippers 794 in the interior of sheath cap 762 to grab onto the cover 792 of the syringe 780. Further pulling removes the sheath cap 762 from the device 700 wherein cover 792 is now fully contained within sheath cap 762 as illustrated in FIG. 68A.

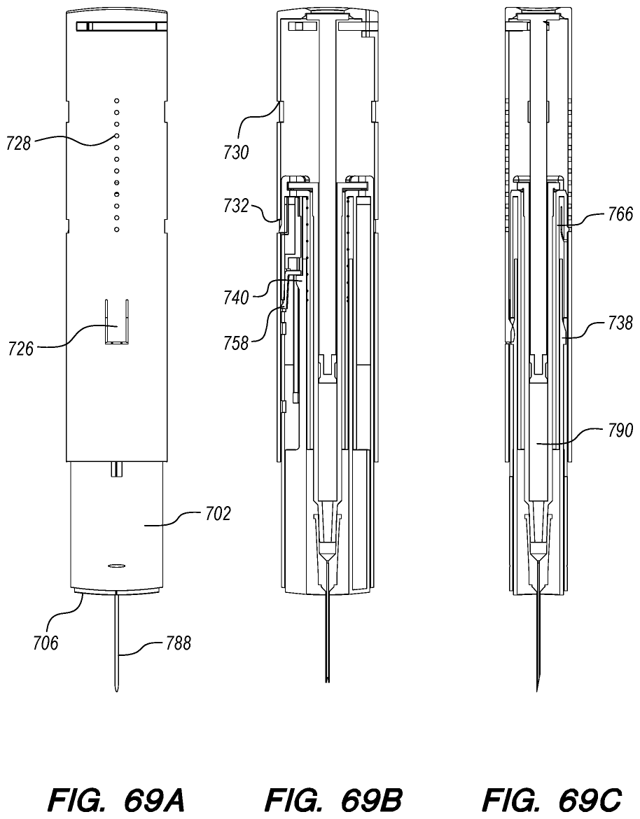

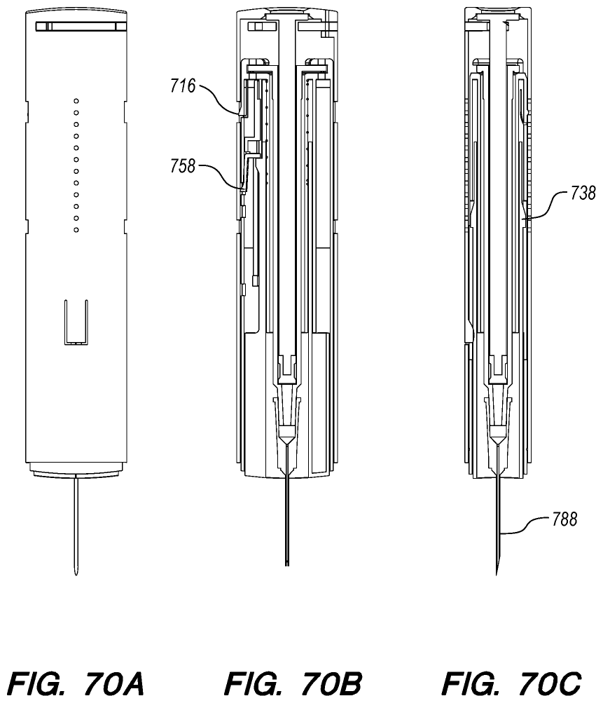

The user then begins to administer an injection against an injection site. To do so, the sheath 706 is pushed against the injection site, forcing the sheath 706 to move upward into housing 702 as seen in FIG. 69A. At this state, the ramps 738 of sheath 706 are contacting spring tab 726 of top cap 704. Further pushing against the injection site causes full deflection of sprung tab 726 wherein the housing 702 is now permitted to slide up within top cap 704, spring 766 is fully compressed, and catch 758 moves from lower hole 720 to upper hole 718 of housing 702 via upward sliding of sheath lock 748 in track 714 of housing 702. Doing so results in the ledge 740 of the sheath 706 to abut against the body 750 of sheath lock 748 prohibiting further movement of sheath 706 within housing 702. The plunger 786 is thus allowed to be pushed downward within barrel 782 to dispense medication 790 contained within syringe 780 as seen in FIG. 70A. The user may see the sliding of the sheath 706 into housing 702 via window 728 cut within top cap 704.

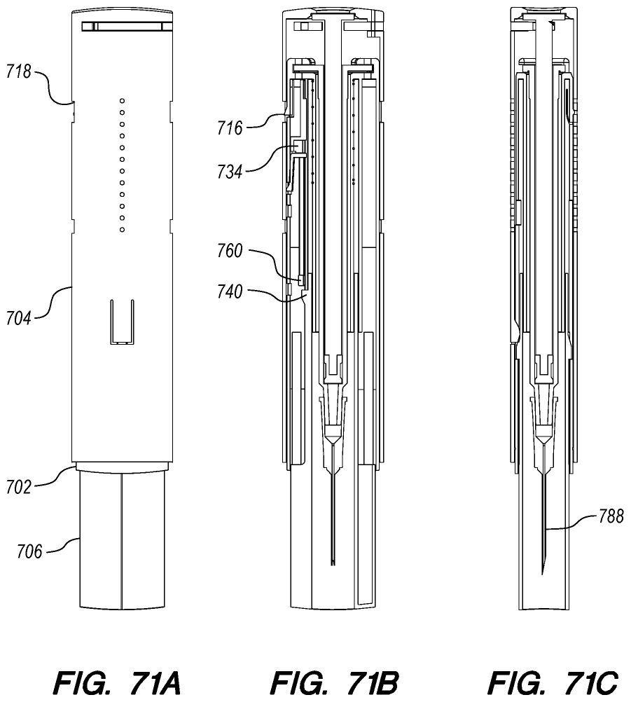

Once the injection has been fully administered into the injection site, the user then stops pushing the device 700 against the site and lifts the device 700 away from the site. At this state, locking tab 716 has now moved from lower hole 732 of top cap 704 to the upper hole 730 of the top cap. The potential energy built up in the compressed spring 766 is also released. Doing so causes the sheath 706 to be pushed downward to surround the needle 788 once again as seen in FIG. 71A. Once at the end of its travel, the sheath 706 is now locked in place via the complete actuation of the sheath lock mechanism 746. As best illustrated in FIG. 71B, movement of the sheath 706 upward and back into the housing 702 is prohibited via sheath stop 760 now disposed above ledge 740 of sheath 706. In this manner, the sheath lock mechanism 746 serves to keep the needle 788 safely and firmly concealed within sheath 706 after use of the device 700.

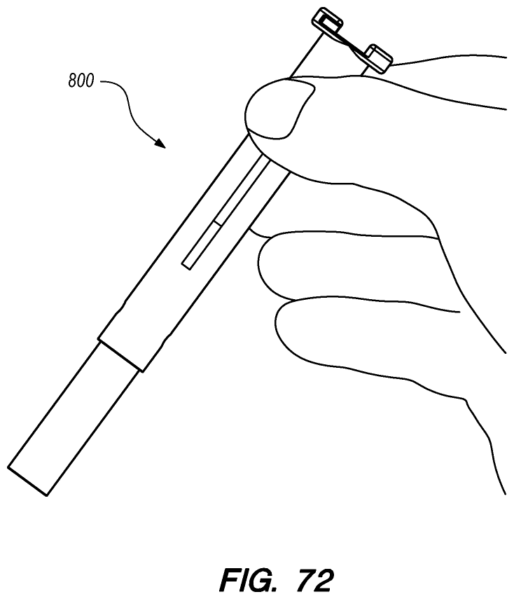

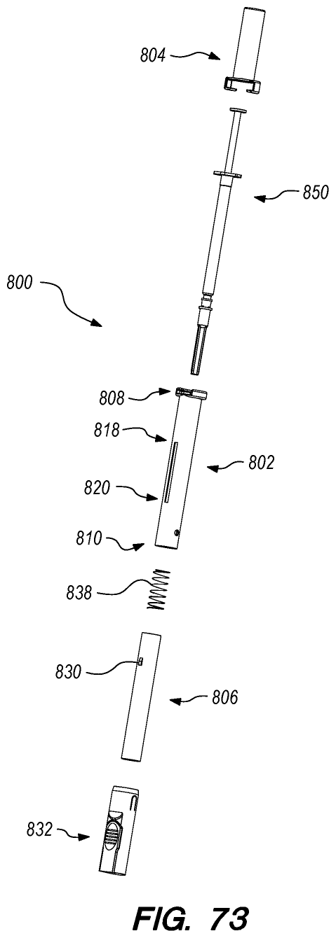

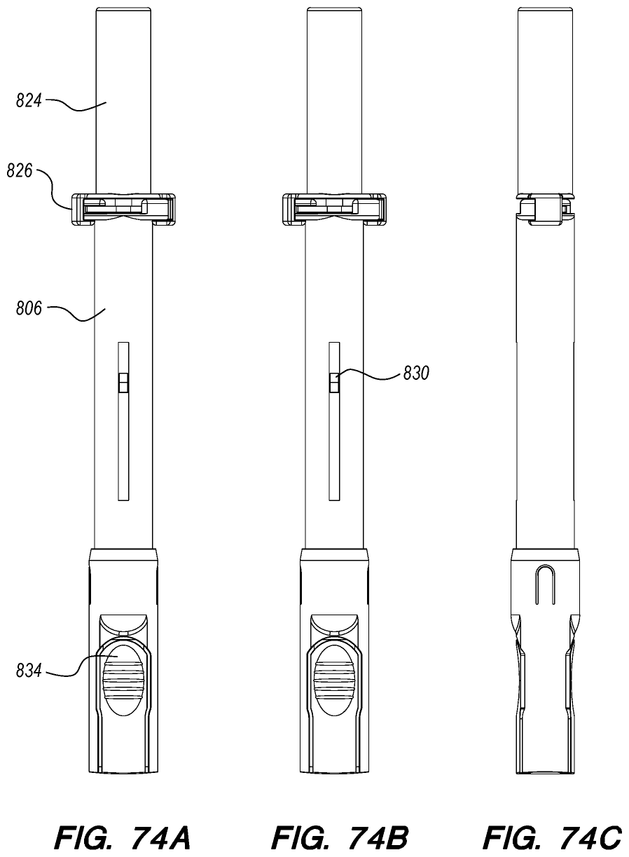

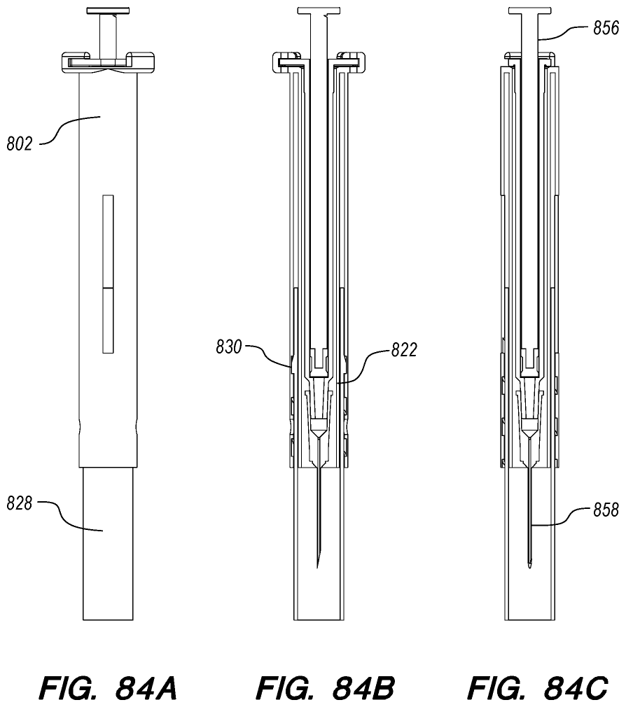

FIGS. 72-84C illustrate another embodiment of a medicament device 800 for administering a shot from a standard pre-filled syringe 850 such as 1 ml Becton Dickinson tuberculin syringe filled with desired medication 860 such as a vaccine for COVID-19. Device 800 may include aspects of the above-disclosed inventions except where noted.

Syringe 850 may comprise barrel 852, plunger 86 that slides within barrel 852, needle 858, and its needle cover 862. The barrel 852 has flanges 854 at one end. The plunger 856 moves within barrel 852 to dispense medication 860. Depending on the intended use of device 800, medication 860 may be injected into a site (not shown) such as a deltoid, triceps, quadriceps or the like of a user or patient.

Medicament device 800 comprises a housing 802 having a top end 808 and an opposing bottom end 810, a top cap 804 disposed about the top end 808, and a sheath 806 disposed about the bottom end 810. Housing 802 includes a syringe catch 814 at the top end 808 and retaining slots 812 cut within the syringe catch 814. The retaining slots 812 retain the flanges 854 of syringe 850. A track 816, which has a top end 818 and an opposing bottom end 820, runs along the body of housing 802. A channel 822 is cut within the interior of housing 802.

The top cap 804 includes a body 824 and arms 826 extending from the body 824 at one end thereof.

The sheath 806 comprises a body 828 with a traveler 830 extending outward therefrom. The track 816 of housing 802 is configured to receive traveler 830 once sheath 806 has been inserted within housing 802. The channel 822 within housing 802 is also configured to receive traveler 830 once a shot has been administered to lock the sheath 806 in place once, as explained further below.

The device 800 preferably includes a spring 838 disposed between the housing 802 and sheath 806.

A sheath cap 838 is disposed about sheath 806. The sheath cap 838 includes finger pads 834 cut therewithin and grippers 836 in the interior thereof.

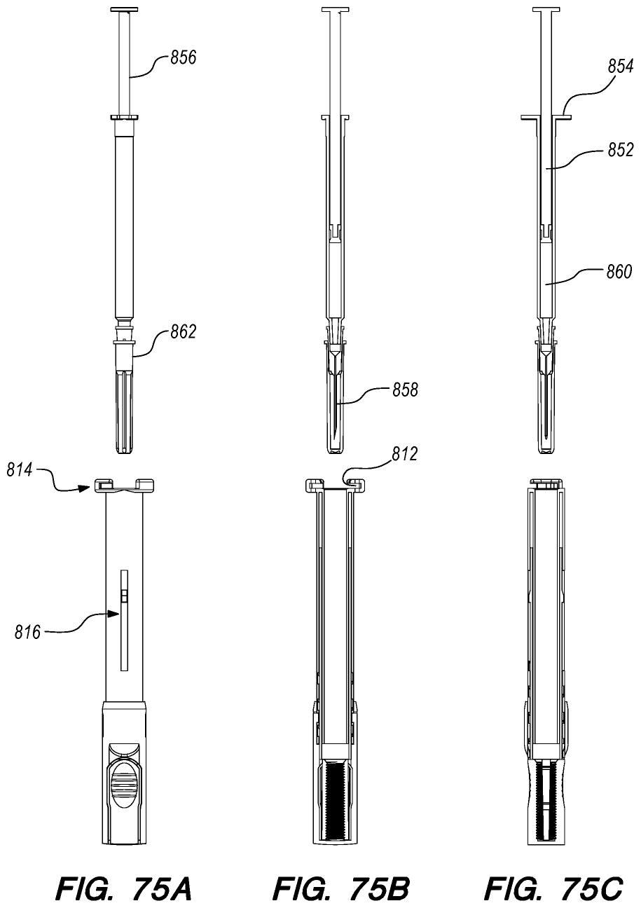

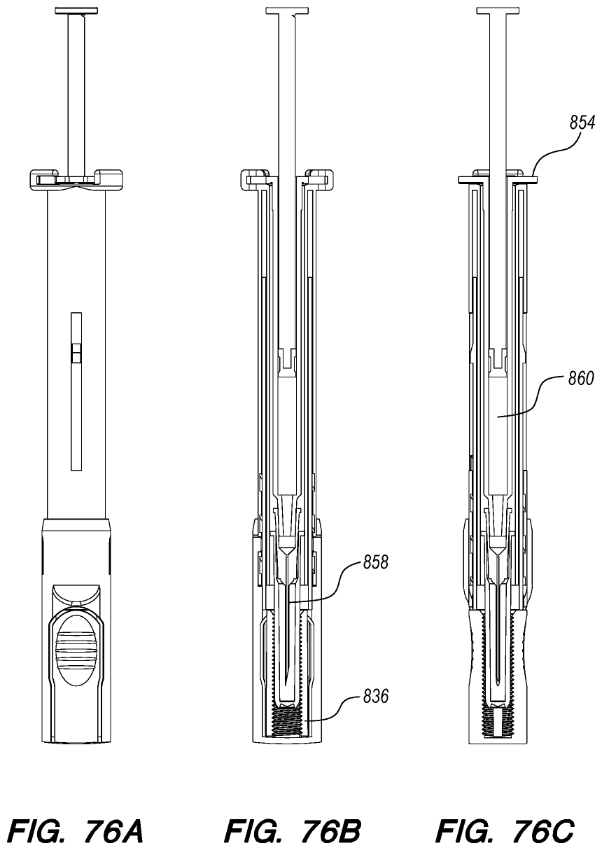

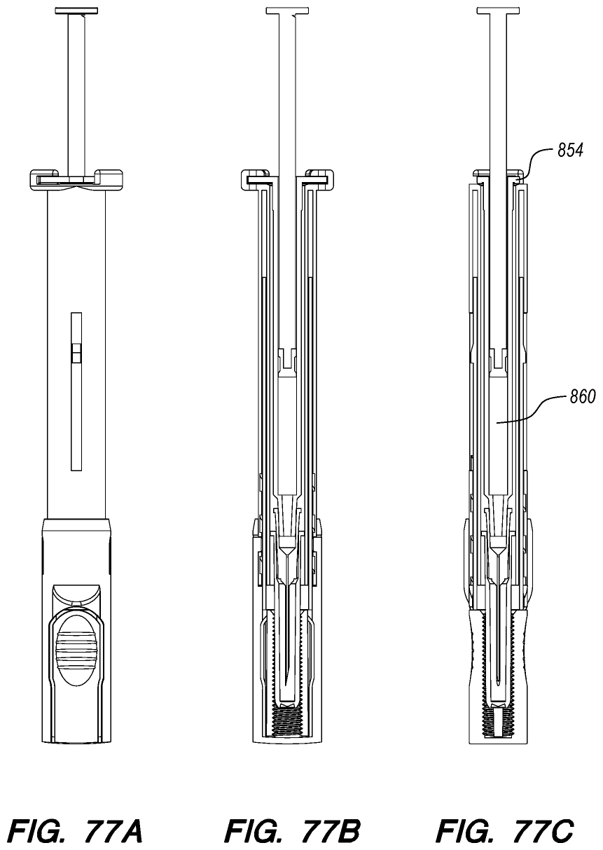

In operation, the user holds device 800 in their hand as seen in FIG. 72. The device 800 in its fully assembled state may come already loaded with syringe 850 and applicable medication 860. Should the device 800 not come with a pre-loaded syringe 80, the user may pull off top cap 804 to ready the device for a syringe 850 with medication 860 to be inserted within housing 802, as illustrated in FIG. 75A. Next, the user drops the filled syringe 850 into the housing 802, with the flanges 854 bottoming out against the top end 808 as seen in FIG. 76A. The user then twists the syringe 850 wherein its flanges 854 seat with retaining slots 812 to keep the syringe 850 in place as seen in FIG. 77A.

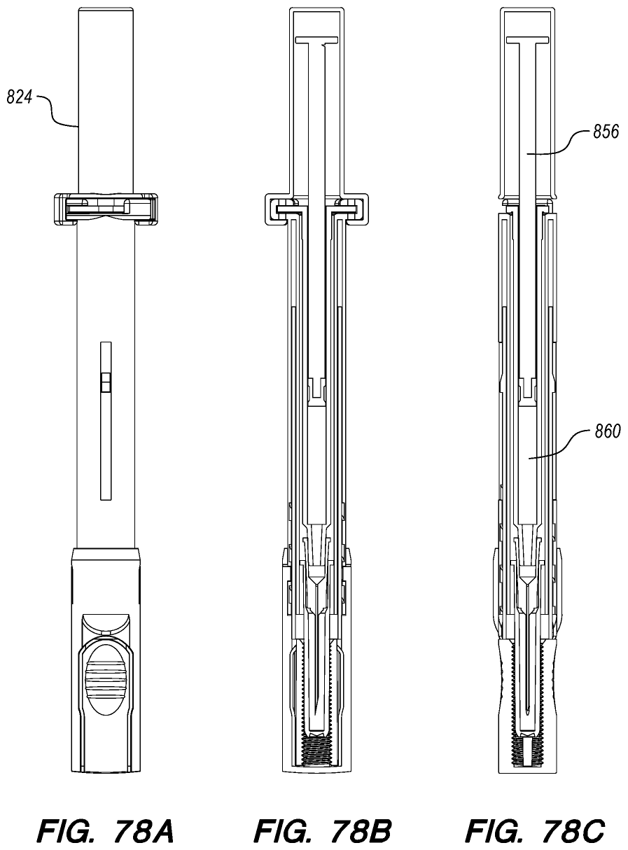

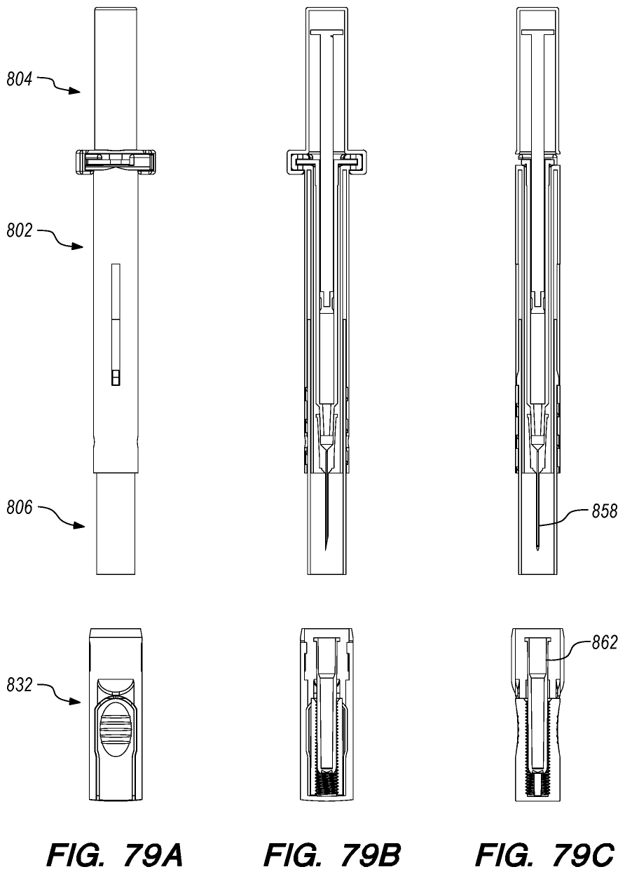

Next, the user then places the top cap 804 onto housing 802 and rotates the body 824 of the top cap until the arms 826 engage the syringe catch 814 as seen in FIG. 78A. The user then pulls downward on the sheath cap 632 and preferably does so by grabbing the finger pads 834 cut within the sheath cap 832. Doing so causes grippers 836 in the interior of sheath cap 832 to grab onto the cover 862 of syringe 850. Further pulling removes the sheath cap 832 from the device 800 wherein cover 862 is now fully contained within sheath cap 832 as illustrated in FIG. 79A.

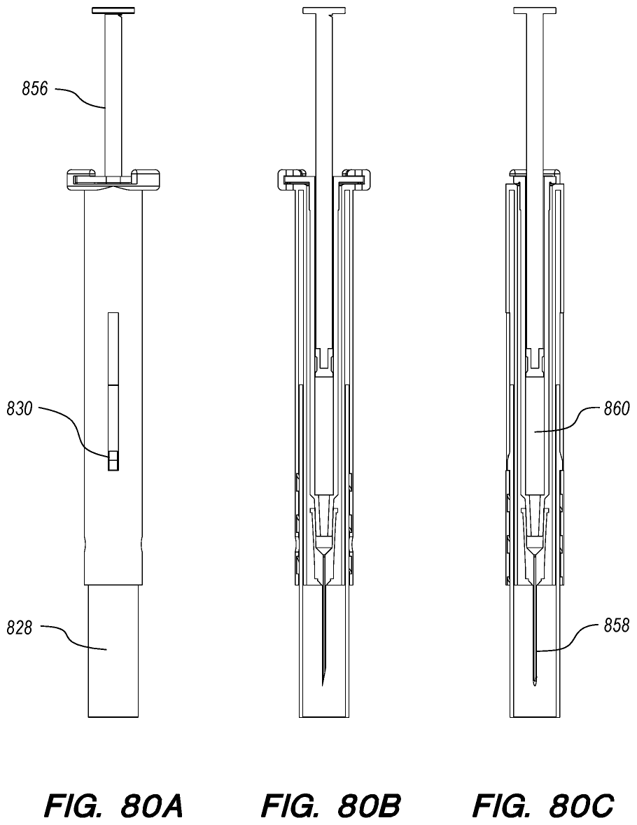

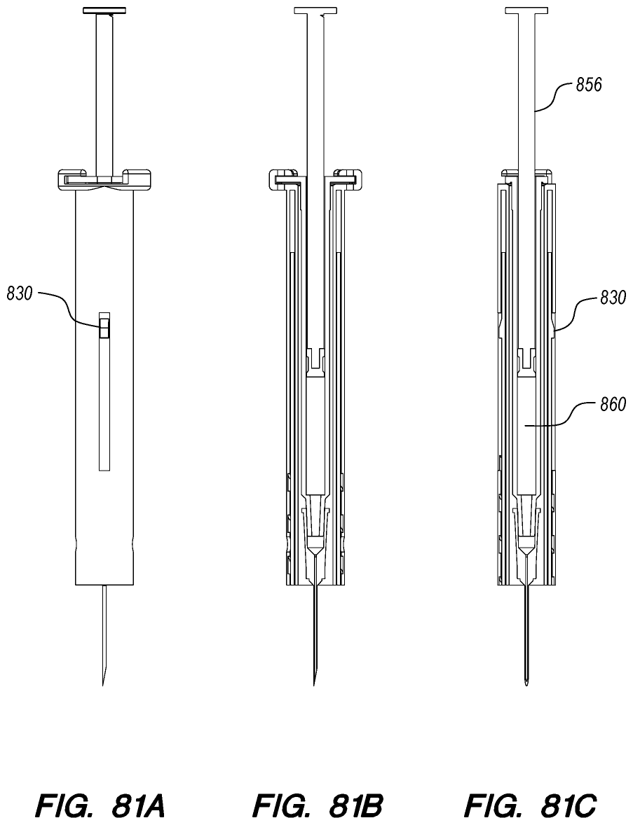

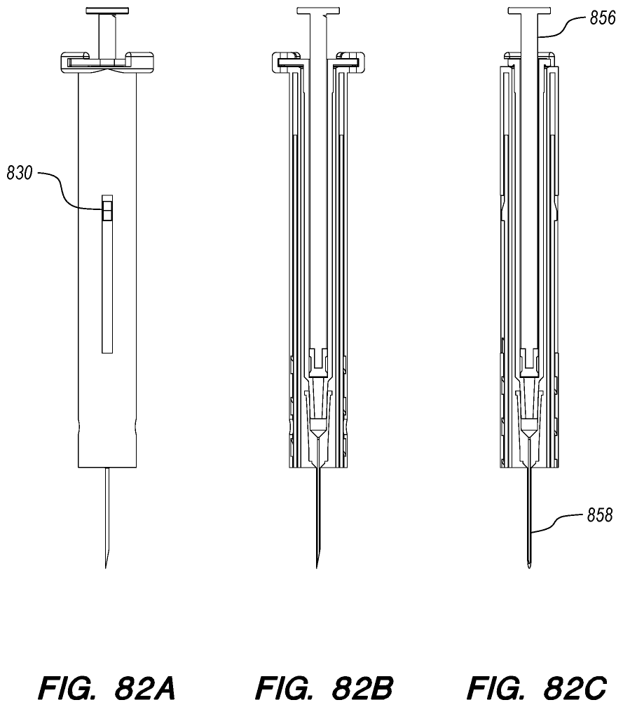

The user then removes top cap 804 by twisting the body 804 until the arms 826 become disengaged from the syringe catch 814 as seen in FIG. 80A. Next, the user begins to administer an injection against an injection site. To do so, sheath 806 is pushed against the injection site, forcing the sheath 806 to move upward into housing 802 as seen in FIG. 81A. At this state, traveler 830 moves upward along track 816 from the track's bottom end 820 toward the track's opposing top end 820, while spring 838 is being compressed. The user then depresses the plunger 856 causing medication 860 to be injected as seen in FIG. 82A.

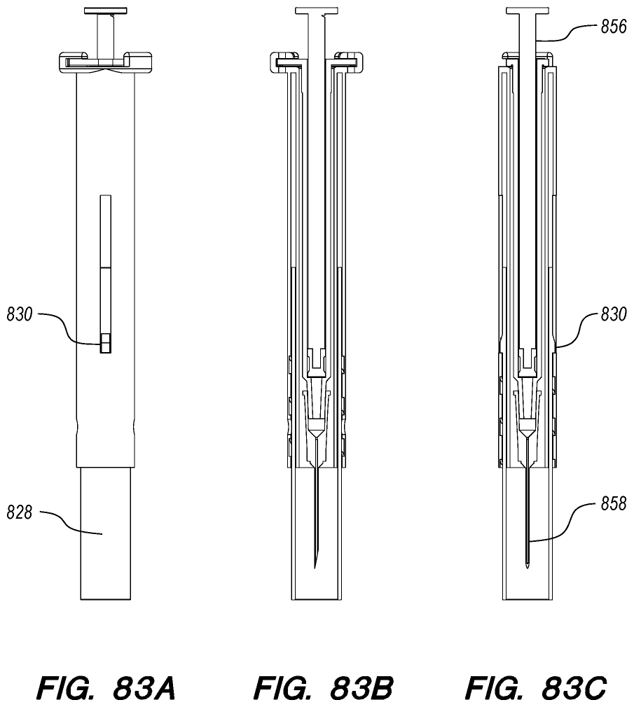

Once the injection has been fully administered into the injection site, the user then stops pushing the device 800 against the site and lifts the device 800 away from the site. The potential energy built up in the compressed spring 838 is released. Doing so causes the sheath 806 to be pushed downward to surround the needle 858 once again as seen in FIG. 83A. Once at the end of its travel, the sheath 806 may now be locked in place. At this point, traveler 830 has moved from the top end 818 of track 816 to bottom end 820 of track 816 (compare position of traveler 830 in FIG. 82A to its position in FIG. 83A). To lock sheath 806 in place, the user rotates the body 828 of sheath 806 to force traveler 830 to move into channel 822 cut within housing 802. Further rotation of the body 828 causes traveler 830 to rotate downward in channel 822, with the user continuing to rotate until the sheath 806 is no longer permitted to move. In this manner, the sheath 806 serves to keep the needle 858 safely and firmly concealed within sheath 806 after use of the device 800.

It should now be apparent that the disclosed inventions have many advantages over conventional devices such as those discussed previously. For example, the disclosed inventions 100, 200, 300, 400, 500, 600, 700, 800 facilitate hypodermic injection using an evolved, standard ubiquitous syringe rather than a proprietary or custom syringe that may fail. Moreover, the disclosed inventions may be filled to a prescribed dose of medicament rather than “one-dose-fits-all” as found with auto-injectors and factory-filled syringes. Such pre-loading of medicament may conveniently be done in a domestic setting such as the user's home, in a clinical setting such as a doctor's office, or in an institutional setting such as a school. Indeed, as the contained syringe is pre-filled to a prescribed dosage, the disclosed inventions are ready for use and may be rapidly deployed and used in emergency or military situations.

The sheath cap of the disclosed inventions also acts as both protector and safety cover against accidental activation.

The disclosed inventions are simpler and safer than administering shots with a standard syringe aspirating from a standard vial. Fortunately, the use of the disclosed inventions is not dissimilar to the customary administering of a typical shot. Such action is in contrast to the violent firing of a needle into skin as is done with a conventional auto-injector. Moreover, because the contained syringe is pre-filled to a prescribed dosage, an unskilled user may quickly and safely administer a shot.

The disclosed inventions may also function with any prescribed quantity of medicament equal to or less than full capacity. Finally, the disclosed inventions may have only two actions to administer a shot: removing the cap, and pushing down the device on an intended injection site. Conventional auto-injectors require removal from a carrier tube, removal of a locking key, and then pushing onto the intended injection site.

While certain embodiments have been described, the embodiments have been presented by way of example only and are not intended to limit the scope of the inventions. Indeed, the medicament devices disclosed herein may be embodied in a variety of other forms. Furthermore, various omissions, substitutions, and changes in the form of the disclosed elements may be made without departing from the spirit of the inventions.

1. A device for administering medicament using a conventional syringe, the device comprising:
a body assembly comprising an upper housing, an inner housing adapted to matingly engage the upper housing, a lower housing receiving the inner housing, and a sheath slideably contained within the lower housing;
an inner cap disposed about the upper housing; and
a sheath lock mechanism comprising a sheath lock disposed within the lower housing, guide rods disposed about the sheath, and springs encircling the guide rods, wherein the sheath lock includes sheath lock legs adapted to engage the sheath to prohibit movement of the sheath after use of the device.

2. The medicament device of claim 1, wherein the body assembly is adapted to receive a conventional syringe, and wherein the device further comprises a plunger lock mechanism for maintaining the syringe inside the body assembly.

3. The medicament device of claim 2, wherein the plunger lock mechanism comprises bayonet lock arms and a bayonet lock channel, wherein the bayonet lock arms extend from the upper housing, and wherein the bayonet lock channel is cut into the inner housing, the bayonet lock channel adapted to receive the bayonet lock arms.

4. The medicament device of claim 3, wherein the sheath is adapted to engage the inner housing once the syringe has been fully inserted into an injection site to disengage the upper housing and inner cap to permit medication inside the syringe to be dispensed.

5. The medicament device of claim 1, further comprising a sheath cap disposed about the lower housing, wherein the sheath cap comprises a needle cover stripper and a stripper cap for receiving the needle cover stripper.

6. The medicament device of claim 5, wherein the needle cover stripper includes ramps on the interior surface thereof, wherein the stripper cap includes cap fingers adapted to engage the ramps.

7. The medicament device of claim 1, wherein the inner housing includes guide rod tunnels, each of which respectively receives one end of one of the guide rods.

8. The medicament device of claim 1, further comprising an outer cap adapted to receive the inner cap, and a wipe pad disposed about the stripper cap.

9. A device for administering medicament using a conventional syringe, the device comprising:
a housing having a top end and an opposing bottom end, wherein the housing includes a track cut into the housing;
a sheath having a body and legs extending upward therefrom, wherein the sheath is configured to slide within the housing; and
a sheath lock mechanism including a sheath lock and a spring, wherein the sheath lock is configured to slide within the track cut into the housing, wherein the spring is disposed between the sheath and the housing,
wherein the sheath lock has a body, a first leg extending downward from the body, a second leg extending downward from the body, and a third leg extending upward from the body,
wherein one of the legs of the sheath has a ledge, wherein the ledge is adapted to engage the sheath lock to prohibit movement of the sheath once medicament within a syringe has been fully administered by a user.

10. The medicament device of claim 9, wherein the first and second legs of the sheath lock are substantially parallel to one another, and wherein the second leg of the sheath lock includes a sheath stop.

11. The medicament device of claim 10, wherein the sheath stop is disposed near the ledge of the sheath during engagement of the sheath lock to prohibit movement of the sheath after use of the device.

12. The medicament device of claim 9, further comprising a top cap configured to slide over the housing, wherein the housing includes a stop hole for receiving a sprung tab extending from the top cap when the top cap has been slid over the housing.

13. The medicament device of claim 12, wherein each leg of the sheath includes a ramp, wherein the ramp is configured to deflect the sprung tab away from the housing to cause the sprung tab to disengage from the stop hole during use of the device.

14. The medicament device of claim 12, further comprising a plunger lock mechanism including a lock slot cut within the top cap, and a plunger lock adapted to fit within the lock slot.

15. The medicament device of claim 9, wherein the housing has a lower hole cut therewithin, and an upper hole cut therewithin, wherein the first leg of the sheath lock has a catch, wherein the catch is adapted to be received in the lower hole during an initial stage of use of the device, and wherein the catch is adapted to be received in the upper hole during a later stage of use of the device by way of the sheath lock sliding upward within the track of the housing.

16. The medicament device of claim 9, further comprising a sheath cap disposed about the sheath before use of the device, wherein the sheath cap includes finger pads cut therewithin, wherein the sheath cap includes grippers in the interior thereof, wherein the grippers are adapted to engage a cover of a syringe to remove the cover during use of the device.

17. A device for administering medicament using a conventional syringe, the device comprising:
a housing having a top end and an opposing bottom end, wherein the housing includes a track cut into a side of the housing running between the top end and bottom end thereof, a syringe catch about the top end of the housing, retaining slots cut within the syringe catch, and a channel cut into an interior of the housing;
a sheath having a body and a traveler extending outward therefrom, wherein the sheath is configured to slide within the housing, wherein the track is adapted to receive the traveler, and wherein the channel is adapted to receive the traveler after medication has been injected by rotating the body of the sheath downward away from the housing; and
a spring disposed between the sheath and the housing.

18. The medicament device of claim 17, further comprising a top cap configured to slide over the housing, wherein the housing includes a body and arms extending away from the body at one end thereof.

19. The medicament device of claim 18, wherein the arms of the top cap are adapted to engage the syringe catch.

20. The medicament device of claim 17, further comprising a sheath cap disposed about the sheath before use of the device, wherein the sheath cap includes finger pads cut therewithin, wherein the sheath cap includes grippers in the interior thereof, wherein the grippers are adapted to engage a cover of a syringe.
